(19) United States
(12) Patent Application Publication
(10) Pub. No.: US 2021/0161997 A1
(43) Pub. Date: Jun. 3, 2021

(54) TREATING DISEASES VIA TARGETED MODULATION OF GENE SIGNALING NETWORKS

(71) Applicant: Camp4 Therapeutics Corporation, Cambridge, MA (US)

(72) Inventors: David A. Bumcrot, Belmont, MA (US); Alfica Sehgal, Cambridge, MA (US); Alla Sigova, Newton, MA (US); Vaishnavi Rajagopal, Andover, MA (US); Brian Schwartz, Somerville, MA (US); Cynthia Smith, Boston, MA (US)

(21) Appl. No.: 17/045,473

(22) PCT Filed: Apr. 8, 2019
(86) PCT No.: PCT/US2019/026402

§ 371 Date: Oct. 5, 2020

Related U.S. Application Data
(60) Provisional application No. 62/653,760, filed on Apr. 6, 2018.

(51) Int. Cl. A61K 38/12; A61K 31/4545; A61K 31/506; A61K 31/497; A61K 31/4422; A61K 31/395; A61K 33/24; A61K 31/422; A61K 31/407; A61K 31/573; A61K 31/53; A61K 31/519; A61K 31/4709; A61K 31/529; A61K 31/4439; A61K 31/4706; A61K 31/517; A61K 31/593; A61K 31/427; A61K 31/4178; A61K 31/5377; A61K 31/196; A61K 31/404; A61K 31/444; A61K 31/5386; A61K 31/18; A61K 31/5025; A61P 1/16

(52) U.S. Cl. CPC A61K 38/12; A61P 1/16; A61K 31/506; A61K 31/497; A61K 31/4422; A61K 31/395; A61K 33/24; A61K 31/422; A61K 31/407; A61K 31/573; A61K 31/53; A61K 31/519; A61K 31/4709; A61K 31/529; A61K 31/4439; A61K 31/4706; A61K 31/517; A61K 31/593; A61K 31/427; A61K 31/4178; A61K 31/5377; A61K 31/196; A61K 31/404; A61K 31/444; A61K 31/5386; A61K 31/18; A61K 31/5025; A61K 31/4545

(57) ABSTRACT

The present invention provides methods and compositions for the treating a patient with a genetic disease, such as fibronectin glomerulopathy, hereditary coproporphyria and others. Methods and compositions are also provided for modulating the disease-associated gene(s) by altering gene signaling networks.

### CROSS REFERENCE TO RELATED APPLICATION

The present application claims priority to, and the benefits of U.S. Provisional Patent Application Ser. No. 62/653,760, filed Apr. 6, 2018, the contents of which are incorporated herein by reference in their entirety.

### FIELD OF THE INVENTION

The present disclosure provides compositions and methods for the treatment of genetic diseases with unmet needs, such as fibronectin glomerulopathy, hereditary coproporphyria and others, in humans.

### BACKGROUND

Inherited genetic diseases can be fatal or result in conditions that require significant medical intervention. Amongst inherited genetic diseases, rare inherited genetic diseases represent a greater medical challenge. There is presently limited ability to approach therapies for inherited diseases, especially rare inherited diseases. For such diseases, control of cell signaling pathways represents an attractive strategy. By manipulating the signaling pathways controlling the disease gene, expression of the gene may be altered or even fine-tuned to achieve desired therapeutic effect. Even a seemingly slight change in gene expression has been shown to have a significant impact on diseases.

Therefore, the present invention provides novel treatment methods for genetic diseases with unmet needs.

### SUMMARY

The present invention discloses the mapping and identification of gene signaling network(s) associated with a number of disease-associated genes. By perturbing the components of the gene signaling network(s), the inventors have identified novel targets, compounds and/or methods that could be utilized to modulate the expression of such genes. Such methods and compositions may be used to develop various therapies for genetic diseases, such as fibronectin glomerulopathy, hereditary coproporphyria and others.

In some embodiments, the present disclosure provides a method of treating a subject with Fibronectin Glomerulopathy by administering to the subject an effective amount of a compound from Table 3 capable of reducing the expression of a FN1 gene. In some embodiments, the present disclosure provides a method of reducing the expression of a FN1 gene in a cell by introducing into the cell an effective amount of a compound from Table 3 capable of altering one or more signaling molecules associated with the regulatory sequence regions (RSRs) or portion thereof of the FN1 gene. In some embodiments, the compound is selected from the group consisting of smoothened agonist, Crizotinib, BGJ398, AZD2858, and Amlodipine Besylate.

In some embodiments, the present disclosure provides a method of treating a subject with Hereditary coproporphyria by administering to the subject an effective amount of a compound from Table 4 capable of increasing the expression of a CPOX gene. In some embodiments, the present disclosure provides a method of increasing the expression of a CPOX gene in a cell by introducing into the cell an effective amount of a compound from Table 4 capable of altering one or more signaling molecules associated with the regulatory sequence regions (RSRs) or portion thereof of the CPOX gene. In some embodiments, the compound is selected from the group consisting of 17-AAG, Cobalt chloride, SKL2001, FICZ, and prednisone.

In some embodiments, the present disclosure provides a method of treating a subject with SERPINC1 Deficiency by administering to the subject an effective amount of a compound from Table 5, Table 14, Table 15 or Table 16 capable of increasing the expression of a SERPINC1 gene. In some embodiments, the present disclosure provides a method of increasing the expression of a SERPINC1 gene in a cell by introducing into the cell an effective amount of a compound from Table 5, Table 14, Table 15 or Table 16 capable of altering one or more signaling molecules associated with the regulatory sequence regions (RSRs) or portion thereof of the SERPINC1 gene. In some embodiments, the compound is selected from the group consisting of OSI-027, PF04691502, CP-673451, Echinomycin, and Pacritinib (SB1518).

In some embodiments, the present disclosure provides a method of treating a subject with Alagille Syndrome by administering to the subject an effective amount of a compound from Table 6 capable of increasing the expression of a JAG1 gene and/or a NOTCH2 gene. In some embodiments, the present disclosure provides a method of increasing the expression of a JAG1 gene and/or a NOTCH2 gene in a cell by introducing into the cell an effective amount of a compound from Table 6 capable of altering one or more signaling molecules associated with the regulatory sequence regions (RSRs) or portion thereof of the JAG1 gene and/or the NOTCH2 gene. In some embodiments, the compound is selected from the group consisting of Merestinib and Torcetrapib.

In some embodiments, the present disclosure provides a method of treating a subject with Glycogen Storage disease 1b by administering to the subject an effective amount of a compound from Table 7 capable of increasing the expression of a SLC37A4 gene. In some embodiments, the present disclosure provides a method of increasing the expression of a SLC37A4 gene in a cell by introducing into the cell an effective amount of a compound from Table 7 capable of altering one or more signaling molecules associated with the regulatory sequence regions (RSRs) or portion thereof of the SLC37A4 gene. In some embodiments, the compound is selected from the group consisting of Echinomycin, prednisone, CP-673451, and cobalt chloride.

In some embodiments, the present disclosure provides a method of treating a subject with Acute Intermittent porphyria by administering to the subject an effective amount of a compound from Table 8 capable of increasing the expression of a HMBS gene. In some embodiments, the present disclosure provides a method of increasing the expression of a HMBS gene in a cell by introducing into the cell an effective amount of a compound from Table 8 capable of altering one or more signaling molecules associated with the regulatory sequence regions (RSRs) or portion thereof of the HMBS gene. In some embodiments, the compound is sotrastaurin.

In some embodiments, the present disclosure provides a method of treating a subject with LECT2 amyloidosis by administering to the subject an effective amount of a compound from Table 9 capable of reducing the expression of a LECT2 gene. In some embodiments, the present disclosure provides a method of reducing the expression of a LECT2 gene in a cell by introducing into the cell an effective amount of a compound from Table 9 capable of altering one or more signaling molecules associated with the regulatory sequence regions (RSRs) or portion thereof of the LECT2 gene. In some embodiments, the compound is selected from the group consisting of calcitrol, 17-AAG and Ritaonavir.

In some embodiments, the present disclosure provides a method of treating a subject with APOL1-associated glomerular disease by administering to the subject an effective amount of a compound from Table 10 or Table 16 capable of reducing the expression of a APOL1 gene. In some embodiments, the present disclosure provides a method of reducing the expression of a APOL1 gene in a cell by introducing into the cell an effective amount of a compound from Table 10 or Table 16 capable of altering one or more signaling molecules associated with the regulatory sequence regions (RSRs) or portion thereof of the APOL1 gene. In some embodiments, the compound is selected from the group consisting of Nitrofurantoin, Crizotinib, Momelotenib, and Momelotenib metabolite M21.

In some embodiments, the present disclosure provides a method of treating a subject with Gilbert Syndrome or Criggler Najjar, type II by administering to the subject an effective amount of a compound from Table 11 capable of increasing the expression of a UGT1A1 gene. In some embodiments, the present disclosure provides a method of increasing the expression of a UGT1A1 gene in a cell by introducing into the cell an effective amount of a compound from Table 11 capable of altering one or more signaling molecules associated with the regulatory sequence regions (RSRs) or portion thereof of the UGT1A1 gene. In some embodiments, the compound is selected from the group consisting of FICZ, Kartogenin, meBIO, CP-673451, BAM7, and EW-7197.

In some embodiments, the present disclosure provides a method of treating a subject with dyslipidemia by administering to the subject an effective amount of a compound from Table 12 or Table 13 capable of increasing the expression of a LDLR gene, and/or reducing the expression of a ANGPTL3 gene and/or PCSK9 gene. In some embodiments, the present disclosure provides a method of modulating the expression of at least one gene selected from the group consisting of ANGPTL3, LDLR, and PCSK9 genes in a cell by introducing into the cell an effective amount of a compound from Table 12 or Table 13 capable of altering one or more signaling molecules associated with the regulatory sequence regions (RSRs) or portion thereof of the ANGPTL3, LDLR, or PCSK9 genes. In some embodiments, the compound is selected from the group consisting of WYE-125132, Pifithrin-u, LY294002, SGI-1776, Preladenant, and CO-1686.

In some embodiments, the present disclosure provides a method of treating a subject with Rett Syndrome, comprising administering to the subject an effective amount of a compound from Table 15 capable of increasing the expression of a MECP2 gene. In some embodiments, the present disclosure provides a method of treating a subject with Rett Syndrome, comprising administering to the subject an effective amount of a compound from Table 15 capable of increasing the expression of a MECP2 gene. In some embodiments, the compound is 17-AAG.

In certain embodiments, the subject is human.

### BRIEF DESCRIPTION OF THE DRAWINGS

The foregoing and other objects, features and advantages will be apparent from the following description of particular embodiments of the disclosure, as illustrated in the accompanying drawings. The drawings are not necessarily to scale; emphasis instead being placed upon illustrating the principles of various embodiments of the disclosure.

FIG. 1 illustrates the packaging of chromosomes in a nucleus, the localized topological domains into which chromosomes are organized, insulated neighborhoods in TADs and finally an example of an arrangement of a signaling center(s) around a particular disease gene.

FIG. 2A and FIG. 2B illustrate a linear and 3D arrangement of the CTCF boundaries of an insulated neighborhood.

FIG. 3A and FIG. 3B illustrate tandem insulated neighborhoods and gene loops formed in such insulated neighborhoods.

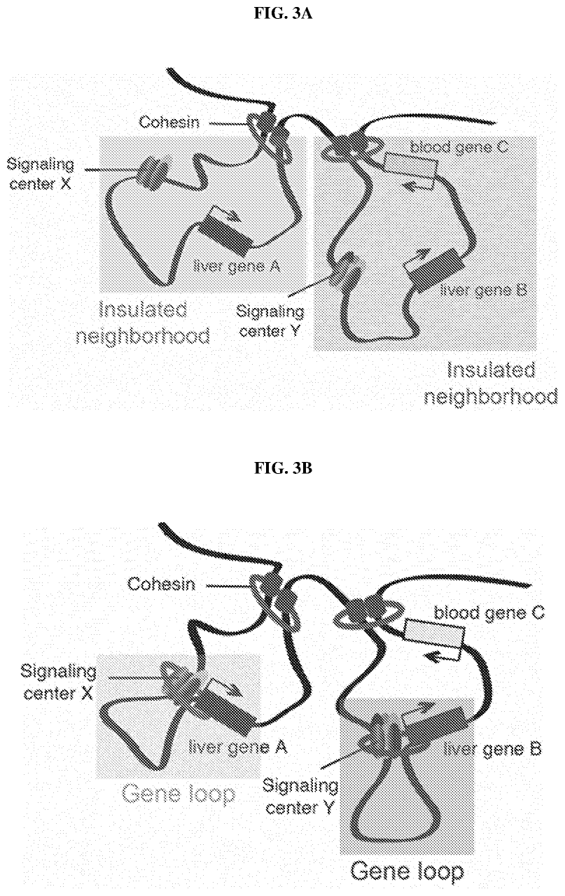

FIG. 4 illustrates the concept of an insulated neighborhood contained within a larger insulated neighborhood and the signaling which may occur in each.

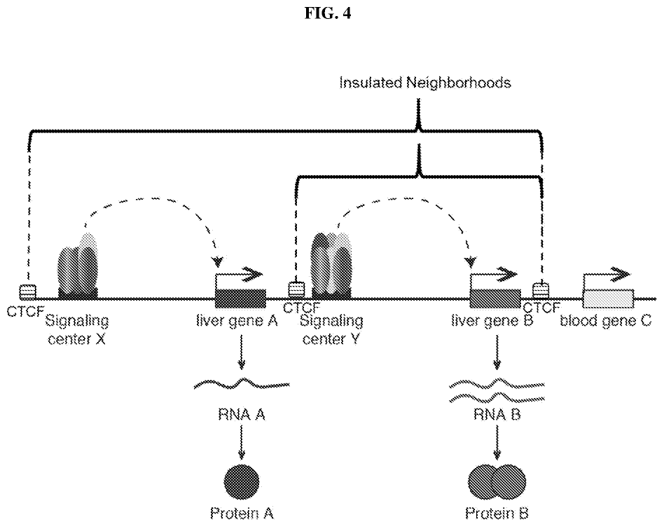

FIG. 5 illustrates the components of a signaling center; including transcriptional factors, signaling proteins, and/or chromatin regulators.

FIG. 6 shows fold change increase in SERPINC1 mRNA relative to PPIA after 72 h mTOR inhibition by siRNA in HU4282 primary human hepatocytes.

FIG. 7 shows dose dependent fold change increase of SERPINC1 mRNA after 72 h treatment with compound 308 (OSI-027) and compound 309 (PF04691502) in HU4282 primary human hepatocytes relative to DMSO control.

FIG. 8 shows fold change increase in MECP2 mRNA in mouse hepatocytes following treatment with 10 uM 17-AAG.

FIG. 9 shows fold change increase in MECP2 mRNA in mouse liver following treatment with 10 uM 17-AAG.

FIG. 10A shows fold change increase in MECP2 mRNA in human hepatocytes following treatment with 17-AAG at the indicated dosages or DMSO from hepatocytes isolated from donor 1.

FIG. 10B shows fold change increase in MECP2 mRNA in human hepatocytes following treatment with 17-AAG at the indicated dosages or DMSO from hepatocytes isolated from donor 2.

FIG. 11 shows the fold change in APOL1 mRNA in primary human hepatocytes following treatment with 3.3 uM Momelotinib or DMSO.

FIG. 12 shows the fold change in APOL1 mRNA in primary human hepatocytes following treatment with 3.3 uM Momelotinib (MMB), M21 Momelotinib metabolite (M21) or DMSO.

### DETAILED DESCRIPTION

### I. INTRODUCTION AND DEFINITIONS

The present disclosure provides compositions and methods for the treatment of genetic diseases, such as fibronectin glomerulopathy, hereditary coproporphyria and others, in humans In particular, the disclosure provides compounds and related use for the modulation of the disease-associated gene(s), such as FN1, CPOX, and others.

The present disclosure also embraces the alteration, perturbation and ultimate regulated control of gene signaling networks (GSNs). Such gene signaling networks include genomic signaling centers found within insulated neighborhoods of the genomes of biological systems. Compounds modulating gene expression may act through modulating one or more gene signaling networks.

As used herein, a “gene signaling network” or “GSN” comprises the set of biomolecules associated with any or all of the signaling events from a particular gene, e.g., a gene-centric network. As there are over 20,000 protein-coding genes in the human genome, there are at least this many gene signaling networks. And to the extent some genes are non-coding genes, the number increases greatly. Gene signaling networks differ from canonical signaling pathways which are mapped as standard protein cascades and feedback loops.

Traditionally, signaling pathways have been identified using standard biochemical techniques and, for the most part, are linear cascades with one protein product signaling the next protein product-driven event in the cascade. While these pathways may bifurcate or have feedback loops, the focus has been almost exclusively at the protein level.

Gene signaling networks (GSNs) of the present disclosure represent a different paradigm to defining biological signaling—taking into account protein-coding and nonprotein-coding signaling molecules, genomic structure, chromosomal occupancy, chromosomal remodeling, the status of the biological system and the range of outcomes associated with the perturbation of any biological systems comprising such gene signaling networks.

Genomic architecture, while not static, plays an important role in defining the framework of the GSNs of the present disclosure. Such architecture includes the concepts of chromosomal organization and modification, topologically associated domains (TADs), insulated neighborhoods (INs), genomic signaling centers (GSCs), signaling molecules and their binding motifs or sites, and of course, the genes encoded within the genomic architecture.

The term “insulated neighborhood” (IN), as used herein, refers to chromosome structure formed by the looping of two interacting sites in the chromosome sequence that may comprise CCCTC-Binding factor (CTCF) co-occupied by cohesin and affect the expression of genes in the insulated neighborhood as well as those genes in the vicinity of the insulated neighborhoods.

The term “genomic signaling center”, i.e., a “signaling center”, as used herein, refers to regions within insulated neighborhoods that include regions capable of binding context-specific combinatorial assemblies of signaling molecules/signaling proteins that participate in the regulation of the genes within that insulated neighborhood or among more than one insulated neighborhood.

The present disclosure, by elucidating a more definitive set of connectivities of the GSNs associated with the disease-associated target gene(s), provides a fine-tuned mechanism to address genetic diseases, such as fibronectin glomerulopathy, hereditary coproporphyria and others.

### Genomic Architecture

Cells control gene expression using thousands of elements that link cellular signaling to the architecture of the genome. Genomic system architecture includes regions of DNA, RNA transcripts, chromatin remodelers, and signaling molecules.

### Chromosomes

Chromosomes are the largest subunit of genome architecture that contain most of the DNA in humans. Specific chromosome structures have been observed to play important roles in gene control, as described in Hnisz et al., Cell 167, Nov. 17, 2016, which is hereby incorporated by reference in its entirety. The introns (“non-coding regions”) provide protein binding sites and other regulatory structures, while the exons encode for signaling molecules, such as transcription factors, that interact with the non-coding regions to regulate gene expression. DNA sites within non-coding regions on the chromosome also interact with each other to form looped structures. These interactions form a chromosome scaffold that is preserved through development and plays an important role in gene activation and repression. Interactions rarely occur among chromosomes and are usually within the same domain of a chromosome.

In situ hybridization techniques and microscopy have revealed that individual interphase chromosomes tend to occupy small portions of the nucleus and do not spread throughout this organelle. See, Cremer and Cremer, Cold Spring Harbor Perspectives in Biology 2, a003889, 2010, which is hereby incorporated by reference in its entirety. This small surface area occupancy might reduce interactions between chromosomes.

### Topologically Associating Domains (TADs)

The term “topologically associating domains” (TADs), as used herein, refers to structures that represent a modular organization of the chromatin and have boundaries that are shared by the different cell types of an organism. Topologically Associating Domains (TADs), alternatively known as topological domains, are hierarchical units that are subunits of the mammalian chromosome structure. See, Dixon et al., Nature, 485(7398):376-80, 2012; Filippova et al., Algorithms for Molecular Biology, 9:14, 2014; Gibcus and Dekker Molecular Cell, 49(5):773-82, 2013; Naumova et al., Science, 42(6161):948-53, 2013; which are hereby incorporated by reference in their entireties. TADs are megabase-sized chromosomal regions that demarcate a microenvironment that allows genes and regulatory elements to make productive DNA-DNA contacts. TADs are defined by DNA-DNA interaction frequencies. The boundaries of TADs consist of regions where relatively fewer DNA-DNA interactions occur, as described in Dixon et al., Nature, 485(7398):376-80, 2012; Nora et al., Nature, 485(7398):381-5, 2012; which are hereby incorporated by reference in their entirety. TADs represent structural chromosomal units that function as gene expression regulators.

TADs may contain about 7 or more protein-coding genes and have boundaries that are shared by the different cell types. See, Smallwood et al., Current Opinion in Cell Biology, 25(3):387-94, 2013, which is hereby incorporated by reference in its entirety. Some TADs contain active genes and others contain repressed genes, as the expression of genes within a single TAD is usually correlated. See, Cavalli et al., Nature Structural & Molecular Biology, 20(3):290-9, 2013, which is hereby incorporated by reference in its entirety. Sequences within a TAD find each other with high frequency and have concerted, TAD-wide histone chromatin signatures, expression levels, DNA replication timing, lamina association, and chromocenter association. See, Dixon et al., Nature, 485(7398):376-80, 2012; Le Dily et al., Genes Development, 28:2151-62, 2014; Dixon et al., Nature, 485(7398):376-80, 2012; Wijchers, Genome Research, 25:958-69, 2015, which are hereby incorporated by reference in their entireties.

Gene loops and other structures within TADs influence the activities of transcription factors (TFs), cohesin, and 11-zinc finger protein (CTCF), a transcriptional repressor. See, Baranello et al., Proceedings of the National Academy of Sciences, 111(3):889-9, 2014, which is hereby incorporated by reference in its entirety. The structures within TADs include cohesin-associated enhancer-promoter loops that are produced when enhancer-bound TFs bind cofactors, for example Mediator, that, in turn, bind RNA polymerase II at promoter sites. See, Lee and Young, Cell, 152(6):1237-51, 2013; Lelli et al., 2012; Roeder, Annual Reviews Genetics 46:43-68, 2005; Spitz and Furlong, Nature Reviews Genetics, 13(9):613-26, 2012; Dowen et al., Cell, 159(2): 374-387, 2014; Lelli et al., Annual Review of Genetics, 46:43-68, 2012, which are hereby incorporated by reference in their entireties. The cohesin-loading factor Nipped-B-like protein (NIPBL) binds Mediator and loads cohesin at these enhancer-promoter loops. See, Kagey et al., Nature, 467(7314):430-5, 2010, which is hereby incorporated by reference in its entirety.

TADs have similar boundaries in all human cell types examined and constrain enhancer-gene interactions. See, Dixon et al., Nature, 518:331-336, 2015; Dixon et al., Nature, 485:376-380, 2012, which are hereby incorporated by reference in their entirety. This architecture of the genome helps explain why most DNA contacts occur within the TADs and enhancer-gene interactions rarely occur between chromosomes. However, TADs provide only partial insight into the molecular mechanisms that influence specific enhancer-gene interactions within TADs.

Long-range genomic contacts segregate TADs into an active and inactive compartment. See, Lieberman-Aiden et al., Science, 326:289-93, 2009, which is hereby incorporated by reference in its entirety. The loops formed between TAD boundaries seem to represent the longest-range contacts that are stably and reproducibly formed between specific pairs of sequences. See, Dixon et al., Nature, 485(7398):376-80, 2012, which is hereby incorporated by reference in its entirety.

In some embodiments, the methods of the present disclosure are used to alter gene expression from genes located in a TAD. In some embodiments, TAD regions are modified to alter gene expression of a non-canonical pathway as defined herein or as definable using the methods described herein.

### Insulated Neighborhoods

As used herein, an “insulated neighborhood” (IN) is defined as a chromosome structure formed by the looping of two interacting sites in the chromosome sequence. These interacting sites may comprise CCCTC-Binding factor (CTCF). These CTCF sites are often co-occupied by cohesin. The integrity of these cohesin-associated chromosome structures affects the expression of genes in the insulated neighborhood as well as those genes in the vicinity of the insulated neighborhoods. A “neighborhood gene” is a gene localized within an insulated neighborhood. Neighborhood genes may be coding or non-coding.

Insulated neighborhood architecture is defined by at least two boundaries which come together, directly or indirectly, to form a DNA loop. The boundaries of any insulated neighborhood comprise a primary upstream boundary and a primary downstream boundary. Such boundaries are the outermost boundaries of any insulated neighborhood. Within any insulated neighborhood loop, however, secondary loops may be formed. Such secondary loops, when present, are defined by secondary upstream boundaries and secondary downstream boundaries, relative to the primary insulated neighborhood. Where a primary insulated neighborhood contains more than one internal loop, the loops are numbered relative to the primary upstream boundary of the primary loop, e.g., the secondary loop (first loop within the primary loop), the tertiary loop (second loop within the primary loop), the quaternary loop (the third loop within the primary loop) and so on.

Insulated neighborhoods may be located within topologically associated domains (TADs) and other gene loops. TADs are defined by DNA-DNA interaction frequencies, and average 0.8 Mb, contain approximately 7 protein-coding genes and have boundaries that are shared by the different cell types of an organism. According to Dowen, the expression of genes within a TAD is somewhat correlated, and thus some TADs tend to have active genes and others tend to have repressed genes. Dowen et al., Cell. 2014 Oct. 9; 159(2): 374-387.

Insulated neighborhoods may exist as contiguous entities along a chromosome or may be separated by non-insulated neighborhood sequence regions. Insulated neighborhoods may overlap linearly only to be defined once the DNA looping regions have been joined. While insulated neighborhoods may comprise 3-12 genes, they may contain, 1, 2, 3, 4, 5, 6, 7, 8, 9, 10, 11, 12, 13 or more genes.

A “minimal insulated neighborhood” is an insulated neighborhood having at least one neighborhood gene and associated regulatory sequence region or regions (RSRs) which facilitate the expression or repression of the neighborhood gene such as a promoter and/or enhancer and/or repressor region, and the like. It is contemplated that regulatory sequence regions may coincide or even overlap with an insulated neighborhood boundary. Regulatory sequence regions, as used herein, include but are not limited to regions, sections, sites or zones along a chromosome whereby interactions with signaling molecules occur in order to alter expression of a neighborhood gene. As used herein, a “signaling molecule” is any entity, whether protein, nucleic acid (DNA or RNA), organic small molecule, lipid, sugar or other biomolecule, which interacts directly, or indirectly, with a regulatory sequence region on a chromosome. Regulatory sequence regions (RSRs) may also be referred to as “genomic signaling centers” or “GSCs.”

One category of specialized signaling molecules are transcription factors. “Transcription factors” are those signaling molecules which alter, whether to increase or decrease, the transcription of a target gene, e.g., a neighborhood gene.

According to the present disclosure, neighborhood genes may have any number of upstream or downstream genes along the chromosome. Within any insulated neighborhood, there may be one or more, e.g., one, two, three, four or more, upstream and/or downstream neighborhood genes relative to the primary neighborhood gene. A “primary neighborhood gene” is a gene which is most commonly found within a specific insulated neighborhood along a chromosome. An upstream neighborhood gene of a primary neighborhood gene may be located within the same insulated neighborhood as the primary neighborhood gene. A downstream neighborhood gene of a primary neighborhood gene may be located within the same insulated neighborhood as the primary neighborhood gene.

The present disclosure provides methods of altering the penetrance of a gene or gene variant. As used herein, “penetrance” is the proportion of individuals carrying a particular variant of a gene (e.g., mutation, allele or generally a genotype, whether wild type or not) that also exhibits an associated trait (phenotype) of that variant gene. In some situations of disease, penetrance of a disease-causing mutation measured as the proportion of individuals with the mutation who exhibit clinical symptoms. Consequently, penetrance of any gene or gene variant exists on a continuum.

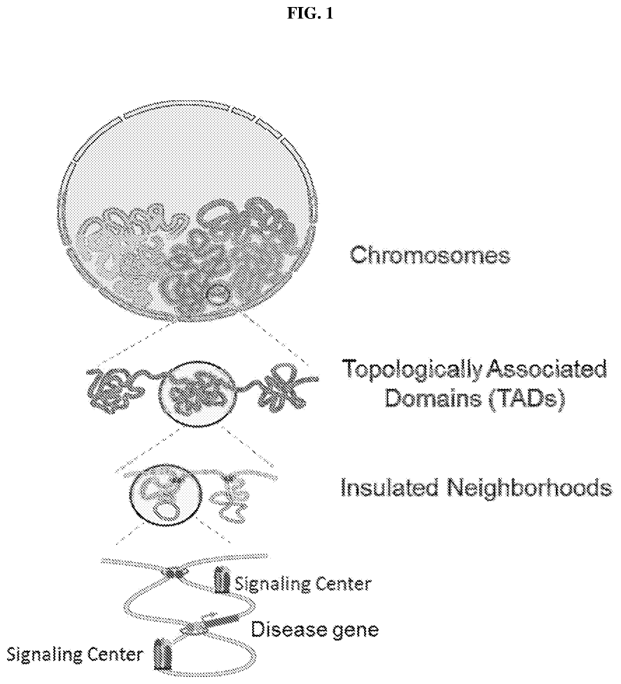

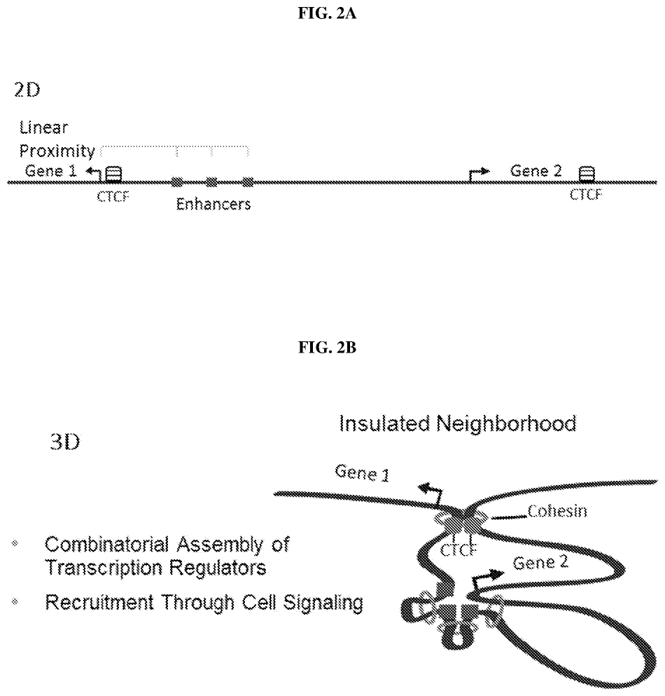

Insulated neighborhoods are functional units that group genes under the same control mechanism, which are described in Dowen et al., Cell, 159: 374-387 (2014), which is hereby incorporated by reference in its entirety. Insulated neighborhoods provide the mechanistic background for higher-order chromosome structures, such as TADs which are shown in FIG. 1. Insulated neighborhoods are chromosome structures formed by the looping of the two interacting CTCF sites co-occupied by cohesin as shown in FIG. 1. The integrity of these structures is important for proper expression of local genes. Generally, 1 to 10 genes are clustered in each neighborhood with a median number of 3 genes within each one. The genes controlled by the same insulated neighborhood are not readily apparent from a two-dimensional view of DNA. In humans, there are about 13,801 insulated neighborhoods in a size range of 25 kb-940 kb with a median size of 186 kb. Insulated neighborhoods are conserved among different cell types. Smaller INs that occur within a bigger IN are referred to as nested insulated neighborhoods (NINs). TADs can consist of a single IN, or one IN and one NIN and two NINs as shown in FIG. 2B.

As used herein, the term “boundary” refers to a point, limit, or range indicating where a feature, element, or property ends or begins. Accordingly, an “insulated neighborhood boundary” refers to a boundary that delimits an insulated neighborhood on a chromosome. According to the present disclosure, an insulated neighborhood is defined by at least two insulated neighborhood boundaries, a primary upstream boundary and a primary downstream boundary. The “primary upstream boundary” refers to the insulated neighborhood boundary located upstream of a primary neighborhood gene. The “primary downstream boundary” refers to the insulated neighborhood boundary located downstream of a primary neighborhood gene. Similarly, when secondary loops are present as shown in FIG. 2B, they are defined by secondary upstream and downstream boundaries. A “secondary upstream boundary” is the upstream boundary of a secondary loop within a primary insulated neighborhood, and a “secondary downstream boundary” is the downstream boundary of a secondary loop within a primary insulated neighborhood. The directionality of the secondary boundaries follows that of the primary insulated neighborhood boundaries.

Components of an insulated neighborhood boundary may comprise the DNA sequences at the anchor regions and associated factors (e.g., CTCF, cohesin) that facilitate the looping of the two boundaries. The DNA sequences at the anchor regions may contain at least one CTCF binding site. Experiments using the ChIP-exo technique revealed a 52 bp CTCF binding motif containing four CTCF binding modules (see FIG. 1, Ong and Corces, Nature reviews Genetics, 12:283-293, 2011, which is incorporated herein by reference in its entirety). The DNA sequences at the insulated neighborhood boundaries may contain insulators. In some cases, insulated neighborhood boundaries may also coincide or overlap with regulatory sequence regions, such as enhancer-promoter interaction sites.

In some embodiments of the present disclosure, disrupting or altering an insulated neighborhood boundary may be accomplished by altering specific DNA sequences (e.g., CTCF binding sites) at the boundaries. For example, existing CTCF binding sites at insulated neighborhood boundaries may be deleted, mutated, or inverted. Alternatively, new CTCF binding sites may be introduced to form new insulated neighborhoods. In other embodiments, disrupting or altering an insulated neighborhood boundary may be accomplished by altering the histone modification (e.g., methylation, demethylation) at the boundaries. In other embodiments, disrupting or altering an insulated neighborhood boundary may be accomplished by altering (e.g., blocking) the binding of CTCF and/or cohesin to the boundaries. In cases where insulated neighborhood boundaries coincide or overlap with regulatory sequence regions, disrupting or altering an insulated neighborhood boundary may be accomplished by altering the regulatory sequence regions (RSR) or the binding of the RSR-associated signaling molecules.

Controlling Expression from Insulated Neighborhoods: Signaling Centers

Historically, the term “signaling center” has been used to describe a group of cells responding to changes in the cellular environment. See, Guger et al., Developmental Biology 172: 115-125 (1995), which is incorporated by reference herein in its entirety. Similarly, the term “signaling center”, as used herein, refers to a defined region of a living organism that interacts with a defined set of biomolecules, such as signaling proteins or signaling molecules (e.g., transcription factors) to regulate gene expression in a context-specific manner

Signaling centers have been discovered to regulate the activity of insulated neighborhoods. These regions control which genes are expressed and the level of expression in the human genome. Loss of the structural integrity of signaling centers contributes to deregulation of gene expression and potentially causing disease.

Signaling centers include enhancers bound by a highly context-specific combinatorial assemblies of transcription factors. These factors are recruited to the site through cellular signaling. Signaling centers include multiple genes that interact to form a three-dimensional transcription factor hub macrocomplex. Signaling centers are generally associated with one to four genes in a loop organized by biological function.

The compositions of each signaling center has a unique composition including the assemblies of transcription factors, the transcription apparatus, and chromatin regulators. Signaling centers are highly context specific, permitting drugs to control response by targeting signaling pathways.

Multiple signaling centers may interact to control the different combinations of genes within the same insulated neighborhood.

### Binding Sites for Signaling Molecules

A series of consensus binding sites, or binding motifs for binding sites, for signaling molecules has been identified by the present inventors. These consensus sequences reflect binding sites along a chromosome, gene, or polynucleotide for signaling molecules or for complexes which include one or more signaling molecules.

In some embodiments, binding sites are associated with more than one signaling molecule or complex of molecules.

### Enhancers

The term “enhancer”, as used herein, refers to regulatory DNA sequences that, when bound by transcription factors, enhance the transcription of an associated gene Enhancers are gene regulatory elements that control cell type specific gene expression programs in humans. See, Buecker and Wysocka, Trends in genetics: TIG 28, 276-284, 2012; Heinz et al., Nature reviews Molecular Cell Biology, 16:144-154, 2015; Levine et al., Cell, 157:13-25, 2014; Ong and Corces, Nature reviews Genetics, 12:283-293, 2011; Ren and Yue, Cold Spring Harbor symposia on quantitative biology, 80:17-26, 2015, which are hereby incorporated by reference in their entireties. Enhancers are segments of DNA that are generally a few hundred base pairs in length and are occupied by multiple transcription factors that recruit co-activators and RNA polymerase II to target genes. See, Bulger and Groudine, Cell, 144:327-339, 2011; Spitz and Furlong, Nature reviews Genetics, 13:613-626, 2012; Tjian and Maniatis, Cell, 77:5-8, 1994, which are hereby incorporated by reference in their entireties Enhancer RNA molecules transcribed from these regions of DNA also “trap” transcription factors capable of binding DNA and RNA. A region with more than one enhancer is a “super-enhancer.” The term “super-enhancers”, as used herein, refers to clusters of transcriptional enhancers that drive expression of genes that define cell identity.

Insulated neighborhoods provide a microenvironment for specific enhancer-gene interactions that are vital for both normal gene activation and repression. Transcriptional enhancers control over 20,000 protein-coding genes to maintain cell type-specific gene expression programs in all human cells. Tens of thousands of enhancers are estimated to be active in any given human cell type. See, ENCODE Project Consortium et al., Nature, 489, 57-74, 2012; Roadmap Epigenomics et al., Nature, 518, 317-330, 2015, which are hereby incorporated by reference in their entirety Enhancers and their associated factors can regulate expression of genes located upstream or downstream by looping to the promoters of these genes. Cohesin ChIA-PET studies carried out to gain insight into the relationship between transcriptional control of cell identity and control of chromosome structure reveal that majority of the super-enhancers and their associated genes occur within large loops that are connected through interacting CTCF-sites co-occupied by cohesin. Such super-enhancer domains (SD) usually contain one super-enhancer that loops to one gene within the SD and the SDs appear to restrict super-enhancer activity to genes within the SD. The correct association of super-enhancers and their target genes in insulated neighborhoods is highly vital because the mis-targeting of a single super-enhancer is sufficient to cause disease. See Groschel et al., Cell, 157(2):369-81, 2014.

Most of the disease-associated non-coding variation occurs in the vicinity of enhancers and hence might impact these enhancer target genes. Therefore, deciphering the features conferring specificity to enhancers is important for modulatory gene expression. See, Ernst et al., Nature, 473, 43-49, 2011; Farh et al., Nature, 518, 337-343, 2015; Hnisz et al., Cell, 155, 934-947, 2013; Maurano et al., Science, 337, 1190-1195, 2012, which are hereby incorporated by reference in their entirety. Studies suggest that some of the specificity of enhancer-gene interactions may be due to the interaction of DNA binding transcription factors at enhancers with specific partner transcription factors at promoters. See, Butler and Kadonaga, Genes & Development, 15, 2515-2519, 2001; Choi and Engel, Cell, 55, 17- 26, 1988; Ohtsuki et al., Genes & Development, 12, 547-556, 1998, which are hereby incorporated by reference in their entireties. DNA sequences in enhancers and in promoter-proximal regions bind to a variety of transcription factors expressed in a single cell. Diverse factors bound at these two sites interact with large cofactor complexes and interact with one another to produce enhancer-gene specificity. See, Zabidi et al., Nature, 518:556-559, 2015, which is hereby incorporated by reference in its entirety.

In some embodiments, enhancer regions may be targeted to alter or elucidate gene signaling networks (GSNs).

### Insulators

The term “insulator”, as used herein, refers to regulatory elements that block the ability of an enhancer to activate a gene when located between them and contribute to specific enhancer-gene interactions. See, Chung et al., Cell 74:505-514, 1993; Geyer and Corces, Genes & Development 6:1865-1873, 1992; Kellum and Schedl, Cell 64:941-950, 1991; Udvardy et al., Journal of molecular biology 185:341-358, 1985, which are hereby incorporated by reference in their entirety. Insulators are bound by the transcription factor CTCF but not all CTCF sites function as insulators. See, Bell et al., Cell 98: 387-396, 1999; Liu et al., Nature biotechnology 33:198-203, 2015, which are hereby incorporated by reference in their entireties. The features that distinguish the subset of CTCF sites that function as insulators have not been previously understood.

Genome-wide maps of the proteins that bind enhancers, promoters and insulators, together with knowledge of the physical contacts that occur between these elements provide further insight into understanding of the mechanisms that generate specific enhancer-gene interactions. See, Chepelev et al., Cell research, 22:490-503, 2012; DeMare et al., Genome Research, 23:1224-1234, 2013; Dowen et al., Cell, 159:374-387, 2014; Fullwood et al., Genes & Development 6:1865-1873, 2009; Handoko et al., Nature genetics 43:630-638, 2011; Phillips-Cremins et al., Cell, 153:1281-1295, 2013; Tang et al., Cell 163:1611-1627, 2015, which are hereby incorporated by reference in their entirety Enhancer-bound proteins are constrained such that they tend to interact only with genes within these CTCF-CTCF loops. The subset of CTCF sites that form these loop anchors thus function to insulate enhancers and genes within the loop from enhancers and genes outside the loop, as shown in FIG. 2B. In some embodiments, insulator regions may be targeted to alter or elucidate gene signaling networks (GSNs).

### Cohesin and CTCF Associated Loops and Anchor Sites/Regions

CTCF interactions link sites on the same chromosome forming loops, which are generally less than 1 Mb in length. Transcription occurs both within and outside the loops, but the nature of this transcription differs between the two regions. Studies show that enhancer-associated transcription is more prominent within the loops. Thus, the insulator state is enriched specifically at the CTCF loop anchors. CTCF loops thus either enclose gene poor regions, with a tendency for genes to be centered within the loops or leave out gene dense regions outside the CTCF loops. CTCF loops exhibit reduced exon density relative to their flanking regions. Gene ontology analysis reveals that genes located within CTCF loops are enriched for response to stimuli and for extracellular, plasma membrane and vesicle cellular localizations. On the other hand, genes present within the flanking regions just outside the loops exhibit an expression pattern similar to housekeeping genes i.e. these genes are on average more highly expressed than the loop-enclosed genes, are less cell-line specific in their expression pattern and have less variation in their expression levels across cell lines. See Oti et al., BMC Genomics, 17:252, 2016, which is incorporated by reference in its entirety.

Anchor regions are binding sites for CTCF that influence conformation of an insulated neighborhood. Deletion of anchor sites may result in activation of genes that are usually transcriptionally silent, thereby resulting in a disease phenotype. In fact, somatic mutations are common in loop anchor sites of oncogene-associated insulated neighborhoods. The CTCF DNA-binding motif of the loop anchor region has been observed to be the most altered human transcription-factor binding sequence of cancer cells. See, Hnisz et al., Cell 167, Nov. 17, 2016, which is incorporated by reference in its entirety.

Anchor regions have been observed to be largely maintained during cell development and are especially conserved in the germline of humans and primates. In fact, the DNA sequence of anchor regions are more conserved in CTCF anchor regions than at CTCF binding sites that are not part of an insulated neighborhood. Therefore, cohesin may be used as a target for ChIA-PET to identify locations of both.

Cohesin also becomes associated with CTCF-bound regions of the genome, and some of these cohesin-associated CTCF sites facilitate gene activation while others may function as insulators. See, Dixon et al., Nature, 485(7398):376-80, 2012; Parelho et al., Cell, 132(3):422-33, 2008; Phillips-Cremins and Corces, Molecular Cell, 50(4):461-74, 2013); Seitan et al., Genome Research, 23(12):2066-77, 2013; Wendt et al., Nature, 451(7180):796-801, 2008), which are hereby incorporated by reference in their entireties. Cohesin and CTCF are associated with large loop substructures within TADs, and cohesin and Mediator are associated with smaller loop structures that form within CTCF-bounded regions. See, de Wit et al., Nature, 501(7466):227-31, 2013; Cremins et al., Cell, 153(6):1281-95, 2013; Sofueva et al., EMBO, 32(24):3119-29, 2013, which are hereby incorporated by reference in their entireties. In some embodiments, cohesin and CTCF associated loops and anchor sites/regions may be targeted to alter or elucidate gene signaling networks (GSNs).

### Genetic Variants

Genetic variations within signaling centers are known to contribute to disease by disrupting protein binding on chromosomes, such as described in Hnisz et al., Cell 167, Nov. 17, 2016. Variations of the sequence of CTCF anchor regions that interfere with formation of insulated neighborhoods are observed to result in dysregulation of gene activation and repression. CTCF malfunctions caused by various genetic and epigenetic mechanisms may lead to pathogenesis. Therefore, in some embodiments, it is beneficial to alter any one or more gene signaling networks (GSNs) associated with such variant-driven etiology in order to effect one or more positive treatment outcomes.

### Single Nucleotide Polymorphisms (SNPs)

Most disease associated SNPs are located in the proximity of signaling centers. For example, 94.2% of SNPs occur in non-coding regions, which include signaling centers. In some embodiments, SNPs are altered in order to study and/or alter the signaling from one or more GSN.

### Signaling Molecules

Signaling molecules include any protein that functions in cellular signaling pathways, whether canonical or the gene signaling network pathways defined herein or capable of being defined using the methods described herein. Transcription factors are a subset of signaling molecules. Certain combinations of signaling and master transcription factors associate to an enhancer region to influence expression of a gene. Master regulator factors direct transcription factors in specific tissues. For example, in blood, GATA transcription factors are master regulators that direct TCF7L2 of the Wnt cellular signaling pathway. In the liver, HNG4 is a master regulator to direct SMAD in lineage tissues and patterns.

Transcriptional regulation allows controlling how often a given gene is transcribed. Transcription factors alter the rate at which transcripts are produced by making conditions for transcription initiation more or less favorable. A transcription factor selectively alters a signaling pathway which in turn affects the genes expressed by a signaling center. Signaling centers are components of transcriptional regulators. In some embodiments, signaling molecules may be used, targeted in order to elucidate or alter the signaling of gene signaling networks of the present disclosure.

Table 18 of U.S. 62/501,795, which is hereby incorporated by reference in its entirety, provides a list of signaling molecules including those which act as transcription factors (TF) and/or chromatin remodeling factors (CR) that function in various cellular signaling pathways. The methods described herein may be used to inhibit or activate the expression of one or more signaling molecules associated with the regulatory sequence region of the primary neighborhood gene encoded within an insulated neighborhood. The methods may thus alter the signaling signature of one or more primary neighborhood genes which are differentially expressed upon treatment with the therapeutic agent compared to an untreated control.

### Transcription Factors

The term “transcription factors”, as used herein, refers to signaling molecules which alter, whether to increase or decrease, the transcription of a target gene, e.g., a neighborhood gene. Transcription factors generally regulate gene expression by binding to enhancers and recruiting coactivators and RNA polymerase II to target genes. See Whyte et al., Cell, 153(2): 307-319, 2013, which is incorporated by reference in its entirety. Transcription factors bind “enhancers” to stimulate cell-specific transcriptional program by binding regulatory elements distributed throughout the genome.

There are about 1800 known transcription factors in the human genome. There are epitopes on the DNA of the chromosomes that provide binding sites for proteins or nucleic acid molecules such as ribosomal RNA complexes. Master regulators direct a combination of transcription factors through cell signaling above and DNA below. These characteristics allow for determination of the location of the next signaling center. In some embodiments, transcription factors may be used or targeted, to alter or elucidate the gene signaling networks of the present disclosure.

### Master Transcription Factors

Master transcription factors bind and establish cell-type specific enhancers. Master transcription factors recruit additional signaling proteins, such as other transcription factors, to enhancers to form signaling centers. An atlas of candidate master TFs for 233 human cell types and tissues is described in D'Alessio et al., Stem Cell Reports 5, 763-775 (2015), which is hereby incorporated by reference in its entirety. In some embodiments, master transcription factors may be used or targeted, to alter or elucidate the gene signaling networks of the present disclosure.

### Signaling Transcription Factors

Signaling transcription factors are transcription factors, such as homeoproteins, that travel between cells as they contain protein domains that allow them to do the so. Homeoproteins such as Engrailed, Hoxa5, Hoxb4, Hoxc8, Emx1, Emx2, Otx2 and Pax6 are able to act as signaling transcription factors. The homeoprotein Engrailed possesses internalization and secretion signals that are believed to be present in other homeoproteins as well. This property allows homeoproteins to act as signaling molecules in addition to being transcription factors. Homeoproteins lack characterized extracellular functions leading to the perception that their paracrine targets are intracellular. The ability of homeoproteins to regulate transcription and, in some cases, translation is most likely to affect paracrine action. See Prochiantz and Joliot, Nature Reviews Molecular Cell Biology, 2003. In some embodiments, signaling transcription factors may be used or targeted, to alter or elucidate the gene signaling networks of the present disclosure.

### Chromatin Modifications

Chromatin remodeling is regulated by over a thousand proteins that are associated with histone modification. See, Ji et al., PNAS, 112(12):3841-3846(2015), which is hereby incorporated by reference in its entirety. Chromatin regulators are specific sets of proteins associated with genomic regions marked with modified histones. For example, histones may be modified at certain lysine residues: H3K20me3, H3K27ac, H3K4me3, H3K79me2, H3K36me3, H3K9me2, and H3K9me3. Certain histone modifications mark regions of the genome that are available for binding by signaling molecules. For example, previous studies have observed that active enhancer regions include nucleosomes with H3K27ac, and active promoters include nucleosomes with H3K27ac. Further, transcribed genes include nucleosomes with H3K79me2. ChIP-MS may be performed to identify chromatin regulator proteins associated with specific histone modification. ChIP-seq with antibodies specific to certain modified histones may also be used to identify regions of the genome that are bound by signaling molecules. In some embodiments, chromatin modifying enzymes or proteins may be used or targeted, to alter or elucidate the gene signaling networks of the present disclosure.

RNAs Derived from Regulatory Sequence Regions

Many active regulatory sequence regions (RSRs), such as enhancers, signaling centers, and promoters of protein-coding genes, are known to produce non-coding RNAs.

Transcripts produced at or in the vicinity of active regulatory sequence regions have been implicated in transcription regulation of nearby genes. Recent reports have demonstrated that enhancer-associated RNAs (eRNAs) are strong indicators of enhancer activity (See Li et al., Nat Rev Genet. 2016 April; 17(4):207-23, which is hereby incorporated by reference in its entirety). Further, non-coding RNAs from active regulatory sequence regions have been shown to be involved in facilitating the binding of transcription factors to these regions (Sigova et al., Science. 2015 Nov. 20; 350(6263):978-81, which is hereby incorporated by reference in its entirety). This suggests that such RNAs may be important for the assembly of signaling centers and regulation of neighborhood genes. In some embodiments, RNAs derived from regulatory sequence regions of the target gene may be used or targeted to alter or elucidate the gene signaling networks of the present disclosure.

In some embodiments, RNAs derived from regulatory sequence regions may be an enhancer-associated RNA (eRNA). In some embodiments, RNAs derived from regulatory sequence regions may be a promoter-associated RNA, including but not limited to, a promoter upstream transcript (PROMPT), a promoter-associated long RNA (PALR), and a promoter-associated small RNA (PASR). In further embodiments, RNAs derived from regulatory sequence regions may include but are not limited to transcription start sites (TSS)-associated RNAs (TSSa-RNAs), transcription initiation RNAs (tiRNAs), and terminator-associated small RNAs (TASRs).

In some embodiments, RNAs derived from regulatory sequence regions may be long non-coding RNAs (lncRNAs) (i.e., >200 nucleotides). In some embodiments, RNAs derived from regulatory sequence regions may be intermediate non-coding RNAs. (i.e., about 50 to 200 nucleotides). In some embodiments, RNAs derived from regulatory sequence regions may be short non-coding RNAs (i.e., about 20 to 50 nucleotides).

In some embodiments, eRNAs that may be modulated by methods and compounds described herein may be characterized by one or more of the following features: (1) transcribed from regions with high levels of monomethylation on lysine 4 of histone 3 (H3K4me1) and low levels of trimethylation on lysine 4 of histone 3 (H3K4me3); (2) transcribed from genomic regions with high levels of acetylation on lysine 27 of histone 3 (H3K27ac); (3) transcribed from genomic regions with low levels of trimethylation on lysine 36 of histone 3 (H3K36me3); (4) transcribed from genomic regions enriched for RNA polymerase II (Pol II); (5) transcribed from genomic regions enriched for transcriptional co-regulators, such as the p300 co-activator; (6) transcribed from genomic regions with low density of CpG island; (7) their transcription is initiated from Pol II-binding sites and elongated bidirectionally; (8) evolutionarily conserved DNA sequences encoding eRNAs; (9) short half-life; (10) reduced levels of splicing and polyadenylation, (11) dynamically regulated upon signaling; (12) positively correlated to levels of nearby mRNA expression; (13) extremely high tissue specificity; (14) preferentially nuclear and chromatin-bound; and/or (15) degraded by the exosome.

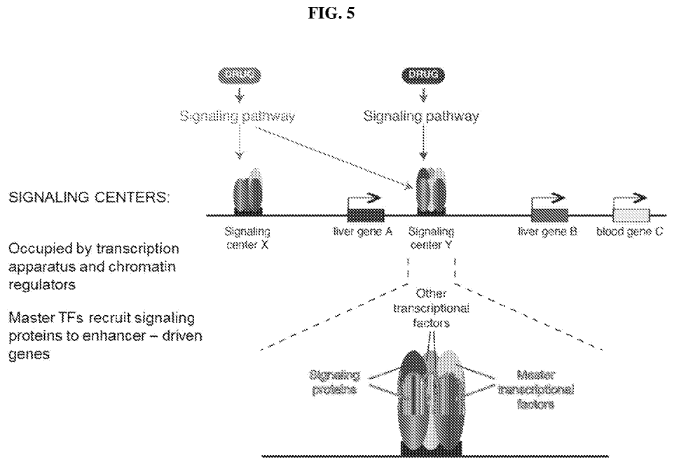

Non-limiting examples of eRNAs that may be modulated by methods and compounds described herein include those described in Djebali et al., Nature. 2012 Sep. 6; 489(7414) (for example, Supplementary data file for FIG. 5a) and Andersson et al., Nature. 2014 Mar. 27; 507(7493):455-461 (for example, Supplementary Tables S3, S12, S13, S15, and 16), which are herein incorporated by reference in their entirety.

In some embodiments, promoter-associated RNAs that may be modulated by methods or compounds described herein may be characterized by one or more of the following features: (1) transcribed from regions with high levels of H3K4me1 and low to medium levels of H3K4me3; (2) transcribed from genomic regions with high levels of H3K27ac; (3) transcribed from genomic regions with no or low levels of H3K36me3; (4) transcribed from genomic regions enriched for RNA polymerase II (Pol II); (5) transcribed from genomic regions with high density of CpG island; (6) their transcription is initiated from Pol II-binding sites and elongated in the opposite direction from the sense strand (that is, mRNAs) or bidirectionally; (7) short half-life; (8) reduced levels of splicing and polyadenylation; (9) preferentially nuclear and chromatin-bound; and/or (10) degraded by the exosome.

Methods and compositions described herein may be used to modulate RNAs derived from regulatory sequence regions to alter or elucidate the gene signaling networks of the present disclosure. In some embodiments, methods and compounds described herein may be used to inhibit the production and/or function of an RNA derived from regulatory sequence regions. In some embodiments, a hybridizing oligonucleotide such as an siRNA or an antisense oligonucleotide may be used to inhibit the activity of the RNA of interest via RNA interference (RNAi), or RNase H-mediated cleavage, or physically block binding of various signaling molecules to the RNA. Exemplary hybridizing oligonucleotide may include those described in U.S. Pat. No. 9,518,261 and WO2014/040742, which are hereby incorporated by reference in their entirety. The hybridizing oligonucleotide may be provided as a chemically modified or unmodified RNA, DNA, locked nucleic acids (LNA), or a combination of RNA and DNA, a nucleic acid vector encoding the hybridizing oligonucleotide, or a virus carrying such vector. In other embodiments, genome editing tools such as CRISPR/Cas9 may be used to delete specific DNA elements in the regulatory sequence regions that control the transcription of the RNA or degrade the RNA itself. In other embodiments, genome editing tools such as a catalytically inactive CRISPR/Cas9 may be used to bind to specific elements in the regulatory sequence regions and block the transcription of the RNA of interest. In further embodiments, bromodomain and extra-terminal domain (BET) inhibitors (e.g., JQ1, I-BET) may be used to reduce RNA transcription through inhibition of histone acetylation by BET protein Brd4.

In some embodiments, methods and compounds described herein may be used to increase the production and/or function of an RNA derived from regulatory sequence regions. In some embodiments, an exogenous synthetic RNA that mimic the RNA of interest may be introduced into the cell. The synthetic RNA may be provided as an RNA, a nucleic acid vector encoding the RNA, or a virus carrying such vector. In other embodiments, genome editing tools such as CRISPR/Cas9 may be used to tether an exogenous synthetic RNA to specific sites in the regulatory sequence regions. Such RNA may be fused to the guide RNA of the CRISPR/Cas9 complex.

In some embodiments, modulation of RNAs derived from regulatory sequence regions increases the expression of a target gene. In some embodiments, modulation of RNAs derived from regulatory sequence regions reduces the expression of the target gene.

In some embodiments, RNAs modulated by compounds described herein include RNAs derived from regulatory sequence regions of the target gene in a liver cell (e.g., hepatocytes).

### Perturbation of Genomic Systems

Behavior of one or more components of the gene signaling networks (GSNs), genomic signaling centers (GSCs), and/or insulated neighborhoods (INs) related to a target gene as described herein may be altered by contacting the systems containing such networks, centers and/or neighborhoods with a perturbation stimulus. Potential stimuli may include exogenous biomolecules such as small molecules, antibodies, proteins, peptides, lipids, fats, nucleic acids, and the like or environmental stimuli such as radiation, pH, temperature, ionic strength, sound, light and the like.

The present disclosure serves, not only as a discovery tool for the elucidation of better defined gene signaling networks (GSNs) and consequently a better understanding of biological systems. The present disclosure allows the ability to properly define gene signaling at the gene level in a manner which allows the prediction, a priori, of potential treatment outcomes, the identification of novel compounds or targets which may have never been implicated in the treatment of a genetic disease, disorder, or condition, reduction or removal of one or more treatment liabilities associated with new or known drugs such as toxicity, poor half-life, poor bioavailability, lack of or loss of efficacy or pharmacokinetic or pharmacodynamic risks.

Treatment of disease by altering gene expression of canonical cellular signaling pathways has been shown to be effective. Even small changes in gene expression may have a significant impact on disease. For example, changes in signaling centers leading to signaling pathways affecting cell suicide suppression are associated with disease. The present disclosure, by elucidating a more definitive set of connectivities of the GSNs provides a fine-tuned mechanism to address disease, including genetic diseases. A method of treating a disease may include modifying a signaling center that is involved in a gene associated with that disease. Such genes may not presently be associated with the disease except as is elucidated using the methods described herein.

A perturbation stimulus may be a small molecule, a known drug, a biological, a vaccine, an herbal preparation, a hybridizing oligonucleotide (e.g., siRNA and antisense oligonucleotide), a gene or cell therapy product, or other treatment product.

In some embodiments, methods of the present disclosure include applying a perturbation stimulus to perturb GSNs, genomic signaling centers, and/or insulated neighborhoods associated with the target gene. Perturbation stimuli that cause changes in target gene expression may inform the connectivities of the associated GSNs and provide potential targets and/or treatments for a related disease, disorder, or condition.

### Downstream Targets

In certain embodiments, a stimulus is administered that targets a downstream product of a gene of a gene signaling network. Alternatively, the stimulus disrupts a gene signaling network that affects downstream expression of at least one downstream target. In some embodiments, the gene is one listed in Table 1.

mRNA

Perturbation of a single or multiple gene signaling network (GSN) associated with a single insulated neighborhood or across multiple insulated neighborhoods can affect the transcription of a single gene or a multiple set of genes by altering the boundaries of the insulated neighborhood due to loss of anchor sites comprising cohesins. Perturbation stimuli may result in the modification of the RNA expression and/or the sequences in the primary transcript within the mRNA, i.e. the exons or the RNA sequences between the exons that are removed by splicing, i.e. the introns. Such changes may consequently alter the members of the set of signaling molecules within the gene signaling network of a gene, thereby defining a variant of the gene signaling network.

### Proteins

Perturbation of a single or multiple gene signaling networks associated with a single insulated neighborhood or across multiple insulated neighborhoods can affect the translation of a single gene or a multiple set of genes that are part of the genomic signaling center, as well as those downstream to the genomic signaling center. Perturbation might result in the inhibition of the translated protein.

### Nearest Neighbor Gene

Perturbation stimuli may cause interactions with signaling molecules to occur in order to alter expression of the nearest primary neighborhood gene that may be located upstream or downstream of the primary neighborhood gene. Neighborhood genes may have any number of upstream or downstream genes along the chromosome. Within any insulated neighborhood, there may be one or more, e.g., one, two, three, four or more, upstream and/or downstream neighborhood genes relative to the primary neighborhood gene. A “primary neighborhood gene” is a gene which is most commonly found within a specific insulated neighborhood along a chromosome. An upstream neighborhood gene of a primary neighborhood gene may be located within the same insulated neighborhood as the primary neighborhood gene. A downstream neighborhood gene of a primary neighborhood gene may be located within the same insulated neighborhood as the primary neighborhood gene.

### Additional Definitions

The term “analog,” as used herein, refers to a compound that is structurally related to the reference compound and shares a common functional activity with the reference compound.

The term “biologic,” as used herein, refers to a medical product made from a variety of natural sources such as micro-organism, plant, animal, or human cells.

The term “boundary,” as used herein, refers to a point, limit, or range indicating where a feature, element, or property ends or begins.

The term “compound,” as used herein, refers to a single agent or a pharmaceutically acceptable salt thereof, or a bioactive agent or drug.

The term “derivative,” as used herein, refers to a compound that differs in structure from the reference compound, but retains the essential properties of the reference molecule.

The term “downstream neighborhood gene,” as used herein, refers to a gene downstream of primary neighborhood gene that may be located within the same insulated neighborhood as the primary neighborhood gene.

The term “gene,” as used herein, refers to a unit or segment of the genomic architecture of an organism, e.g., a chromosome. Genes may be coding or non-coding. Genes may be encoded as contiguous or non-contiguous polynucleotides. Genes may be DNA or RNA.

The term “genomic system architecture,” as used herein, refers to the organization of an individual's genome and includes chromosomes, topologically associating domains (TADs), and insulated neighborhoods.

The term “master transcription factor,” as used herein, refers to signaling molecules which alter, whether to increase or decrease, the transcription of a target gene, e.g., a neighborhood gene and establish cell-type specific enhancers. Master transcription factors recruit additional signaling proteins, such as other transcription factors to enhancers to form signaling centers.

The term “modulate,” as used herein, refers to an alteration (e.g., increase or decrease) in the expression of the target gene and/or activity of the gene product.

The term “neighborhood gene,” as used herein, refers to a gene localized within an insulated neighborhood.

The term “penetrance,” as used herein, refers to the proportion of individuals carrying a particular variant of a gene (e.g., mutation, allele or generally a genotype, whether wild type or not) that also exhibits an associated trait (phenotype) of that variant gene and in some situations is measured as the proportion of individuals with the mutation who exhibit clinical symptoms thus existing on a continuum.

The term “polypeptide,” as used herein, refers to a polymer of amino acid residues (natural or unnatural) linked together most often by peptide bonds. The term, as used herein, refers to proteins, polypeptides, and peptides of any size, structure, or function. In some instances, the polypeptide encoded is smaller than about 50 amino acids and the polypeptide is then termed a peptide. If the polypeptide is a peptide, it will be at least about 2, 3, 4, or at least 5 amino acid residues long.

The term “primary neighborhood gene,” as used herein, refers to a gene which is most commonly found within a specific insulated neighborhood along a chromosome.

The term “primary downstream boundary,” as used herein, refers to the insulated neighborhood boundary located downstream of a primary neighborhood gene.

The term “primary upstream boundary,” as used herein, refers to the insulated neighborhood boundary located upstream of a primary neighborhood gene.

The term “promoter,” as used herein, refers to a DNA sequence that defines where transcription of a gene by RNA polymerase begins and defines the direction of transcription indicating which DNA strand will be transcribed.

The term “regulatory sequence regions,” as used herein, include but are not limited to regions, sections or zones along a chromosome whereby interactions with signaling molecules occur in order to alter expression of a neighborhood gene.

The term “repressor,” as used herein, refers to any protein that binds to DNA and therefore regulates the expression of genes by decreasing the rate of transcription.

The term “secondary downstream boundary,” as used herein, refers to the downstream boundary of a secondary loop within a primary insulated neighborhood.

The term “secondary upstream boundary,” as used herein, refers to the upstream boundary of a secondary loop within a primary insulated neighborhood.

The term “signaling molecule,” as used herein, refers to any entity, whether protein, nucleic acid (DNA or RNA), organic small molecule, lipid, sugar or other biomolecule, which interacts directly, or indirectly, with a regulatory sequence region on a chromosome.

The term “signaling transcription factor,” as used herein, refers to signaling molecules which alter, whether to increase or decrease, the transcription of a target gene, e.g., a neighborhood gene and also act as cell-cell signaling molecules.

The term “small molecule,” as used herein, refers to a low molecular weight drug, i.e. <5000 Daltons organic compound that may help regulate a biological process.

The terms “subject” and “patient,” are used interchangeably herein and refer to an animal to whom treatment with the compositions according to the present disclosure is provided.

The term “therapeutic agent,” as used herein, refers to a substance that has the ability to cure a disease or ameliorate the symptoms of the disease.

The term “therapeutic or treatment outcome,” as used herein, refers to any result or effect (whether positive, negative or null) which arises as a consequence of the perturbation of a GSC or GSN. Examples of therapeutic outcomes include, but are not limited to, improvement or amelioration of the unwanted or negative conditions associated with a disease or disorder, lessening of side effects or symptoms, cure of a disease or disorder, or any improvement associated with the perturbation of a GSC or GSN.

The term “therapeutic or treatment liability,” as used herein, refers to a feature or characteristic associated with a treatment or treatment regime which is unwanted, harmful or which mitigates the therapies positive outcomes. Examples of treatment liabilities include for example toxicity, poor half-life, poor bioavailability, lack of or loss of efficacy or pharmacokinetic or pharmacodynamic risks.

The term “upstream neighborhood gene,” as used herein, refers to a gene upstream of a primary neighborhood gene that may be located within the same insulated neighborhood as the primary neighborhood gene.

### Canonical Cell Signaling Pathways

It is understood that there may, and most likely will, be some overlap between the canonical pathways detailed in the art and the gene signaling networks (GSNs) defined herein.

Whereas canonical pathways permit a certain degree of promiscuity of members across pathways (cross talk), gene signaling networks (GSN) of the disclosure are defined at the gene level and characterized based on any number of stimuli or perturbation to the cell, tissue, organ or organ system expressing that gene. Hence the nature of a GSN is both structurally (e.g., the gene) and situationally (e.g., the function, e.g., expression profile) defined. And while two different gene signaling networks may share members, they are still unique in that the nature of the perturbation can distinguish them. Hence, the value of gene signaling networks in the elucidation of the function of biological systems in support of therapeutic research and development.

It should be understood that it is not intended that no connection ever be made between canonical pathways and gene signaling networks; in fact, the opposite is the case. In order to bridge the two signaling paradigms for further scientific insights, it will be instructive to compare the canonical signaling pathway paradigm with the gene signaling networks of the present disclosure.

In some embodiments, methods of the present disclosure involve altering the Janus kinases (JAK)/signal transducers and activators of transcription (STAT) pathway. The JAK/STAT pathway is the major mediator for a wide array of cytokines and growth factors. Cytokines are regulatory molecules that coordinate immune responses. JAKs are a family of intracellular, nonreceptor tyrosine kinases that are typically associated with cell surface receptors such as cytokine receptors. Mammals are known to have 4 JAKs: JAK1, JAK2, JAK3, and Tyrosine kinase 2 (TYK2). Binding of cytokines or growth factors to their respective receptors at the cell surface initiates trans-phosphorylation of JAKs, which activates downstream STATs. STATs are latent transcription factors that reside in the cytoplasm until activated. There are seven mammalian STATs: STAT1, STAT2, STAT3, STAT4, STAT5 (STAT5A and STAT5B), and STAT6. Activated STATs translocate to the nucleus where they complex with other nuclear proteins and bind to specific sequences to regulate the expression of target genes. Thus, the JAK/STAT pathway provides a direct mechanism to translate an extracellular signal into a transcriptional response. Target genes regulated by JAK/STAT pathway are involved in immunity, proliferation, differentiation, apoptosis and oncogenesis. Activation of JAKs may also activate the phosphatidylinositol 3-kinase (PI3K) and mitogen-activated protein kinase (MAPK) pathways.

In some embodiments, methods of the present disclosure involve altering the mitogen-activated protein kinase (MAPK) signaling pathway. The MAPK pathway involves a chain of signaling molecules (e.g., Ras, Raf, MEK, and ERK) in the cell that communicates a signal from a receptor at the cell membrane to the nucleus. This pathway can be activated by receptor-linked tyrosine kinases such as epidermal growth factor receptor (EGFR), Trk A/B, Fibroblast growth factor receptor (FGFR) and PDGFR. The MAPK signaling pathway is essential in regulating numerous cellular processes including cell stress response, cell differentiation, cell division, cell proliferation, inflammation, metabolism, motility and apoptosis. MAPK interacts with major pathway targets: ERK1/2, ERK5, JNK, and p38 kinase. MAPK regulates the activities of several transcription factors including C-myc, CREB and C-Fos. MAPK also interacts with other pathways such as the PI3K networks, NF-κB and JAK/STAT pathways.

In some embodiments, methods of the present disclosure involve altering the Platelet-derived Growth Factor Receptor (PDGFR)-mediated signal pathway. PDGFRs are cell surface tyrosine kinase receptors for members of the platelet-derived growth factor (PDGF) family There are two isoforms of PDGFRs, PDGFRα and PDGFRβ. The two receptor isoforms dimerize upon binding the PDGF dimer, leading to the activation of the kinase. PDGFRs mediate a number of signaling pathways that are important for regulating cell proliferation, cellular differentiation, cell growth and development. Inhibition of the PDGFR-mediated signaling pathway has been correlated with reduced expression of PDGF, ang1/2, and VEGF mRNA. Since PDGF is a known stimulus for PI3-K activation, inhibiting PDGFR may lead to decreased activation of the PI3-K signaling cascade. The role of PDGFs and PDGFRs in physiology and medicine is reviewed in Andrae et al., Genes Dev. 2008 May 15; 22(10):1276-312, which is hereby incorporated by reference in its entirety.

Other canonical pathways which may also be altered according to the present disclosure include, but are not limited to the 2-arachidonoylglycerol biosynthesis pathway, 2-oxocarboxylic acid metabolism pathway, 5HT1 type receptor mediated signaling pathway, 5HT2 type receptor mediated signaling pathway, 5HT3 type receptor mediated signaling pathway, 5HT4 type receptor mediated signaling pathway, 5-hydroxytryptamine biosynthesis pathway, 5-hydroxytryptamine degradation pathway, abacavir transport and metabolism pathway, ABC transporters pathway, ABC-family proteins mediated transport pathway, ACE inhibitor pathway, acetate utilization pathway, acetylcholine synthesis pathway, activation of camp-dependent PKA pathway, activin beta signaling pathway, adenine and hypoxanthine salvage pathway, adherens junction pathway, adipocytokine signaling pathway, adipogenesis pathway, adrenaline and noradrenaline biosynthesis pathway, adrenergic signaling in cardiomyocytes pathway, advanced glycation end-products (age/rage) pathway, advanced glycosylation end product receptor signaling pathway, aflatoxin bl metabolism pathway, age/rage pathway, AHR pathway, AKT signaling pathway, alanine and aspartate metabolism pathway, alanine biosynthesis pathway, aldosterone synthesis and secretion pathway, aldosterone-regulated sodium reabsorption pathway, allantoin degradation pathway, allograft rejection pathway, all-trans-retinoic acid signaling pathway, alp23b signaling pathway, alpha 6 beta 4 signaling pathway, alpha adrenergic receptor signaling pathway, alpha6 beta4 integrin pathway, alpha-linolenic acid metabolism pathway, Alzheimer disease-amyloid secretase pathway, Alzheimer disease-presenilin pathway, amino acid conjugation pathway, amino sugar and nucleotide sugar metabolism pathway, aminoacyl-tRNA biosynthesis pathway, aminobutyrate degradation pathway, AMP-activated protein kinase pathway, AMPK signaling pathway, anandamide biosynthesis pathway, anandamide degradation pathway, androgen receptor signaling pathway, androgen/estrogen/progesterone biosynthesis pathway, angiogenesis pathway, angiopoietin-Tie2 signaling pathway, angiotensin II-stimulated signaling through g proteins and beta-arrestin pathway, antigen processing and presentation by MHC's pathway, apoptosis modulation and signaling pathway, apoptosis modulation by HSP70 pathway, apoptosis signaling pathway, apoptosis through death receptors pathway, apoptotic execution phase pathway, arachidonate epoxygenase/epoxide hydrolase pathway, arachidonic acid metabolism pathway, arginine and proline metabolism pathway, arginine biosynthesis pathway, aripiprazole metabolic pathway, arylamine metabolism pathway, ascorbate and aldarate metabolism pathway, ascorbate degradation pathway, asparagine and aspartate biosynthesis pathway, asparagine N-linked glycosylation pathway, aspartate and glutamate metabolism pathway, assembly of RNA polymerase-II initiation complex pathway, ATM pathway, ATP synthesis pathway, axon guidance pathway, axon guidance mediated by netrin pathway, axon guidance mediated by semaphorins pathway, axon guidance mediated by slit/robo pathway, B cell activation pathway, B cell receptor (BCR) pathway, B cell receptor signaling pathway, bacterial invasion of epithelial cells pathway, basal transcription factors pathway, base excision repair pathway, B-cell development pathway, B-cell receptor pathway, B-cell receptor complex pathway, benzo pathway, beta1 adrenergic receptor signaling pathway, beta2 adrenergic receptor signaling pathway, beta3 adrenergic receptor signaling pathway, beta-alanine metabolism pathway, bile acid and bile salt metabolism pathway, bile secretion pathway, binding and uptake of ligands by scavenger receptors pathway, biogenic amine synthesis pathway, biosynthesis of amino acids pathway, biosynthesis of unsaturated fatty acids pathway, biotin biosynthesis pathway, blakely network pathway, blood clotting cascade pathway, blood coagulation pathway, bmp/activin signaling-drosophila pathway, bone morphogenic protein pathway, brain-derived neurotrophic factor (BDNF) pathway, BRCA1 pathway, bupropion degradation pathway, butanoate metabolism pathway, butirosin and neomycin biosynthesis pathway, butyrate-induced histone acetylation pathway, cadherin signaling pathway, caffeine metabolism pathway, calcium regulation in the cardiac cell pathway, calcium signaling pathway, cAMP pathway, carbohydrate digestion and absorption pathway, carbon metabolism pathway, cardiac muscle contraction pathway, cardiac progenitor differentiation pathway, carnitine metabolism pathway, caspase cascade pathway, catalytic cycle of mammalian flavin-containing monooxygenases pathway, CCKR signaling map pathway, CCR5 in macrophages pathway, CD4 and CD8 T-cell lineage pathway, CD40 signaling pathway, CDK5 pathway, cell adhesion molecules (cams) pathway, cell cycle checkpoints pathway, cell cycle pathway, cell differentiation—meta pathway, cell junction organization pathway, cell surface interactions at the vascular wall pathway, CGMP-PKG signaling pathway, chemical carcinogenesis pathway, chemokine signaling pathway, cholesterol biosynthesis pathway, cholinergic synapse pathway, chorismate biosynthesis pathway, chromatin remodeling pathway, circadian clock system pathway, circadian entrainment pathway, citrate cycle (TCA cycle) pathway, c-met pathway, cobalamin biosynthesis pathway, codeine and morphine metabolism pathway, coenzyme A biosynthesis pathway, coenzyme A linked carnitine metabolism pathway, colchicine metabolic pathway, collecting duct acid secretion pathway, complement and coagulation cascades pathway, cori cycle pathway, corticotropin-releasing hormone (CRH) pathway, costimulation by the CD28 family pathway, CREB pathway, CTL mediated apoptosis pathway, CTLA4 signaling pathway, cyanoamino acid metabolism pathway, cyclins and cell cycle regulation pathway, cysteine and methionine metabolism pathway, cysteine biosynthesis pathway, cytokine network pathway, cytokine-cytokine receptor interaction pathway, cytokines and inflammatory response pathway, cytoplasmic ribosomal proteins pathway, cytoskeletal regulation by rho GTPase pathway, cytosolic DNA-sensing pathway, de novo purine biosynthesis pathway, de novo pyrimidine deoxyribonucleotide biosynthesis pathway, de novo pyrimidine ribonucleotides biosynthesis pathway, depolarization of the presynaptic terminal triggers the opening of calcium channels pathway, diclofenac metabolic pathway, differentiation pathway, digestion resistant carbohydrate metabolism pathway, dilated cardiomyopathy pathway, dissolution of fibrin clot pathway, diurnally regulated genes with circadian orthologs pathway, div no colors pathway, div pathway, DNA damage bypass pathway, DNA damage response pathway, DNA damage reversal pathway, DNA methylation and transcriptional repression pathway, DNA repair mechanisms pathway, DNA replication pathway, dopamine metabolism pathway, dopamine receptor mediated signaling pathway, dopaminergic synapse pathway, dorso-ventral axis formation pathway, DPP signaling pathway, DPP-SCW signaling pathway, drug metabolism pathway, drug metabolism—cytochrome p450 pathway, dscam interactions pathway, E2F/MIRHG1 feedback-loop—delete pathway, EBV LMP1 signaling pathway, ECM-receptor interaction pathway, effects of nitric oxide pathway, effects of pip2 hydrolysis pathway, EGF pathway, EGF receptor signaling pathway, eicosanoid synthesis pathway, electron transport chain pathway, endochondral ossification pathway, endocrine and other factor-regulated calcium reabsorption pathway, endocytosis pathway, endoderm differentiation pathway, endogenous cannabinoid signaling pathway, endothelin pathway, endothelin signaling pathway, energy metabolism pathway, enkephalin release pathway, enos signaling pathway, ephrin-EPHR signaling pathway, epidermal growth factor receptor (EGFR) pathway, epithelial cell signaling in helicobacter pylori infection pathway, epithelial tight junctions pathway, EPO receptor signaling pathway, ERBB signaling pathway, ERK signaling pathway, erythropoietin pathway, estrogen signaling pathway, ether lipid metabolism pathway, eukaryotic transcription initiation pathway, eukaryotic translation elongation pathway, eukaryotic translation initiation pathway, eukaryotic translation termination pathway, FAK1 signaling pathway, Fas signaling pathway, fat digestion and absorption pathway, fatty acid pathway, fatty acid beta oxidation pathway, fatty acid biosynthesis pathway, fatty acid degradation pathway, fatty acid elongation pathway, fatty acid metabolism pathway, fatty acid omega oxidation pathway, FGF pathway, FGF signaling pathway, fibroblast growth factor-1 (FGF1) pathway, flavin biosynthesis pathway, FLT3 signaling pathway, fluoropyrimidine activity pathway, focal adhesion pathway, folate biosynthesis pathway, folate metabolism pathway, follicle stimulating hormone pathway, formation of fibrin clot pathway, formyltetrahydroformate biosynthesis pathway, Foxo signaling pathway, fructose galactose metabolism pathway, G protein signaling pathway, G1 to S cell cycle control pathway, G13 signaling pathway, GABA synthesis pathway, GABA-B receptor II signaling pathway, galactose metabolism pathway, gamma-aminobutyric acid synthesis pathway, ganglio sphingolipid metabolism pathway, gap junction trafficking and regulation pathway, gastric acid secretion pathway, gastrin pathway, GBB signaling pathway, generic transcription pathway, ghrelin pathway, glial cell differentiation pathway, globo sphingolipid metabolism pathway, glucagon signaling pathway, glucocorticoid & mineralcorticoid metabolism pathway, glucocorticoid receptor signaling pathway, glucose homeostasis pathway, glucuronidation pathway, glutamatergic synapse pathway, glutamine glutamate conversion pathway, glutathione metabolism pathway, glycan degradation pathway, glycerolipid metabolism pathway, glycerophospholipid biosynthetic pathway, glycerophospholipid metabolism pathway, glycine metabolism pathway, glycogen metabolism pathway, glycolysis/gluconeogenesis pathway, glycosaminoglycan biosynthesis-heparan sulfate/heparin pathway, glycosaminoglycan biosynthesis-keratan sulfate pathway, glycosaminoglycan degradation pathway, glycosaminoglycan metabolism pathway, glycosphingolipid biosynthesis—ganglio series pathway, glycosphingolipid biosynthesis-globo series pathway, glycosphingolipid biosynthesis—lacto and neolacto series pathway, glyoxylate and dicarboxylate metabolism pathway, gonadotropin-releasing hormone receptor pathway, GP1B-IX-V activation signaling pathway, GPCR pathway, GPCR downstream signaling pathway, GPCR ligand binding pathway, GPVI-mediated activation cascade pathway, granulocyte adhesion and diapedesis pathway, granzyme pathway, growth hormone signaling pathway, GSK 3 signaling pathway, hedgehog signaling pathway, hematopoiesis from pluripotent stem cells pathway, hematopoietic cell lineage pathway, hematopoietic stem cell differentiation pathway, heme biosynthesis pathway, hepatitis B pathway, hepatitis C pathway, heterotrimeric G-protein signaling-Gi alpha and Gs alpha mediated pathway, heterotrimeric g-protein signaling-rod outer segment phototransduction pathway, hexose transport pathway, HGF pathway, HIF-1 signaling pathway, hippo signaling pathway, histamine h1 receptor mediated signaling pathway, histamine h2 receptor mediated signaling pathway, histamine synthesis pathway, histidine biosynthesis pathway, histone modifications pathway, homologous recombination pathway, HTLV-I infection pathway, human complement system pathway, hypoxia response via hif activation pathway, ID signaling pathway, IGF1R signaling pathway, IL1 and megakaryocytes in obesity pathway, IL-1 signaling pathway, IL-10 pathway, IL17 signaling pathway, IL-2 signaling pathway, IL-22 pathway, IL-3 signaling pathway, IL-4 signaling pathway, IL-5 signaling pathway, IL-6 pathway, IL-7 signaling pathway, IL-9 signaling pathway, ILK signaling pathway, inflammation mediated by chemokine and cytokine signaling pathway, inflammatory mediator regulation of Trp channels pathway, inflammatory response pathway, influenza a virus infection pathway, inos signaling pathway, inositol phosphate metabolism pathway, insulin receptor pathway, insulin resistance pathway, insulin secretion pathway, insulin/IGF-protein kinase b signaling cascade pathway, insulin-like growth factor-2 mRNA binding proteins pathway, integrin alphaIIb beta3 signaling pathway, integrin cell signaling pathway, integrin cell surface interactions pathway, integrin-mediated cell adhesion pathway, interferon pathway, interferon alpha/beta signaling pathway, interferon type I signaling pathway, interferon-gamma signaling pathway, interleukin signaling pathway, interleukin-1 (IL-1) pathway, interleukin-1 processing pathway, interleukin-11 signaling pathway, interleukin-2 (IL-2) pathway, interleukin-3 pathway, interleukin-4 (IL-4) pathway, interleukin-5 (IL-5) pathway, interleukin-6 (IL-6) pathway, interleukin-7 (IL-7) pathway, interleukin-9 (il-9) pathway, intracellular calcium signaling pathway, ionotropic glutamate receptor pathway, IP3 pathway, isoleucine biosynthesis pathway, JAK/STAT pathway, JNK pathway, kinesins pathway, KIT receptor pathway, LDL oxidation in atherogenesis pathway, leptin (lep) pathway, leptin signaling pathway, leucine biosynthesis pathway, leukocyte transendothelial migration pathway, linoleic acid metabolism pathway, lipid digestion pathway, lipoate_biosynthesis pathway, longevity regulating—mammal pathway, longevity regulating—multiple species pathway, long-term potentiation pathway, lysine biosynthesis pathway, lysine degradation pathway, lysosome pathway, mannose metabolism pathway, MAPK cascade pathway, MAPK targets/nuclear events mediated by MAP kinases pathway, matrix metalloproteinases pathway, melatonin metabolism and effects pathway, meta biotransformation pathway, metabolism of carbohydrates pathway, metabolism of nitric oxide pathway, metabolism of nucleotides pathway, metabolism of porphyrins pathway, metabolism of water-soluble vitamins and cofactors pathway, metabolism of xenobiotics by cytochrome p450 pathway, metabotropic glutamate receptor group I pathway, metabotropic glutamate receptor group II pathway, metabotropic glutamate receptor group III pathway, methionine biosynthesis pathway, methylation pathway, methylcitrate cycle pathway, methylmalonyl pathway, mineral absorption pathway, miRNA biogenesis pathway, mismatch repair pathway, mitochondrial apoptosis pathway, mitochondrial gene expression pathway, mitochondrial lc-fatty acid beta-oxidation pathway, mitotic G1-G1/S phases pathway, mitotic G2-G2/M phases pathway, monoamine GPCRs pathway, monoamine transport pathway, mRNA capping pathway, mRNA editing pathway, mRNA processing pathway, mRNA splicing pathway, mRNA surveillance pathway, mTOR signaling pathway, muscarinic acetylcholine receptor 1 and 3 signaling pathway, muscarinic acetylcholine receptor 2 and 4 signaling pathway, myogenesis pathway, myometrial relaxation and contraction pathway, N-acetylglucosamine metabolism pathway, NAD biosynthesis II pathway, nanomaterial induced apoptosis pathway, nanoparticle triggered autophagic cell death pathway, nanoparticle triggered regulated necrosis pathway, natural killer cell mediated cytotoxicity pathway, ncam signaling for neurite out-growth pathway, nephrin interactions pathway, netrin-1 signaling pathway, neural crest differentiation pathway, neuroactive ligand-receptor interaction pathway, neurotransmitter clearance in the synaptic cleft pathway, neurotransmitter release cycle pathway, neurotransmitter uptake and metabolism in glial cells pathway, neurotrophin signaling pathway, NFAT and cardiac hypertrophy pathway, NF-kappa b signaling pathway, NF-kappa b signaling pathway, NGF pathway, NGF signaling via TRKA from the plasma membrane pathway, N-glycan biosynthesis pathway, nicotinate and nicotinamide metabolism pathway, nicotine activity on chromaffin cells pathway, nicotine activity on dopaminergic neurons pathway, nicotine degradation pathway, nicotine metabolism pathway, nicotine pharmacodynamics pathway, nicotinic acetylcholine receptor signaling pathway, nifedipine activity pathway, nitrogen metabolism pathway, NLR proteins pathway, nod-like receptor signaling pathway, non-homologous end joining pathway, notch signaling pathway, Nrf2 pathway, nuclear receptors pathway, nucleosome assembly pathway, nucleotide excision repair pathway, nucleotide GPCRs pathway, nucleotide metabolism pathway, nucleotide-binding oligomerization domain pathway, o-antigen biosynthesis pathway, o-glycan biosynthesis pathway, olfactory transduction pathway, oncostatin m signaling pathway, one carbon metabolism pathway, opioid prodynorphin pathway, opioid proenkephalin pathway, opioid proopiomelanocortin pathway, ornithine degradation pathway, osteoblast signaling pathway, osteoclast signaling pathway, osteopontin signaling pathway, ovarian steroidogenesis pathway, oxidation by cytochrome p450 pathway, oxidative phosphorylation pathway, oxidative stress pathway, oxytocin receptor mediated signaling pathway, oxytocin signaling pathway, p38 MAPK signaling pathway, p53 feedback loops 1 pathway, p53 feedback loops 2 pathway, p53 mediated apoptosis pathway, p53 signaling pathway, pak pathway, pancreatic secretion pathway, pantothenate biosynthesis pathway, parkin-ubiquitin proteasomal system pathway, passive transport by aquaporins pathway, PDGF signaling pathway, pentose and glucuronate interconversions pathway, pentose phosphate pathway, peptide GPCRs pathway, peptidoglycan biosynthesis pathway, peroxisomal beta-oxidation of tetracosanoyl-coA pathway, peroxisomal lipid metabolism pathway, pertussis pathway, phagosome pathway, phase 1-functionalization of compounds pathway, phase I biotransformations pathway, phase II conjugation pathway, phenylacetate degradation pathway, phenylalanine biosynthesis pathway, phenylalanine metabolism pathway, phenylethylamine degradation pathway, phenylpropionate degradation pathway, phosphatidylinositol signaling system pathway, phospholipase D signaling pathway, phototransduction pathway, PI3 kinase pathway, PI3K signaling in B-lymphocytes pathway, PI3K-AKT signaling pathway, PIP3 activates AKT signaling pathway, plasminogen activating cascade pathway, platelet activation pathway, platelet adhesion to exposed collagen pathway, platelet aggregation pathway, platelet homeostasis pathway, polyol pathway, porphyrin and chlorophyll metabolism pathway, PPAR signaling pathway, primary bile acid biosynthesis pathway, primary focal segmental glomerulosclerosis FSGs pathway, processing of capped intron-containing pre-mRNA pathway, processing of capped intronless pre-mRNA pathway, progesterone-mediated oocyte maturation pathway, prolactin signaling pathway, proline biosynthesis pathway, propanoate metabolism pathway, prostaglandin synthesis and regulation pathway, proteasome pathway, proteasome degradation pathway, protein digestion and absorption pathway, protein export pathway, protein folding pathway, proximal tubule bicarbonate reclamation pathway, PRPP biosynthesis pathway, PTEN pathway, purine metabolism pathway, pyridoxal phosphate salvage pathway, pyridoxal-5-phosphate biosynthesis pathway, pyrimidine metabolism pathway, pyruvate metabolism pathway, rac1 pathway, rank signaling in osteoclast pathway, rank1/rank pathway, rap1 signaling pathway, ras signaling pathway, Ras-RAF-MEK-ERK pathway, receptor activator of nuclear factor kappa-b ligand (RANKL) pathway, regulation of actin cytoskeleton pathway, regulation of apoptosis pathway, regulation of autophagy pathway, regulation of DNA replication pathway, regulation of lipolysis in adipocytes pathway, regulation of microtubule cytoskeleton pathway, regulation of toll-like receptor signaling pathway, remodeling of adherens junctions pathway, renin secretion pathway, renin-angiotensin system pathway, respiratory electron transport pathway, retinol metabolism pathway, retrograde endocannabinoid signaling pathway, Rho family GTPase pathway, Rhoa pathway, ribosome biogenesis in eukaryotes pathway, RIG-I-like receptor signaling pathway, RNA degradation pathway, RNA polymerase I pathway, RNA polymerase II transcription pathway, RNA transport pathway, RNAi pathway, s-adenosylmethionine biosynthesis pathway, salivary secretion pathway, salvage pyrimidine deoxyribonucleotides pathway, salvage pyrimidine ribonucleotides pathway, SCW signaling pathway, selenium metabolism and selenoproteins pathway, selenium micronutrient network pathway, selenocompound metabolism pathway, semaphorin interactions pathway, serine and threonine metabolism pathway, serine glycine biosynthesis pathway, serotonergic synapse pathway, serotonin htr1 group and fos pathway, serotonin receptor 2 and ELK-SRF/gata4 signaling pathway, serotonin receptor 2 and STAT3 signaling pathway, serotonin receptor 4/6/7 and NR3C signaling pathway, serotonin transporter activity pathway, signal amplification pathway, signal regulatory protein pathway, signal transduction of S1P receptor pathway, signaling by EGFR pathway, signaling by insulin receptor pathway, signaling by PDGF pathway, signaling by rho GTPases pathway, signaling by robo receptor pathway, signaling by VEGF pathway, signaling in gap junction pathway, signaling of hepatocyte growth factor receptor pathway, signaling regulating pluripotency of stem cells pathway, signaling in glioblastoma pathway, signaling by NGF pathway, SMAD signaling network pathway, small ligand GPCRs pathway, SNARE interactions in vesicular transport pathway, sphingolipid (SM) signaling pathway, sphingolipid metabolism pathway, spliceosome pathway, starch and sucrose metabolism pathway, stat signaling pathway, STAT3 pathway, statin pathway, steroid biosynthesis pathway, steroid hormone biosynthesis pathway, sterol regulatory element-binding proteins pathway, striated muscle contraction pathway, succinate to proprionate conversion pathway, sulfate assimilation pathway, sulfation biotransformation reaction pathway, sulfur metabolism pathway, sulfur relay system pathway, sumo pathway, synaptic vesicle pathway, synthesis and degradation of ketone bodies pathway, synthesis of DNA pathway, T cell receptor (TCR) pathway, tamoxifen metabolism pathway, tarbase pathway, target of rapamycin pathway, taste transduction pathway, taurine and hypotaurine metabolism pathway, TCA and urea cycles pathway, T-cell antigen receptor pathway, T-cell receptor and co-stimulatory signaling pathway, telomere maintenance pathway, terpenoid backbone biosynthesis pathway, tetrahydrofolate biosynthesis pathway, TFS regulate miRNAs related to cardiac hypertrophy pathway, TGF-beta pathway, TGF-beta receptor signaling pathway, THC differentiation pathway, thiamin biosynthesis pathway, thiamin metabolism pathway, threonine biosynthesis pathway, thymic stromal lymphopoietin pathway, thymic stromal lymphopoietin (tslp) pathway, thyroid-stimulating hormone (tsh) pathway, thyrotropin-releasing hormone receptor signaling pathway, tie2/tek signaling pathway, tight junction pathway, TNF alpha signaling pathway, TNF related weak inducer of apoptosis pathway, TNF signaling pathway, TNF superfamily pathway, TNF-related weak inducer of apoptosis (tweak) pathway, toll receptor signaling pathway, toll-like receptors pathway, TP53 network pathway, Traf pathway, trail pathway, transcription regulation by bzip transcription factor pathway, transcriptional activation by Nrf2 pathway, transendothelial migration of leukocytes pathway, transforming growth factor beta (TGF-beta) receptor pathway, translation factors pathway, transmission across electrical synapses pathway, transport of glucose and other sugars pathway, transport of glycerol from adipocytes to the liver by aquaporins pathway, transport of vitamins pathway, trans-sulfuration pathway, trans-sulfuration and one carbon metabolism pathway, triacylglyceride synthesis pathway, triacylglycerol metabolism pathway, tRNA aminoacylation pathway, tryptophan biosynthesis pathway, tryptophan metabolism pathway, tumor necrosis factor (TNF) alpha pathway, tumoricidal effects of hepatic NK cells pathway, tweak pathway, type II diabetes mellitus pathway, type II interferon signaling pathway, type III interferon signaling pathway, tyrosine and tryptophan biosynthesis pathway, tyrosine biosynthesis pathway, tyrosine metabolism pathway, ubiquinone and other terpenoid-quinone biosynthesis pathway, ubiquitin mediated proteolysis pathway, ubiquitin proteasome pathway, unfolded protein response pathway, urea cycle and metabolism of amino groups pathway, valine biosynthesis pathway, vascular smooth muscle contraction pathway, vasopressin synthesis pathway, vasopressin-regulated water reabsorption pathway, VEGF signaling pathway, vitamin a and carotenoid metabolism pathway, vitamin b12 metabolism pathway, vitamin b6 biosynthesis pathway, vitamin b6 metabolism pathway, vitamin d metabolism pathway, vitamin digestion and absorption pathway, Wnt signaling pathway, xanthine and guanine salvage pathway, and/or the zinc homeostasis pathway.

### II. DISEASES WITH UNMET NEEDS

In some embodiments, the disease, disorder, or condition may be selected from those having unmet treatment needs. Table 1 provides examples of diseases with unmet treatment needs and proposed genes to target for treatment.

TABLE 1
Diseases with unmet needs

| | Target gene(s) for | Prevalence and | |
|---|---|---|---|
| Disease Name | treatment | Inheritance pattern | Current Standard of Care |
| Fibronectin | FN1 | Ultra-Rare, autosomal | Dialysis, Renal transplant |
| Glomerulopathy | | dominant | |
| Hereditary | CPOX | Rare, autosomal dominant | Hemin; Pain management |
| coproporphyria | | | |
| SERPINC1 | SERPINC1 | Rare, autosomal dominant | Replacement therapy |
| Deficiency | | | |
| Alagille Syndrome | JAG1, NOTCH2 | Rare, autosomal dominant | Symptomatic, Liver transplant |
| Glycogen Storage | SLC37A4 | Rare, autosomal recessive | Prevent hypoglycemia, Symptomatic |
| disease 1b | | | relief |
| Acute Intermittent | HMBS | Rare, autosomal dominant | Panhematin/hemin; pain management, |
| porphyria | | | Liver Transplant |
| LECT2 Amyloidosis | LECT2 | Rare, autosomal recessive | Symptom management, dialysis, |
| | | | kidney transplant |
| APOL1-associated | APOL1 | Rare, autosomal recessive | Symptomatic, dialysis |
| glomerular disease | | | |
| Gilbert Syndrome; | UGT1A1 | Common, benign | Awareness and symptom management |
| Criggler Najjar, type | | | |
| II | | | |
| Dyslipidaemia | Combination: | Common | Diet modification, statins, |
| | PCSK9, | | management |
| | ANGPTL3, LDLR | | |
| Rett Syndrome | MeCP2 | | |

In some embodiments, the disease with an unmet need is sickle cell disease (SCD), which is a severe, rare hematologic disease with limited treatment options. SCD has a large orphan indication of about 100,000 patients in the U.S. and about 60,000 patients in Europe. The disease causes devastating morbidity and mortality of a 2-3 decade reduction in life expectancy resulting from vaso-occlusion, hemolytic anemia, inflammation/vascular injury leading to multi-organ failure. Specifically, in the brain, strokes (infarct or hemorrhage) may occur causing paralysis, neurocognitive deficits, or death. Specifically, in the lungs, acute chest syndrome, pulmonary hypertension, and/or pneumonia may occur. Specifically, in the kidney, hematuria, renal insufficiency, and/or renal failure may occur. Specifically, in the bones and joints, bone marrow infarcts, osteomyelitis, and avascular necrosis/osteonecrosis may occur. Specifically, in the liver/gallbladder, hepatopathy, gallstones, and/or liver failure may occur. Specifically, in the eye, hemorrhage, blindness, retinal detachment, and/or retinopathy may occur. Specifically, in the heart, cardiomegaly and/or heart failure may occur. Specifically, in the spleen, atrophy (autosplenectomy) may occur. Specifically, on the skin, stasis ulcers of ankles and/or dactylitis may occur. Specifically, in men, priapism may occur. Specifically, in women, adverse pregnancy outcomes may occur.

Current treatments include L-glutamine and hydroxyurea. Leading drugs presently in the therapeutic pipeline for SCD include tricagrelor, Sel-G1, GBT-440, and LentiGlobin. HIF stailizers, trichosic, HDAC-1/2 inhibitors, PRMT-5 inhibitors, EdX-17, LSD-1 inhibitor, MBD inhibitors, PB-04, and panobinostat have shown promise for HbF induction for treatment of SCD. Aes-107, PNQ-103, MX-1520, and SCD-101 have shown promise as anti-sickling agents for treatment of SCD. PF-4447943 has shown promise as an anti-adhesion agent for treatment of SCD. VBP-15 and NKTT-120 have shown promise as an anti-inflammatory for treatment of SCD. In some embodiments, the methods herein provide a treatment of SCD by modulating the signaling center and/or insulated neighborhood using at least one of the compounds provided above or at least one stimulus selected from Tables 19-26, 28 of U.S. 62/501,795, which is hereby incorporated by reference in its entirety. In some embodiments, the selected compound or stimulus targets at least one gene selected from Tables 1-9 of U.S. 62/501,795, which is hereby incorporated by reference in its entirety, resulting in rescue of the phenotype of SCD.

### III. COMPOSITIONS AND METHODS

In some embodiments, the present disclosure provides compositions and methods for modulating the expression of one or more targer genes, such as those listed in Table 1. The compositions and methods described herein may be used to treat or prevent a disease, disorder or condition associated with the target gene(s). In some embodiments, the disease, disorder or condition associated with the target gene(s) is one listed in Table 1.

The terms “subject” and “patient” are used interchangeably herein and refer to an animal to whom treatment with the compositions according to the present disclosure is provided. In some embodiments, the subject is a mammal. In some embodiments, the subject is a human being.

In some embodiments, subjects may have been diagnosed with or have symptoms for a disease, disorder or condition associated with one or more target genes. In other embodiments, subjects may be susceptible to or at risk for a disease, disorder or condition associated with one or more target genes.

In some embodiments, subjects may carry one or more mutations within or near the target gene. In some embodiment, subjects may carry one functional allele and one mutated allele of the target gene. In some embodiment, subjects may carry two mutated alleles of the target gene. The mutation(s) may alter the levels or the activity of the protein produced from the target gene.

In some embodiments, subjects may have a deficiency of the protein produced from a target gene compared to a healthy subject. This may be due to mutations that impair protein activity, reduce protein stability, or decrease the expression of the gene. Accordingly, compositions and methods described herein may be used to increase the expression of the target gene to rescue the phenotype of the associated disease, disorder or condition. In other embodiments, subjects may have excessive production of a protein, or production of a protein with unwanted activities, fom a target gene compared to a healthy subject. This may be caused by gain-of-function mutations, impaired degradation process, or misregulated expression. Accordingly, compositions and methods described herein may be used to decrease the expression of the target gene to rescue the phenotype of the associated disease, disorder or condition.

In some embodiments, compositions and methods of the present disclosure may be used to alter the expression of a target gene in a cell. In some embodiments, the cell is a mammalian cell. In some embodiments, the cell is a human cell. In some embodiments, the cell is a mouse cell. In some embodiments, the cell is a hepatocyte.

Changes in gene expression may be assessed at the RNA level or protein level by various techniques known in the art and described herein, such as RNA-seq, qRT-PCR, Western Blot, or enzyme-linked immunosorbent assay (ELISA). Changes in gene expression may be determined by comparing the level of target gene expression in the treated cell or subject to the level of expression in an untreated or control cell or subject. In some embodiments, compositions and methods of the present disclosure cause an increase in the expression of a target gene by at least about 10%, at least about 20%, at least about 30%, at least about 40%, at least about 50%, at least about 60%, at least about 70%, at least about 80%, at least about 90%, at least about 100%, at least about 125%, at least about 150%, at least about 175%, at least about 200%, at least about 250%, at least about 300%, at least about 400%, at least about 500%, from about 25% to about 50%, from about 40% to about 60%, from about 50% to about 70%, from about 60% to about 80%, from about 80% to about 100%, from about 100% to about 125%, from about 100 to about 150%, from about 150% to about 200%, from about 200% to about 300%, from about 300% to about 400%, from about 400% to about 500%, or more than 500%. In some embodiments, compositions and methods of the present disclosure cause a fold change in the expression of a target gene by about 2 fold, about 3 fold, about 4 fold, about 5 fold, about 6 fold, about 7 fold, about 8 fold, about 9 fold, about 10 fold, about 12 fold, about 15 fold, about 18 fold, about 20 fold, about 25 fold, or more than 30 fold. In some embodiments, compositions and methods of the present disclosure cause reduction in the expression of a target gene by at least about 10%, at least about 20%, at least about 30%, at least about 40%, at least about 50%, from about 25% to about 50%, from about 40% to about 60%, from about 50% to about 70%, from about 60% to about 80%, more than 80%, or even more than 90%, 95% or 99%.

In some embodiments, the increase in the expression of a target gene induced by compositions and methods of the present disclosure may be sufficient to prevent or alleviate one or more signs or symptoms of the associated disease, disorder or condition in a subject. In some embodiments, the reduction in the expression of a target gene induced by compositions and methods of the present disclosure may be sufficient to prevent or alleviate one or more signs or symptoms of the associated disease, disorder or condition in a subject. In some embodiments, the changes in the expression of a set of genes induced by compositions and methods of the present disclosure may be sufficient to prevent or alleviate one or more signs or symptoms of the associated disease, disorder or condition in a subject.

In some embodiments, the present disclosure provides compositions and methods for treating or preventing fibronectin glomerulopathy, which is caused by the deposition of fibronectin, encoded by the FN1 gene on chromosome 2q35. In some embodiments, at least one compound or method taught herein reduces the levels of fibronectin by altering the signaling center(s) responsible for controlling the expression of the FN1 gene. The reduction in fibronectin levels may be sufficient to rescue the phenotype of fibronectin glomerulopathy. In certain embodiments, the compound capable of reducing FN1 expression is selected from smoothened agonist, Crizotinib, BGJ398, AZD2858, amlodipine besylate, PHA-665752, OSU-03012, bms-986094 (inx-189), afatinib, LDN193189, sotrastaurin, SKL2001, tivozanib, cedirandib, calcitriol, rimonabant, merestinib, BMP4, and GDF2 (BMP9). In some embodiments, smoothened agonist perturbs at least one component in the Hedgehog/Smoothened pathway to reduce the expression of FN1. In some embodiments, Crizotinib perturbs at least one component in the c-MET pathway to reduce the expression of FN1. In some embodiments, BGJ398 perturbs at least one component in the FGFR pathway to reduce the expression of FN1. In some embodiments, AZD2858 perturbs at least one component in the GSK-3 pathway to reduce the expression of FN1. In some embodiments, amlodipine besylate perturbs at least one component in the Calcium channel pathway to reduce the expression of FN1. In some embodiments, PHA-665752 perturbs at least one component in the c-MET pathway to reduce the expression of FN1. In some embodiments, OSU-03012 perturbs at least one component in the PDK-1 pathway to reduce the expression of FN1. In some embodiments, afatinib perturbs at least one component in the EGFR pathway to reduce the expression of FN1. In some embodiments, LDN193189 perturbs at least one component in the TGF-B pathway to reduce the expression of FN1. In some embodiments, sotrastaurin perturbs at least one component in the PKC pathway to reduce the expression of FN1. In some embodiments, SKL2001 perturbs at least one component in the WNT pathway to reduce the expression of FN1. In some embodiments, tivozanib perturbs at least one component in the Protein Tyrosine Kinase/RTK pathway to reduce the expression of FN1. In some embodiments, cediranib perturbs at least one component in the Protein Tyrosine Kinase/RTK pathway to reduce the expression of FN1. In some embodiments, calcitriol perturbs at least one component in the Vitamin D Receptor pathway to reduce the expression of FN1. In some embodiments, rimonabant perturbs at least one component in the Cannabinoid receptor pathway to reduce the expression of FN1. In some embodiments, merestinib perturbs at least one component in the c-MET pathway to reduce the expression of FN1. In some embodiments, BMP4 perturbs at least one component in the TGF-B pathway to reduce the expression of FN1. In some embodiments, GDF2 (BMP9) perturbs at least one component in the TGF-B pathway to reduce the expression of FN1.

In some embodiments, the present disclosure provides compositions and methods for treating or preventing hereditary coproporphyria, which is caused by a deficiency of the enzyme coproporphyrinogen oxidase, encoded by the CPOX gene on chromosome 3q11.2. In some embodiments, at least one compound or method taught herein increases levels of coproporphyrinogen oxidase by altering the signaling center(s) responsible for controlling the expression of the CPOX gene. The increase in the levels of coproporphyrinogen oxidase may be sufficient to rescue the phenotype of hereditary coproporphyria. In certain embodiments, the compound capable of increasing CPOX expression is selected from thalidomide, Glycopyrrolate, MK-0752, Bosutinib, Nefazodone, Corticosterone, Deferoxamine mesylate, GZD824 Dimesylate, XMU-MP-1, prednisone, FICZ, SKL2001, Cobalt chloride, and 17-AAG (Tanespimycin). In some embodiments, thalidomide perturbs at least one component in the NF-kB pathway to increase the expression of CPOX. In some embodiments, Glycopyrrolate perturbs at least one component in the Acetylcholine receptor pathway to increase the expression of CPOX. In some embodiments, MK-0752 perturbs at least one component in the NOTCH signaling pathway to increase the expression of CPOX. In some embodiments, Bosutinib perturbs at least one component in the Src pathway to increase the expression of CPOX. In some embodiments, Nefazodone perturbs at least one component in the Calcium signaling pathway to increase the expression of CPOX. In some embodiments, Corticosterone perturbs at least one component in the Mineralcorticoid receptor pathway to increase the expression of CPOX. In some embodiments, Deferoxamine mesylate perturbs at least one component in the Hypoxia activated pathway to increase the expression of CPOX. In some embodiments, GZD824 Dimesylate perturbs at least one component in the ABL pathway to increase the expression of CPOX. In some embodiments, XMU-MP-1 perturbs at least one component in the Hippo pathway to increase the expression of CPOX. In some embodiments, prednisone perturbs at least one component in the GR signaling pathway to increase the expression of CPOX. In some embodiments, FICZ perturbs at least one component in the Aryl hydrocarbon receptor pathway to increase the expression of CPOX. In some embodiments, SKL2001 perturbs at least one component in the WNT pathway to increase the expression of CPOX. In some embodiments, Cobalt chloride perturbs at least one component in the Hypoxia activated pathway to increase the expression of CPOX. In some embodiments, 17-AAG (Tanespimycin) perturbs at least one component in the Cell Cycle/DNA Damage; Metabolic Enzyme/Protease pathway to increase the expression of CPOX.

In some embodiments, the present disclosure provides compositions and methods for treating or preventing SERPINC1 deficiency, which is caused by a deficiency of antithrombin (previously known as antithrombin III), encoded by the SERPINC1 gene on chromosome 1q25.1. In some embodiments, at least one compound or method taught herein increases the levels of antithrombin by altering the signaling center(s) responsible for controlling the expression of the SERPINC1 gene. The increase in the levels of antithrombin may be sufficient to rescue the phenotype of SERPINC1 deficiency. In certain embodiments, the compound capable of increasing SERPINC1 expression is selected from CP-673451, echinomycin, pacritinib, amuvatinib, crenolanib, INNO-206 (aldoxorubicin), momelotinib, thalidomide, and pifithrin-μ. In some embodiments, CP-673451 perturbs at least one component in the PDGFR pathway to increase the expression of SERPINC1. In some embodiments, echinomycin perturbs at least one component in the Hypoxia activated pathway to increase the expression of SERPINC1. In some embodiments, pacritinib perturbs at least one component in the JAK-STAT pathway to increase the expression of SERPINC1. In some embodiments, amuvatinib perturbs at least one component in the PDGFR pathway to increase the expression of SERPINC1. In some embodiments, crenolanib perturbs at least one component in the PDGFR pathway to increase the expression of SERPINC1. In some embodiments, INNO-206 (aldoxorubicin) perturbs at least one component in the Cell Cycle/DNA Damage pathway to increase the expression of SERPINC1. In some embodiments, momelotinib perturbs at least one component in the JAK/STAT pathway to increase the expression of SERPINC1. In some embodiments, thalidomide perturbs at least one component in the NF-kB pathway to increase the expression of SERPINC1. In some embodiments, pifithrin-μ perturbs at least one component in the p53 pathway to increase the expression of SERPINC1.

In some embodiments, the present disclosure provides compositions and methods for treating or preventing Alagille Syndrome, which is caused by a deficiency in the jagged 1 ligand and/or Notch2 receptor, encoded by the JAG1 gene on chromosome 20p12.2 and the NOTCH2 gene on chromosome 1p12, respectively. Most patients with Alagille Syndrome have haploinsufficiency in jagged 1, and some also deficiency in Notch2. In some embodiments, at least one compound or method taught herein increases the levels of jagged 1 and Notch2 by altering the signaling center(s) responsible for controlling the expression of the JAG1 gene and/or the NOTCH2 gene. The increase in the levels of JAG1 and/or Notch2 may be sufficient to rescue the phenotype of Alagille Syndrome. In certain embodiments, the compound capable of increasing JAG1 and/or NOTCH2 expression is selected from merestinib and torcetrapib to increase expression of both genes. In certain embodiments, the compound is selected from LDN193189, LDN212854, thalidomide, phenformin, enzastaurin, GDF2 (BMP9), BMP2, INNO-206 (aldoxorubicin), amuvatinib, BMP4, and BAY 87-2243 to alter the signaling center(s) for JAG1 to increase expression of JAG1. Alternatively, the compound is selected from zibotentan and 740 Y-P to alter the signaling center(s) for NOTCH2 to increase NOTCH2 expression. In some embodiments, LDN193189 perturbs at least one component in the TGF-B pathway to increase the expression of JAG1 or NOTCH2. In some embodiments, LDN-212854 perturbs at least one component in the TGF-B pathway to increase the expression of JAG1 or NOTCH2. In some embodiments, Thalidomide perturbs at least one component in the NF-kB pathway to increase the expression of JAG1 or NOTCH2. In some embodiments, Phenformin perturbs at least one component in the AMPK pathway to increase the expression of JAG1 or NOTCH2. In some embodiments, Enzastaurin perturbs at least one component in the Epigenetics; TGF-beta/Smad pathway to increase the expression of JAG1 or NOTCH2. In some embodiments, GDF2 (BMP9) perturbs at least one component in the TGF-B pathway to increase the expression of JAG1 or NOTCH2. In some embodiments, BMP2 perturbs at least one component in the TGF-B pathway to increase the expression of JAG1 or NOTCH2. In some embodiments, INNO-206 (aldoxorubicin) perturbs at least one component in the Cell Cycle/DNA Damage pathway to increase the expression of JAG1 or NOTCH2. In some embodiments, Merestinib perturbs at least one component in the c-MET pathway to increase the expression of JAG1 or NOTCH2. In some embodiments, Amuvatinib perturbs at least one component in the PDGFR pathway to increase the expression of JAG1 or NOTCH2. In some embodiments, BMP4 perturbs at least one component in the TGF-B pathway to increase the expression of JAG1 or NOTCH2. In some embodiments, BAY 87-2243 perturbs at least one component in the Hypoxia activated pathway to increase the expression of JAG1 or NOTCH2. In some embodiments, Zibotentan perturbs at least one component in the GPCR/G protein pathway to increase the expression of JAG1 or NOTCH2. In some embodiments, 740 Y-P perturbs at least one component in the PI3K/AKT pathway to increase the expression of JAG1 or NOTCH2.

In some embodiments, the present disclosure provides compositions and methods for treating or preventing glycogen storage disease 1b, which is caused by a deficiency of the glucose-6-phosphate translocase (G6PT), encoded by the gene SLC37A4 on chromosome 11q23.3. Mutations in the coding region SLC37A4 can lead to a partially functional protein. In some embodiments, at least one compound or method taught herein increases the levels of glucose-6-phosphate translocase by altering the signaling center(s) responsible for controlling the expression of the SLC37A4 gene. The increase in the levels of glucose-6-phosphate translocase (G6PT) may be sufficient to rescue the phenotype of glycogen storage disease 1b. In certain embodiments, the compound capable of increasing SLC37A4 expression is selected from echinomycin, prednisone, CP-673451, cobalt chloride, amuvatinib, pacritinib, R788 (fostamatinib, disodium hexahydrate, GZD824 dimesylate, corticosterone, dexamethasone, TNF-α (TNF-a), thalidomide, and IGF-1. In some embodiments, Echinomycin perturbs at least one component in the Hypoxia activated pathway to increase the expression of SLC37A4. In some embodiments, prednisone perturbs at least one component in the GR signaling pathway to increase the expression of SLC37A4. In some embodiments, CP-673451 perturbs at least one component in the PDGFR pathway to increase the expression of SLC37A4. In some embodiments, Cobalt chloride perturbs at least one component in the Hypoxia activated pathway to increase the expression of SLC37A4. In some embodiments, Amuvatinib perturbs at least one component in the PDGFR pathway to increase the expression of SLC37A4. In some embodiments, Pacritinib (SB1518) perturbs at least one component in the JAK-STAT pathway to increase the expression of SLC37A4. In some embodiments, R788 (fostamatinib disodium hexahydrate) perturbs at least one component in the Protein Tyrosine Kinase/RTK pathway to increase the expression of SLC37A4. In some embodiments, GZD824 Dimesylate perturbs at least one component in the ABL pathway to increase the expression of SLC37A4. In some embodiments, Corticosterone perturbs at least one component in the Mineralcorticoid receptor pathway to increase the expression of SLC37A4. In some embodiments, Dexamethasone perturbs at least one component in the Glucocorticoid receptor pathway to increase the expression of SLC37A4. In some embodiments, TNF-a perturbs at least one component in the NF-kB, MAPK, Apoptosis pathway to increase the expression of SLC37A4. In some embodiments, Thalidomide perturbs at least one component in the NF-kB pathway to increase the expression of SLC37A4. In some embodiments, IGF-1 perturbs at least one component in the IGF-1R/InsR pathway to increase the expression of SLC37A4.

In some embodiments, the present disclosure provides compositions and methods for treating or preventing acute intermittent porphyria, which is caused by a deficiency of hydroxymethylbilane synthase (HMBS), encoded by the HMBS gene on chromosome 11q23.3. In some embodiments, at least one compound or method taught herein increases the levels of HMBS by altering the signaling center(s) responsible for controlling the expression of the HMBS gene. The increase in the levels of HMBS may be sufficient to rescue the phenotype of acute intermittent porphyria. In some embodiments, the compound capable of increasing HMBS expression is sotrastaurin. In some embodiments, sotrastaurin perturbs at least one component in the Protein Kinase C (PKC) signaling pathway to increase the expression of HMBS.

In some embodiments, the present disclosure provides compositions and methods for treating or preventing LECT2 amyloidosis, which is caused by the deposition of the Leukocyte Chemotactic Factor 2 (LECT2) protein, encoded by the LECT2 gene on chromosome 5q31.1. In some embodiments, at least one compound or method taught herein decreases the levels of LECT2 by altering the signaling center(s) responsible for controlling the expression of the LECT2 gene. The reduction in the levels of LECT2 may be sufficient to rescue the phenotype of LECT2 amyloidosis. In some embodiments, the compound capable of reducing LECT2 expression is selected from Calcitriol, 17-AAG (Tanespimycin), Ritonavir, TFP, b-Estradiol, Rifampicin, Torcetrapib, Zibotentan, Rimonabant, OSU-03012, Afatinib, NSC228155, Glucose, APS-2-79, Phorbol 1213-dibutyrate, prednisone, 740 Y-P, Amlodipine Besylate, and Darapladib. In some embodiments, calcitriol perturbs at least one component in the Vitamin D Receptor pathway to reduce the expression of LECT2. In some embodiments, 17-AAG (Tanespimycin) perturbs at least one component in the Cell Cycle/DNA Damage; Metabolic Enzyme/Protease pathway to reduce the expression of LECT2. In some embodiments, TFP perturbs at least one component in the P53 pathway to reduce the expression of LECT2. In some embodiments, b-Estradiol perturbs at least one component in the ER pathway to reduce the expression of LECT2. In some embodiments, Rifampicin perturbs at least one component in the PXR pathway to reduce the expression of LECT2. In some embodiments, Zibotentan perturbs at least one component in the GPCR/G protein pathway to reduce the expression of LECT2. In some embodiments, Rimonabant perturbs at least one component in the Cannabinoid receptor pathway to reduce the expression of LECT2. In some embodiments, OSU-03012 perturbs at least one component in the PDK-1 pathway to reduce the expression of LECT2. In some embodiments, Afatinib perturbs at least one component in the EGFR pathway to reduce the expression of LECT2. In some embodiments, NSC228155 perturbs at least one component in the EGFR pathway to reduce the expression of LECT2. In some embodiments, Glucose perturbs at least one component in the metabolic/glycolysis pathway to reduce the expression of LECT2. In some embodiments, APS-2-79 perturbs at least one component in the MAPK pathway to reduce the expression of LECT2. In some embodiments, Phorbol 1213-dibutyrate perturbs at least one component in the PKC pathway to reduce the expression of LECT2. In some embodiments, prednisone perturbs at least one component in the GR pathway to reduce the expression of LECT2. In some embodiments, 740 Y-P perturbs at least one component in the PI3K/AKT pathway to reduce the expression of LECT2. In some embodiments, Amlodipine Besylate perturbs at least one component in the Calcium channel pathway to reduce the expression of LECT2.

In some embodiments, the present disclosure provides compositions and methods for treating or preventing APOL1-associated glomerular disease, which is caused by the risk variants of apolipoprotein L1 (APOL1), encoded by the APOL1 gene on chromosome 22q12.3. In some embodiments, at least one compound or method taught herein decreases the levels of UDP-glycuronosyltransferase by altering the signaling center(s) responsible for controlling the expression of the APOL1 gene. The reduction in the levels of APOL1 may be sufficient to rescue the phenotype of APOL1-associated glomerular disease. In some embodiments, the compound capable of reducing APOL1 expression is selected from nitrofurantoin and crizotinib. In some embodiments, nitrofurantoin perturbs at least one component in the antibiotic pathway to reduce the expression of APOL1. In some embodiments, crizotinib perturbs at least one component in the c-MET pathway to reduce the expression of APOL1.

In some embodiments, the present disclosure provides compositions and methods for treating or preventing Gilbert Syndrome and/or Criggler Najjar, type II, which are caused by decreased activities of uridine 5′-diphosphate(UDP)-glycuronosyltransferase, encoded by the UGT1A1 gene on chromosome 2q37.1. In some embodiments, at least one compound or method taught herein increases the levels of UDP-glycuronosyltransferase by altering the signaling center(s) responsible for controlling the expression of the UGT1A1 gene. The increase in the levels of UDP-glycuronosyltransferase may be sufficient to rescue the phenotype of Gilbert Syndrome and/or Criggler Najjar, type II. In certain embodiments, the compound or stimulus is selected from FICZ, Kartogenin, meBIO, CP-673451, BAM7, EW-7197, Pacritinib (SB1518), Pifithrin-a, LY294002, BMS-754807, Bexarotene, Crizotinib, ARN-509, Echinomycin, JNJ-38877605, Omeprazole, RO4929097, Momelotinib, BIRB 796, AZD6738, Semagacestat, Glimepiride, AZD1480, Cryptotanshinone, GW4064, LRH-1 antagonist, PND-1186, Crenolanib, EB1089, Sotrastaurin, Corticosterone, GZD824 Dimesylate, Netarsudil, R788 (fostamatinib disodium hexahydrate), Oxoglaucine, Evacetrapib, LY2584702, Merestinib, CI-4AS-1, Dasatinib, Rolofylline (KW-3902), IWP-2, T0901317, Ritonavir, BIO, Amuvatinib, FRAX597, Anti mullerian hormone, Wnt3a, Decernotinib, Dorsomorphin, Etomidate, and GDC-0879. In some embodiments, FICZ perturbs at least one component in the Aryl hydrocarbon receptor pathway to increase the expression of UGT1A1. In some embodiments, Kartogenin perturbs at least one component in the TGF-B pathway to increase the expression of UGT1A1. In some embodiments, meBIO perturbs at least one component in the Aryl hydrocarbon receptor pathway to increase the expression of UGT1A1. In some embodiments, CP-673451 perturbs at least one component in the PDGFR pathway to increase the expression of UGT1A1. In some embodiments, BAM7 perturbs at least one component in the BCL2 pathway to increase the expression of UGT1A1. In some embodiments, EW-7197 perturbs at least one component in the TGF-B pathway to increase the expression of UGT1A1. In some embodiments, Pifithrin-a perturbs at least one component in the p53 pathway to increase the expression of UGT1A1. In some embodiments, LY294002 perturbs at least one component in the PI3K/AKT pathway to increase the expression of UGT1A1. In some embodiments, BMS-754807 perturbs at least one component in the IGF-1R/InsR pathway to increase the expression of UGT1A1. In some embodiments, Bexarotene perturbs at least one component in the RXR pathway to increase the expression of UGT1A1. In some embodiments, Crizotinib perturbs at least one component in the c-MET pathway to increase the expression of UGT1A1. In some embodiments, ARN-509 perturbs at least one component in the Androgen receptor pathway to increase the expression of UGT1A1. In some embodiments, Echinomycin perturbs at least one component in the Hypoxia activated pathway to increase the expression of UGT1A1. In some embodiments, JNJ-38877605 perturbs at least one component in the c-MET pathway to increase the expression of UGT1A1. In some embodiments, Omeprazole perturbs at least one component in the Proton pump pathway to increase the expression of UGT1A1. In some embodiments, RO4929097 perturbs at least one component in the NOTCH pathway to increase the expression of UGT1A1. In some embodiments, Momelotinib perturbs at least one component in the JAK/STAT pathway to increase the expression of UGT1A1. In some embodiments, BIRB 796 perturbs at least one component in the MAPK pathway to increase the expression of UGT1A1. In some embodiments, AZD6738 perturbs at least one component in the ATM/ATR pathway to increase the expression of UGT1A1. In some embodiments, Semagacestat perturbs at least one component in the Notch, Neuronal Signaling; Stem Cells/Wnt pathway to increase the expression of UGT1A1. In some embodiments, Glimepiride perturbs at least one component in the Potassium channel pathway to increase the expression of UGT1A1. In some embodiments, AZD1480 perturbs at least one component in the JAK/STAT pathway to increase the expression of UGT1A1. In some embodiments, Cryptotanshinone perturbs at least one component in the JAK/STAT pathway to increase the expression of UGT1A1. In some embodiments, GW4064 perturbs at least one component in the FXR pathway to increase the expression of UGT1A1. In some embodiments, LRH-1 antagonist perturbs at least one component in the LHR-1 pathway to increase the expression of UGT1A1. In some embodiments, PND-1186 perturbs at least one component in the FAK pathway to increase the expression of UGT1A1. In some embodiments, Crenolanib perturbs at least one component in the PDGFR pathway to increase the expression of UGT1A1. In some embodiments, EB1089 perturbs at least one component in the Vitamin D Receptor pathway to increase the expression of UGT1A1. In some embodiments, Sotrastaurin perturbs at least one component in the PKC pathway to increase the expression of UGT1A1. In some embodiments, Corticosterone perturbs at least one component in the Mineralcorticoid receptor pathway to increase the expression of UGT1A1. In some embodiments, GZD824 Dimesylate perturbs at least one component in the ABL pathway to increase the expression of UGT1A1. In some embodiments, Netarsudil perturbs at least one component in the ROCK pathway to increase the expression of UGT1A1. In some embodiments, R788 (fostamatinib disodium hexahydrate) perturbs at least one component in the Protein Tyrosine Kinase/RTK pathway to increase the expression of UGT1A1. In some embodiments, Oxoglaucine perturbs at least one component in the PI3K/AKT pathway to increase the expression of UGT1A1. In some embodiments, LY2584702 perturbs at least one component in the S6K pathway to increase the expression of UGT1A1. In some embodiments, Merestinib perturbs at least one component in the c-MET pathway to increase the expression of UGT1A1. In some embodiments, CI-4AS-1 perturbs at least one component in the Androgen receptor pathway to increase the expression of UGT1A1. In some embodiments, Dasatinib perturbs at least one component in the ABL pathway to increase the expression of UGT1A1. In some embodiments, IWP-2 perturbs at least one component in the WNT pathway to increase the expression of UGT1A1. In some embodiments, T0901317 perturbs at least one component in the LXR pathway to increase the expression of UGT1A1. In some embodiments, BIO perturbs at least one component in the Pan-GSK-3 pathway to increase the expression of UGT1A1. In some embodiments, Amuvatinib perturbs at least one component in the PDGFR pathway to increase the expression of UGT1A1. In some embodiments, FRAX597 perturbs at least one component in the PAK pathway to increase the expression of UGT1A1. In some embodiments, Anti mullerian hormone perturbs at least one component in the TGF-B pathway to increase the expression of UGT1A1. In some embodiments, Wnt3a perturbs at least one component in the WNT pathway to increase the expression of UGT1A1. In some embodiments, Decernotinib perturbs at least one component in the JAK/STAT pathway to increase the expression of UGT1A1. In some embodiments, Dorsomorphin perturbs at least one component in the AMPK pathway to increase the expression of UGT1A1. In some embodiments, Etomidate perturbs at least one component in the GABAergic receptor pathway to increase the expression of UGT1A1. In some embodiments, GDC-0879 perturbs at least one component in the MAPK pathway to increase the expression of UGT1A1.

In some embodiments, the present disclosure provides compositions and methods for treating or preventing dyslipidemia, which has been associated with defects in low density lipoprotein receptor (LDLR), gain of function mutations in proprotein convertase subtilisin/kexin type 9 (PCSK9), and increased expression of angiopoietin like 3 (ANGPTL3). LDLR is encoded by the LDLR gene on chromosome 19p13.2; PCSK9 is encoded by the PCSK9 gene on chromosome 1p32.3; and ANGPTL3 is encoded by the ANGPTL3 gene on chromosome 1p31.3. In some embodiments, at least one compound or method taught herein increases the levels of LDL receptor and/or decreases the level of PCSK9 and/or ANGPTL3 by altering the signaling center(s) responsible for controlling the expression of the LDLR, PCSK9 and/or ANGPTL3. The increase in the levels of LDL receptor and/or reduction in the levels of PCSK9 and/or ANGPTL3 may be ANGPTL3 may be sufficient to rescue the phenotype of dyslipidemia, which includes disorders of lipoprotein metabolism that result in multiple abnormalities, including: high total cholesterol, high LDL-C, or high triglycerides. In certain embodiments, the compound capable of increasing LDLR expression and/or decreasing PCSK9 and/or ANGPTL3 expression is selected from WYE-125132 (WYE-132) and pifithrin-μ. In certain embodiments, the compound is selected from SGI-1776, preladenant, and CO-1686 (rociletinib) to decrease ANGPTL3 and increase LDLR. Alternatively, the compound may be LY294002 to increase LDLR and decrease PCSK9.

In some embodiments, the present disclosure provides compositions and methods for treating or preventing Rett Syndrome, which has been associated with defects in Methyl-CpG Binding Protein 2 (MECP2). MECP2 is encoded by the MECP2 gene on chromosome Xq28. In some embodiments, at least one compound or method taught herein increases the levels of MECP2 by altering the signaling center(s) responsible for controlling the expression of MECP2. The increase in the levels of MECP2 may be sufficient to rescue the phenotype of Rett Syndrome. In certain embodiments, the compound capable of increasing MECP2 expression is 17-AAG (Tanespimycin)/KOS-953.

### Small Molecules

In some embodiments, compounds used to modulate the expression of a target gene may include small molecules. As used herein, the term “small molecule” refers a low molecular weight drug, i.e. <5000 Daltons organic compound that may help regulate a biological process. In some embodiments, small molecule compounds described herein are applied to a genomic system to interfere with components (e.g., transcription factor, signaling proteins) of the gene signaling networks associated with the target gene, thereby modulating the expression of the target gene. In some embodiments, small molecule compounds described herein are applied to a genomic system to alter the boundaries of an insulated neighborhood and/or disrupt signaling centers associated with the target gene, thereby modulating the expression of the target gene.

A small molecule screen may be performed to identify small molecules that act through signaling centers of an insulated neighborhood to alter gene signaling networks which may modulate expression of the target gene. For example, known signaling agonists/antagonists may be administered. Credible hits are identified and validated by the small molecules that are known to work through a signaling center and modulate expression of the target gene.

### Polypeptides

In some embodiments, compounds for altering expression of a target gene comprise a polypeptide. As used herein, the term “polypeptide” refers to a polymer of amino acid residues (natural or unnatural) linked together most often by peptide bonds. The term, as used herein, refers to proteins, polypeptides, and peptides of any size, structure, or function. In some instances, the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analog of a corresponding naturally occurring amino acid.

### Antibodies

In some embodiments, compounds for altering expression of a target gene comprise an antibody. In one embodiment, antibodies of the present disclosure comprising antibodies, antibody fragments, their variants or derivatives described herein are specifically immunoreactive with at least one component of the gene signaling networks associated with the target gene.

As used herein, the term “antibody” is used in the broadest sense and specifically covers various embodiments including, but not limited to monoclonal antibodies, polyclonal antibodies, multispecific antibodies (e.g. bispecific antibodies formed from at least two intact antibodies), and antibody fragments such as diabodies so long as they exhibit a desired biological activity. Antibodies are primarily amino-acid based molecules but may also comprise one or more modifications such as with sugar moieties.

“Antibody fragments” comprise a portion of an intact antibody, preferably comprising an antigen binding region thereof. Examples of antibody fragments include Fab, Fab′, F(ab′)2, and Fv fragments; diabodies; linear antibodies; single-chain antibody molecules; and multispecific antibodies formed from antibody fragments. Papain digestion of antibodies produces two identical antigen-binding fragments, called “Fab” fragments, each with a single antigen-binding site. Also produced is a residual “Fc” fragment, whose name reflects its ability to crystallize readily. Pepsin treatment yields an F(ab′)2 fragment that has two antigen-binding sites and is still capable of cross-linking antigen. Antibodies of the present disclosure may comprise one or more of these fragments. For the purposes herein, an “antibody” may comprise a heavy and light variable domain as well as an Fc region.

“Native antibodies” are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain.

As used herein, the term “variable domain” refers to specific antibody domains that differ extensively in sequence among antibodies and are used in the binding and specificity of each particular antibody for its particular antigen. As used herein, the term “Fv” refers to antibody fragments which contain a complete antigen-recognition and antigen-binding site. This region consists of a dimer of one heavy chain and one light chain variable domain in tight, non-covalent association.

Antibody “light chains” from any vertebrate species can be assigned to one of two clearly distinct types, called kappa and lambda based on amino acid sequences of their constant domains. Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2.

“Single-chain Fv” or “scFv” as used herein, refers to a fusion protein of VH and VL antibody domains, wherein these domains are linked together into a single polypeptide chain. In some embodiments, the Fv polypeptide linker enables the scFv to form the desired structure for antigen binding.

The term “diabodies” refers to small antibody fragments with two antigen-binding sites, which fragments comprise a heavy chain variable domain V_{H} connected to a light chain variable domain V_{L} in the same polypeptide chain. By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993), the contents of each of which are incorporated herein by reference in their entirety.

Antibodies of the present disclosure may be polyclonal or monoclonal or recombinant, produced by methods known in the art or as described in this application. The term “monoclonal antibody” as used herein refers to an antibody obtained from a population of substantially homogeneous cells (or clones), i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen.

The modifier “monoclonal” indicates the character of the antibody as being obtained from a substantially homogeneous population of antibodies, and is not to be construed as requiring production of the antibody by any particular method. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from the hypervariable region from an antibody of the recipient are replaced by residues from the hypervariable region from an antibody of a non-human species (donor antibody) such as mouse, rat, rabbit or nonhuman primate having the desired specificity, affinity, and capacity.

The term “hypervariable region” when used herein in reference to antibodies refers to regions within the antigen binding domain of an antibody comprising the amino acid residues that are responsible for antigen binding. The amino acids present within the hypervariable regions determine the structure of the complementarity determining region (CDR). As used herein, the “CDR” refers to the region of an antibody that comprises a structure that is complimentary to its target antigen or epitope.

In some embodiments, the compositions of the present disclosure may be antibody mimetics. The term “antibody mimetic” refers to any molecule which mimics the function or effect of an antibody and which binds specifically and with high affinity to their molecular targets. As such, antibody mimics include nanobodies and the like.

In some embodiments, antibody mimetics may be those known in the art including, but are not limited to affibody molecules, affilins, affitins, anticalins, avimers, DARPins, Fynomers and Kunitz and domain peptides. In other embodiments, antibody mimetics may include one or more non-peptide region.

As used herein, the term “antibody variant” refers to a biomolecule resembling an antibody in structure and/or function comprising some differences in their amino acid sequence, composition or structure as compared to a native antibody.

The preparation of antibodies, whether monoclonal or polyclonal, is known in the art. Techniques for the production of antibodies are well known in the art and described, e.g. in Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999.

Antibodies of the present disclosure may be characterized by their target molecule(s), by the antigens used to generate them, by their function (whether as agonists or antagonists) and/or by the cell niche in which they function.

Measures of antibody function may be made relative to a standard under normal physiologic conditions, in vitro or in vivo. Measurements may also be made relative to the presence or absence of the antibodies. Such methods of measuring include standard measurement in tissue or fluids such as serum or blood such as Western blot, enzyme-linked immunosorbent assay (ELISA), activity assays, reporter assays, luciferase assays, polymerase chain reaction (PCR) arrays, gene arrays, Real Time reverse transcriptase (RT) PCR and the like.

Antibodies of the present disclosure exert their effects via binding (reversibly or irreversibly) to one or more target sites. While not wishing to be bound by theory, target sites which represent a binding site for an antibody, are most often formed by proteins or protein domains or regions. However, target sites may also include biomolecules such as sugars, lipids, nucleic acid molecules or any other form of binding epitope.

Alternatively, or additionally, antibodies of the present disclosure may function as ligand mimetics or nontraditional payload carriers, acting to deliver or ferry bound or conjugated drug payloads to specific target sites.

Changes elicited by antibodies of the present disclosure may result in a neomorphic change in the cell. As used herein, “a neomorphic change” is a change or alteration that is new or different. Such changes include extracellular, intracellular and cross cellular signaling.

In some embodiments, compounds or agents of the disclosure act to alter or control proteolytic events. Such events may be intracellular or extracellular.

Antibodies of the present disclosure, as well as antigens used to generate them, are primarily amino acid-based molecules. These molecules may be “peptides,” “polypeptides,” or “proteins.”

As used herein, the term “peptide” refers to an amino-acid based molecule having from 2 to 50 or more amino acids. Special designators apply to the smaller peptides with “dipeptide” referring to a two amino acid molecule and “tripeptide” referring to a three amino acid molecule Amino acid based molecules having more than 50 contiguous amino acids are considered polypeptides or proteins.

The terms “amino acid” and “amino acids” refer to all naturally occurring L-alpha-amino acids as well as non-naturally occurring amino acids Amino acids are identified by either the one-letter or three-letter designations as follows: aspartic acid (Asp:D), isoleucine (Ile:I), threonine (Thr:T), leucine (Leu:L), serine (Ser:S), tyrosine (Tyr:Y), glutamic acid (Glu:E), phenylalanine (Phe:F), proline (Pro:P), histidine (His:H), glycine (Gly:G), lysine (Lys:K), alanine (Ala:A), arginine (Arg:R), cysteine (Cys:C), tryptophan (Trp:W), valine (Val:V), glutamine (Gln:Q) methionine (Met:M), asparagines (Asn:N), where the amino acid is listed first followed parenthetically by the three and one letter codes, respectively.

### Hybridizing Oligonucleotides

In some embodiments, oligonucleotides, including those which function via a hybridization mechanism, whether single of double stranded such as antisense molecules, RNAi constructs (including siRNA, saRNA, microRNA, etc.), aptamers and ribozymes may be used to alter or as perturbation stimuli of the gene signaling networks associated with the target gene.

In some embodiments, hybridizing oligonucleotides (e.g., siRNA) may be used to knock down signaling molecules involved in the pathways regulating expression of a target gene such that the expression is altered in the absence of the signaling molecule. For example, once a pathway is identified to negatively regulate the expression of a target gene, a component of the pathway (e.g., a receptor, a protein kinase, a transcription factor) may be knocked down with a RNAi agent (e.g., siRNA) to enhance the expression of the gene. Similarly, once a pathway is identified to positively regulate the expression of a target gene, a component of the pathway (e.g., a receptor, a protein kinase, a transcription factor) may be knocked down with a RNAi agent (e.g., siRNA) to reduce the expression of the gene.

In some embodiments, more than one hybridizing oligonucleotide may be used to target more than one component in the same pathway, or more than one component from different pathways, to alter target gene expression. Such combination therapies may achieve additive or synergetic effects by simultaneously blocking multiple signaling molecules and/or pathways that regulate the expression of a target gene.

As such oligonucleotides may also serve as therapeutics, their therapeutic liabilities and treatment outcomes may be ameliorated or predicted, respectively by interrogating the gene signaling networks of the disclosure.

### Genome Editing Approaches

In certain embodiments, expression of a target gene may be modulated by altering the chromosomal regions defining the insulated neighborhood(s) and/or genome signaling center(s) associated with the target gene.

Methods of altering the gene expression attendant to an insulated neighborhood include altering the signaling center (e.g. using CRISPR/Cas to change the signaling center binding site or repair/replace if mutated). These alterations may result in a variety of results including: activation of cell death pathways prematurely/inappropriately (key to many immune disorders), production of too little/much gene product (also known as the rheostat hypothesis), production of too little/much extracellular secretion of enzymes, prevention of lineage differentiation, switch of lineage pathways, promotion of sternness, initiation or interference with auto regulatory feedback loops, initiation of errors in cell metabolism, inappropriate imprinting/gene silencing, and formation of flawed chromatin states. Additionally, genome editing approaches including those well-known in the art may be used to create new signaling centers by altering the cohesin necklace or moving genes and enhancers.

In certain embodiments, genome editing approaches describe herein may include methods of using site-specific nucleases to introduce single-strand or double-strand DNA breaks at particular locations within the genome. Such breaks can be and regularly are repaired by endogenous cellular processes, such as homology-directed repair (HDR) and non-homologous end joining (NHEJ). HDR is essentially an error-free mechanism that repairs double-strand DNA breaks in the presence of a homologous DNA sequence. The most common form of HDR is homologous recombination. It utilizes a homologous sequence as a template for inserting or replacing a specific DNA sequence at the break point. The template for the homologous DNA sequence can be an endogenous sequence (e.g., a sister chromatid), or an exogenous or supplied sequence (e.g., plasmid or an oligonucleotide). As such, HDR may be utilized to introduce precise alterations such as replacement or insertion at desired regions. In contrast, NHEJ is an error-prone repair mechanism that directly joins the DNA ends resulting from a double-strand break with the possibility of losing, adding or mutating a few nucleotides at the cleavage site. The resulting small deletions or insertions (termed “Indels”) or mutations may disrupt or enhance gene expression. Additionally, if there are two breaks on the same DNA, NHEJ can lead to the deletion or inversion of the intervening segment. Therefore, NHEJ may be utilized to introduce insertions, deletions or mutations at the cleavage site.

### CRISPR/Cas Systems

In certain embodiments, a CRISPR/Cas system may be used to delete CTCF anchor sites to modulate gene expression within the insulated neighborhood associated with that anchor site. See, Hnisz et al., Cell 167, Nov. 17, 2016, which is hereby incorporated by reference in its entirety. Disruption of the boundaries of insulated neighborhood prevents the interactions necessary for proper function of the associated signaling centers. Changes in the expression genes that are immediately adjacent to the deleted neighborhood boundary have also been observed due to such disruptions.

In certain embodiments, a CRISPR/Cas system may be used to modify existing CTCF anchor sites. For example, existing CTCF anchor sites may be mutated or inverted by inducing NHEJ with a CRISPR/Cas nuclease and one or more guide RNAs, or masked by targeted binding with a catalytically inactive CRISPR/Cas enzyme and one or more guide RNAs. Alteration of existing CTCF anchor sites may disrupt the formation of existing insulated neighborhoods and alter the expression of genes located within these insulated neighborhoods.

In certain embodiments, a CRISPR/Cas system may be used to introduce new CTCF anchor sites. CTCF anchor sites may be introduced by inducing HDR at a selected site with a CRISPR/Cas nuclease, one or more guide RNAs and a donor template containing the sequence of a CTCF anchor site. Introduction of new CTCF anchor sites may create new insulated neighborhoods and/or alter existing insulated neighborhoods, which may affect expression of genes that are located adjacent to these insulated neighborhoods.

In certain embodiments, a CRISPR/Cas system may be used to alter signaling centers by changing signaling center binding sites. For example, if a signaling center binding site contains a mutation that affects the assembly of the signaling center with associated transcription factors, the mutated site may be repaired by inducing a double strand DNA break at or near the mutation using a CRISPR/Cas nuclease and one or more guide RNAs in the presence of a supplied corrected donor template.

In certain embodiments, a CRISPR/Cas system may be used to modulate expression of neighborhood genes by binding to a region within an insulated neighborhood (e g , enhancer) and block transcription. Such binding may prevent recruitment of transcription factors to signaling centers and initiation of transcription. The CRISPR/Cas system may be a catalytically inactive CRISPR/Cas system that do not cleave DNA.

In certain embodiments, a CRISPR/Cas system may be used to knockdown expression of neighborhood genes via introduction of short deletions in coding regions of these genes. When repaired, such deletions would result in frame shifts and/or introduce premature stop codons in mRNA produced by the genes followed by the mRNA degradation via nonsense-mediated decay. This may be useful for modulation of expression of activating and repressive components of signaling pathways that would result in decreased or increased expression of genes under control of these pathways including disease genes such as those listed in Table 1.

In other embodiments, a CRISPR/Cas system may also be used to alter cohesion necklace or moving genes and enhancers.

### CRISPR/Cas Enzymes

CRISPR/Cas systems are bacterial adaptive immune systems that utilize RNA-guided endonucleases to target specific sequences and degrade target nucleic acids. They have been adapted for use in various applications in the field of genome editing and/or transcription modulation. Any of the enzymes or orthologs known in the art or disclosed herein may be utilized in the methods herein for genome editing.

In certain embodiments, the CRISPR/Cas system may be a Type II CRISPR/Cas9 system. Cas9 is an endonuclease that functions together with a trans-activating CRISPR RNA (tracrRNA) and a CRISPR RNA (crRNA) to cleave double stranded DNAs. The two RNAs can be engineered to form a single-molecule guide RNA by connecting the 3′ end of the crRNA to the 5′ end of tracrRNA with a linker loop. Jinek et al., Science, 337(6096):816-821 (2012) showed that the CRISPR/Cas9 system is useful for RNA-programmable genome editing, and international patent application WO2013/176772 provides numerous examples and applications of the CRISPR/Cas endonuclease system for site-specific gene editing, which are incorporated herein by reference in their entirety. Exemplary CRISPR/Cas9 systems include those derived from Streptococcus pyogenes, Streptococcus thermophilus, Neisseria meningitidis, Treponema denticola, Streptococcus aureas, and Francisella tularensis.

In certain embodiments, the CRISPR/Cas system may be a Type V CRISPR/Cpf1 system. Cpf1 is a single RNA-guided endonuclease that, in contrast to Type II systems, lacks tracrRNA. Cpf1 produces staggered DNA double-stranded break with a 4 or 5 nucleotide 5′ overhang. Zetsche et al. Cell. 2015 Oct. 22; 163(3):759-71 provides examples of Cpf1 endonuclease that can be used in genome editing applications, which is incorporated herein by reference in its entirety. Exemplary CRISPR/Cpf1 systems include those derived from Francisella tularensis, Acidaminococcus sp., and Lachnospiraceae bacterium.

In certain embodiments, nickase variants of the CRISPR/Cas endonucleases that have one or the other nuclease domain inactivated may be used to increase the specificity of CRISPR-mediated genome editing. Nickases have been shown to promote HDR versus NHEJ. HDR can be directed from individual Cas nickases or using pairs of nickases that flank the target area.

In certain embodiments, catalytically inactive CRISPR/Cas systems may be used to bind to target regions (e.g., CTCF anchor sites or enhancers) and interfere with their function. Cas nucleases such as Cas9 and Cpf1 encompass two nuclease domains. Mutating critical residues at the catalytic sites creates variants that only bind to target sites but do not result in cleavage. Binding to chromosomal regions (e.g., CTCF anchor sites or enhancers) may disrupt proper formation of insulated neighborhoods or signaling centers and therefore lead to altered expression of genes located adjacent to the target region.

In certain embodiments, a CRISPR/Cas system may include additional functional domain(s) fused to the CRISPR/Cas enzyme. The functional domains may be involved in processes including but not limited to transcription activation, transcription repression, DNA methylation, histone modification, and/or chromatin remodeling. Such functional domains include but are not limited to a transcriptional activation domain (e.g., VP64 or KRAB, SID or SID4X), a transcriptional repressor, a recombinase, a transposase, a histone remodeler, a DNA methyltransferase, a cryptochrome, a light inducible/controllable domain or a chemically inducible/controllable domain.

In certain embodiments, a CRISPR/Cas enzyme may be administered to a cell or a patient as one or a combination of the following: one or more polypeptides, one or more mRNAs encoding the polypeptide, or one or more DNAs encoding the polypeptide.

### Guide Nucleic Acid

In certain embodiments, guide nucleic acids may be used to direct the activities of an associated CRISPR/Cas enzymes to a specific target sequence within a target nucleic acid. Guide nucleic acids provide target specificity to the guide nucleic acid and CRISPR/Cas complexes by virtue of their association with the CRISPR/Cas enzymes, and the guide nucleic acids thus can direct the activity of the CRISPR/Cas enzymes.

In one aspect, guide nucleic acids may be RNA molecules. In one aspect, guide RNAs may be single-molecule guide RNAs. In one aspect, guide RNAs may be chemically modified.

In certain embodiments, more than one guide RNAs may be provided to mediate multiple CRISPR/Cas-mediated activities at different sites within the genome.

In certain embodiments, guide RNAs may be administered to a cell or a patient as one or more RNA molecules or one or more DNAs encoding the RNA sequences.

### Ribonucleoprotein Complexes (RNPs)

In one embodiment, the CRISPR/Cas enzyme and guide nucleic acid may each be administered separately to a cell or a patient.

In another embodiment, the CRISPR/Cas enzyme may be pre-complexed with one or more guide nucleic acids. The pre-complexed material may then be administered to a cell or a patient. Such pre-complexed material is known as a ribonucleoprotein particle (RNP).

### Zinc Finger Nucleases

In certain embodiments, genome editing approaches of the present disclosure involve the use of Zinc finger nucleases (ZFNs). Zinc finger nucleases (ZFNs) are modular proteins comprised of an engineered zinc finger DNA binding domain linked to a DNA-cleavage domain. A typical DNA-cleavage domain is the catalytic domain of the type II endonuclease FokI. Because FokI functions only as a dimer, a pair of ZFNs must are required to be engineered to bind to cognate target “half-site” sequences on opposite DNA strands and with precise spacing between them to allow the two enable the catalytically active FokI domains to dimerize. Upon dimerization of the FokI domain, which itself has no sequence specificity per se, a DNA double-strand break is generated between the ZFN half-sites as the initiating step in genome editing.

### Transcription Activator-Like Effector Nucleases (TALENs)

In certain embodiments, genome editing approaches of the present disclosure involve the use of Transcription Activator-Like Effector Nucleases (TALENs). TALENs represent another format of modular nucleases which, similarly to ZFNs, are generated by fusing an engineered DNA binding domain to a nuclease domain, and operate in tandem to achieve targeted DNA cleavage. While the DNA binding domain in ZFN consists of Zinc finger motifs, the TALEN DNA binding domain is derived from transcription activator-like effector (TALE) proteins, which were originally described in the plant bacterial pathogen Xanthomonas sp. TALEs are comprised of tandem arrays of 33-35 amino acid repeats, with each repeat recognizing a single basepair in the target DNA sequence that is typically up to 20 bp in length, giving a total target sequence length of up to 40 bp. Nucleotide specificity of each repeat is determined by the repeat variable diresidue (RVD), which includes just two amino acids at positions 12 and 13. The bases guanine, adenine, cytosine and thymine are predominantly recognized by the four RVDs: Asn-Asn, Asn-Ile, His-Asp and Asn-Gly, respectively. This constitutes a much simpler recognition code than for zinc fingers, and thus represents an advantage over the latter for nuclease design. Nevertheless, as with ZFNs, the protein-DNA interactions of TALENs are not absolute in their specificity, and TALENs have also benefitted from the use of obligate heterodimer variants of the FokI domain to reduce off-target activity.

### IV. FORMULATIONS AND DELIVERY

### Pharmaceutical Compositions

According to the present disclosure the compositions may be prepared as pharmaceutical compositions. It will be understood that such compositions necessarily comprise one or more active ingredients and, most often, a pharmaceutically acceptable excipient.

Relative amounts of the active ingredient, a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the pharmaceutical compositions described herein may comprise at least one payload. As a non-limiting example, the pharmaceutical compositions may contain 1, 2, 3, 4 or 5 payloads.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, rats, birds, including commercially relevant birds such as poultry, chickens, ducks, geese, and/or turkeys.

In some embodiments, compositions are administered to humans, human patients or subjects.

In some embodiments, compositions are administered to mamalian cells. In some embodiments, the cell is a human cell. In some embodiments, the cell is a mouse cell. In some embodiments, the cell is a hepatocyte.

### Formulations

Formulations of the present disclosure can include, without limitation, saline, liposomes, lipid nanoparticles, polymers, peptides, proteins, cells transfected with viral vectors (e.g., for transfer or transplantation into a subject) and combinations thereof.

Formulations of the pharmaceutical compositions described herein may be prepared by any method known or hereafter developed in the art of pharmacology. As used herein the term “pharmaceutical composition” refers to compositions comprising at least one active ingredient and optionally one or more pharmaceutically acceptable excipients.

In general, such preparatory methods include the step of associating the active ingredient with an excipient and/or one or more other accessory ingredients.

Formulations of the compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient.

By way of example, the composition may comprise between 0.1% and 100%, e.g., between 0.5 and 50%, between 1-30%, between 5-80%, at least 80% (w/w) active ingredient.

### Excipients and Diluents

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

### Inactive Ingredients

In some embodiments, the pharmaceutical compositions formulations may comprise at least one inactive ingredient. As used herein, the term “inactive ingredient” refers to one or more agents that do not contribute to the activity of the active ingredient of the pharmaceutical composition included in formulations. In some embodiments, all, none or some of the inactive ingredients which may be used in the formulations of the present disclosure may be approved by the US Food and Drug Administration (FDA).

In one embodiment, the pharmaceutical compositions comprise at least one inactive ingredient such as, but not limited to, 1,2,6-Hexanetriol; 1,2-Dimyristoyl-Sn-Glycero-3-(Phospho-S-(1-Glycerol)); 1,2-Dimyristoyl-Sn-Glycero-3-Phosphocholine; 1,2-Dioleoyl-Sn-Glycero-3-Phosphocholine; 1,2-Dipalmitoyl-Sn-Glycero-3-(Phospho-Rac-(1-Glycerol)); 1,2-Distearoyl-Sn-Glycero-3-(Phospho-Rac-(1-Glycerol)); 1,2-Distearoyl-Sn-Glycero-3-Phosphocholine; 1-O-Tolylbiguanide; 2-Ethyl-1,6-Hexanediol; Acetic Acid; Acetic Acid, Glacial; Acetic Anhydride; Acetone; Acetone Sodium Bisulfite; Acetylated Lanolin Alcohols; Acetylated Monoglycerides; Acetylcysteine; Acetyltryptophan, DL-; Acrylates Copolymer; Acrylic Acid-Isooctyl Acrylate Copolymer; Acrylic Adhesive 788; Activated Charcoal; Adcote 72A103; Adhesive Tape; Adipic Acid; Aerotex Resin 3730; Alanine; Albumin Aggregated; Albumin Colloidal; Albumin Human; Alcohol; Alcohol, Dehydrated; Alcohol, Denatured; Alcohol, Diluted; Alfadex; Alginic Acid; Alkyl Ammonium Sulfonic Acid Betaine; Alkyl Aryl Sodium Sulfonate; Allantoin; Allyl .Alpha.-Ionone; Almond Oil; Alpha-Terpineol; Alpha-Tocopherol; Alpha-Tocopherol Acetate, Dl-; Alpha-Tocopherol, Dl-; Aluminum Acetate; Aluminum Chlorhydroxy Allantoinate; Aluminum Hydroxide; Aluminum Hydroxide—Sucrose, Hydrated; Aluminum Hydroxide Gel; Aluminum Hydroxide Gel F 500; Aluminum Hydroxide Gel F 5000; Aluminum Monostearate; Aluminum Oxide; Aluminum Polyester; Aluminum Silicate; Aluminum Starch Octenylsuccinate; Aluminum Stearate; Aluminum Subacetate; Aluminum Sulfate Anhydrous; Amerchol C; Amerchol-Cab; Aminomethylpropanol; Ammonia; Ammonia Solution; Ammonia Solution, Strong; Ammonium Acetate; Ammonium Hydroxide; Ammonium Lauryl Sulfate; Ammonium Nonoxynol-4 Sulfate; Ammonium Salt Of C-12-C-15 Linear Primary Alcohol Ethoxylate; Ammonium Sulfate; Ammonyx; Amphoteric-2; Amphoteric-9; Anethole; Anhydrous Citric Acid; Anhydrous Dextrose; Anhydrous Lactose; Anhydrous Trisodium Citrate; Aniseed Oil; Anoxid Sbn; Antifoam; Antipyrine; Apaflurane; Apricot Kernel Oil Peg-6 Esters; Aquaphor; Arginine; Arlacel; Ascorbic Acid; Ascorbyl Palmitate; Aspartic Acid; Balsam Peru; Barium Sulfate; Beeswax; Beeswax, Synthetic; Beheneth-10; Bentonite; Benzalkonium Chloride; Benzenesulfonic Acid; Benzethonium Chloride; Benzododecinium Bromide; Benzoic Acid; Benzyl Alcohol; Benzyl Benzoate; Benzyl Chloride; Betadex; Bibapcitide; Bismuth Subgallate; Boric Acid; Brocrinat; Butane; Butyl Alcohol; Butyl Ester Of Vinyl Methyl Ether/Maleic Anhydride Copolymer (125000 Mw); Butyl Stearate; Butylated Hydroxyanisole; Butylated Hydroxytoluene; Butylene Glycol; Butylparaben; Butyric Acid; C20-40 Pareth-24; Caffeine; Calcium; Calcium Carbonate; Calcium Chloride; Calcium Gluceptate; Calcium Hydroxide; Calcium Lactate; Calcobutrol; Caldiamide Sodium; Caloxetate Trisodium; Calteridol Calcium; Canada Balsam; Caprylic/Capric Triglyceride; Caprylic/Capric/Stearic Triglyceride; Captan; Captisol; Caramel; Carbomer 1342; Carbomer 1382; Carbomer 934; Carbomer 934p; Carbomer 940; Carbomer 941; Carbomer 980; Carbomer 981; Carbomer Homopolymer Type B (Allyl Pentaerythritol Crosslinked); Carbomer Homopolymer Type C (Allyl Pentaerythritol Crosslinked); Carbon Dioxide; Carboxy Vinyl Copolymer; Carboxymethylcellulose; Carboxymethylcellulose Sodium; Carboxypolymethylene; Carrageenan; Carrageenan Salt; Castor Oil; Cedar Leaf Oil; Cellulose; Cellulose, Microcrystalline; Cerasynt-Se; Ceresin; Ceteareth-12; Ceteareth-15; Ceteareth-30; Cetearyl Alcohol/Ceteareth-20; Cetearyl Ethylhexanoate; Ceteth-10; Ceteth-2; Ceteth-20; Ceteth-23; Cetostearyl Alcohol; Cetrimonium Chloride; Cetyl Alcohol; Cetyl Esters Wax; Cetyl Palmitate; Cetylpyridinium Chloride; Chlorobutanol; Chlorobutanol Hemihydrate; Chlorobutanol, Anhydrous; Chlorocresol; Chloroxylenol; Cholesterol; Choleth; Choleth-24; Citrate; Citric Acid; Citric Acid Monohydrate; Citric Acid, Hydrous; Cocamide Ether Sulfate; Cocamine Oxide; Coco Betaine; Coco Diethanolamide; Coco Monoethanolamide; Cocoa Butter; Coco-Glycerides; Coconut Oil; Coconut Oil, Hydrogenated; Coconut Oil/Palm Kernel Oil Glycerides, Hydrogenated; Cocoyl Caprylocaprate; Cola Nitida Seed Extract; Collagen; Coloring Suspension; Corn Oil; Cottonseed Oil; Cream Base; Creatine; Creatinine; Cresol; Croscarmellose Sodium; Crospovidone; Cupric Sulfate; Cupric Sulfate Anhydrous; Cyclomethicone; Cyclomethicone/Dimethicone Copolyol; Cysteine; Cysteine Hydrochloride; Cysteine Hydrochloride Anhydrous; Cysteine, Dl-; D&C Red No. 28; D&C Red No. 33; D&C Red No. 36; D&C Red No. 39; D&C Yellow No. 10; Dalfampridine; Daubert 1-5 Pestr (Matte) 164z; Decyl Methyl Sulfoxide; Dehydag Wax Sx; Dehydroacetic Acid; Dehymuls E; Denatonium Benzoate; Deoxycholic Acid; Dextran; Dextran 40; Dextrin; Dextrose; Dextrose Monohydrate; Dextrose Solution; Diatrizoic Acid; Diazolidinyl Urea; Dichlorobenzyl Alcohol; Dichlorodifluoromethane; Dichlorotetrafluoroethane; Diethanolamine; Diethyl Pyrocarbonate; Diethyl Sebacate; Diethylene Glycol Monoethyl Ether; Diethylhexyl Phthalate; Dihydroxyaluminum Aminoacetate; Diisopropanolamine; Diisopropyl Adipate; Diisopropyl Dilinoleate; Dimethicone 350; Dimethicone Copolyol; Dimethicone Mdx4-4210; Dimethicone Medical Fluid 360; Dimethyl Isosorbide; Dimethyl Sulfoxide; Dimethylaminoethyl Methacrylate-Butyl Methacrylate—Methyl Methacrylate Copolymer; Dimethyldioctadecylammonium Bentonite; Dimethylsiloxane/Methylvinylsiloxane Copolymer; Dinoseb Ammonium Salt; Dipalmitoylphosphatidylglycerol, Dl-; Dipropylene Glycol; Disodium Cocoamphodiacetate; Disodium Laureth Sulfosuccinate; Disodium Lauryl Sulfosuccinate; Disodium Sulfosalicylate; Disofenin; Divinylbenzene Styrene Copolymer; Dmdm Hydantoin; Docosanol; Docusate Sodium; Duro-Tak 280-2516; Duro-Tak 387-2516; Duro-Tak 80-1196; Duro-Tak 87-2070; Duro-Tak 87-2194; Duro-Tak 87-2287; Duro-Tak 87-2296; Duro-Tak 87-2888; Duro-Tak 87-2979; Edetate Calcium Disodium; Edetate Disodium; Edetate Disodium Anhydrous; Edetate Sodium; Edetic Acid; Egg Phospholipids; Entsufon; Entsufon Sodium; Epilactose; Epitetracycline Hydrochloride; Essence Bouquet 9200; Ethanolamine Hydrochloride; Ethyl Acetate; Ethyl Oleate; Ethylcelluloses; Ethylene Glycol; Ethylene Vinyl Acetate Copolymer; Ethylenediamine; Ethylenediamine Dihydrochloride; Ethylene-Propylene Copolymer; Ethylene-Vinyl Acetate Copolymer (28% Vinyl Acetate); Ethylene-Vinyl Acetate Copolymer (9% Vinylacetate); Ethylhexyl Hydroxystearate; Ethylparaben; Eucalyptol; Exametazime; Fat, Edible; Fat, Hard; Fatty Acid Esters; Fatty Acid Pentaerythriol Ester; Fatty Acids; Fatty Alcohol Citrate; Fatty Alcohols; Fd&C Blue No. 1; Fd&C Green No. 3; Fd&C Red No. 4; Fd&C Red No. 40; Fd&C Yellow No. 10 (Delisted); Fd&C Yellow No. 5; Fd&C Yellow No. 6; Ferric Chloride; Ferric Oxide; Flavor 89-186; Flavor 89-259; Flavor Df-119; Flavor Df-1530; Flavor Enhancer; Flavor Fig 827118; Flavor Raspberry Pfc-8407; Flavor Rhodia Pharmaceutical No. Rf 451; Fluorochlorohydrocarbons; Formaldehyde; Formaldehyde Solution; Fractionated Coconut Oil; Fragrance 3949-5; Fragrance 520a; Fragrance 6.007; Fragrance 91-122; Fragrance 9128-Y; Fragrance 93498g; Fragrance Balsam Pine No. 5124; Fragrance Bouquet 10328; Fragrance Chemoderm 6401-B; Fragrance Chemoderm 6411; Fragrance Cream No. 73457; Fragrance Cs-28197; Fragrance Felton 066m; Fragrance Firmenich 47373; Fragrance Givaudan Ess 9090/1c; Fragrance H-6540; Fragrance Herbal 10396; Fragrance Nj-1085; Fragrance P O Fl-147; Fragrance Pa 52805; Fragrance Pera Derm D; Fragrance Rbd-9819; Fragrance Shaw Mudge U-7776; Fragrance Tf 044078; Fragrance Ungerer Honeysuckle K 2771; Fragrance Ungerer N5195; Fructose; Gadolinium Oxide; Galactose; Gamma Cyclodextrin; Gelatin; Gelatin, Crosslinked; Gelfoam Sponge; Gellan Gum (Low Acyl); Gelva 737; Gentisic Acid; Gentisic Acid Ethanolamide; Gluceptate Sodium; Gluceptate Sodium Dihydrate; Gluconolactone; Glucuronic Acid; Glutamic Acid, Dl-; Glutathione; Glycerin; Glycerol Ester Of Hydrogenated Rosin; Glyceryl Citrate; Glyceryl Isostearate; Glyceryl Laurate; Glyceryl Monostearate; Glyceryl Oleate; Glyceryl Oleate/Propylene Glycol; Glyceryl Palmitate; Glyceryl Ricinoleate; Glyceryl Stearate; Glyceryl Stearate—Laureth-23; Glyceryl Stearate/Peg Stearate; Glyceryl Stearate/Peg-100 Stearate; Glyceryl Stearate/Peg-40 Stearate; Glyceryl Stearate-Stearamidoethyl Diethylamine; Glyceryl Trioleate; Glycine; Glycine Hydrochloride; Glycol Distearate; Glycol Stearate; Guanidine Hydrochloride; Guar Gum; Hair Conditioner (18n195-1m); Heptane; Hetastarch; Hexylene Glycol; High Density Polyethylene; Histidine; Human Albumin Microspheres; Hyaluronate Sodium; Hydrocarbon; Hydrocarbon Gel, Plasticized; Hydrochloric Acid; Hydrochloric Acid, Diluted; Hydrocortisone; Hydrogel Polymer; Hydrogen Peroxide; Hydrogenated Castor Oil; Hydrogenated Palm Oil; Hydrogenated Palm/Palm Kernel Oil Peg-6 Esters; Hydrogenated Polybutene 635-690; Hydroxide Ion; Hydroxyethyl Cellulose; Hydroxyethylpiperazine Ethane Sulfonic Acid; Hydroxymethyl Cellulose; Hydroxyoctacosanyl Hydroxystearate; Hydroxypropyl Cellulose; Hydroxypropyl Methylcellulose 2906; Hydroxypropyl-Beta-cyclodextrin; Hypromellose 2208 (15000 Mpa·S); Hypromellose 2910 (15000 Mpa·S); Hypromelloses; Imidurea; Iodine; Iodoxamic Acid; Iofetamine Hydrochloride; Irish Moss Extract; Isobutane; Isoceteth-20; Isoleucine; Isooctyl Acrylate; Isopropyl Alcohol; Isopropyl Isostearate; Isopropyl Myristate; Isopropyl Myristate—Myristyl Alcohol; Isopropyl Palmitate; Isopropyl Stearate; Isostearic Acid; Isostearyl Alcohol; Isotonic Sodium Chloride Solution; Jelene; Kaolin; Kathon Cg; Kathon Cg II; Lactate; Lactic Acid; Lactic Acid, Dl-; Lactic Acid, L-; Lactobionic Acid; Lactose; Lactose Monohydrate; Lactose, Hydrous; Laneth; Lanolin; Lanolin Alcohol—Mineral Oil; Lanolin Alcohols; Lanolin Anhydrous; Lanolin Cholesterols; Lanolin Nonionic Derivatives; Lanolin, Ethoxylated; Lanolin, Hydrogenated; Lauralkonium Chloride; Lauramine Oxide; Laurdimonium Hydrolyzed Animal Collagen; Laureth Sulfate; Laureth-2; Laureth-23; Laureth-4; Lauric Diethanolamide; Lauric Myristic Diethanolamide; Lauroyl Sarcosine; Lauryl Lactate; Lauryl Sulfate; Lavandula Angustifolia Flowering Top; Lecithin; Lecithin Unbleached; Lecithin, Egg; Lecithin, Hydrogenated; Lecithin, Hydrogenated Soy; Lecithin, Soybean; Lemon Oil; Leucine; Levulinic Acid; Lidofenin; Light Mineral Oil; Light Mineral Oil (85 Ssu); Limonene, (+/−)-; Lipocol Sc-15; Lysine; Lysine Acetate; Lysine Monohydrate; Magnesium Aluminum Silicate; Magnesium Aluminum Silicate Hydrate; Magnesium Chloride; Magnesium Nitrate; Magnesium Stearate; Maleic Acid; Mannitol; Maprofix; Mebrofenin; Medical Adhesive Modified S-15; Medical Antiform A-F Emulsion; Medronate Disodium; Medronic Acid; Meglumine; Menthol; Metacresol; Metaphosphoric Acid; Methanesulfonic Acid; Methionine; Methyl Alcohol; Methyl Gluceth-10; Methyl Gluceth-20; Methyl Gluceth-20 Sesquistearate; Methyl Glucose Sesquistearate; Methyl Laurate; Methyl Pyrrolidone; Methyl Salicylate; Methyl Stearate; Methylboronic Acid; Methylcellulose (4000 Mpa·S); Methylcelluloses; Methylchloroisothiazolinone; Methylene Blue; Methylisothiazolinone; Methylparaben; Microcrystalline Wax; Mineral Oil; Mono And Diglyceride; Monostearyl Citrate; Monothioglycerol; Multisterol Extract; Myristyl Alcohol; Myristyl Lactate; Myristyl-.Gamma.-Picolinium Chloride; N-(Carbamoyl-Methoxy Peg-40)-1,2-Distearoyl-Cephalin Sodium; N,N-Dimethylacetamide; Niacinamide; Nioxime; Nitric Acid; Nitrogen; Nonoxynol Iodine; Nonoxynol-15; Nonoxynol-9; Norflurane; Oatmeal; Octadecene-1/Maleic Acid Copolymer; Octanoic Acid; Octisalate; Octoxynol-1; Octoxynol-40; Octoxynol-9; Octyldodecanol; Octylphenol Polymethylene; Oleic Acid; Oleth-10/Oleth-5; Oleth-2; Oleth-20; Oleyl Alcohol; Oleyl Oleate; Olive Oil; Oxidronate Disodium; Oxyquinoline; Palm Kernel Oil; Palmitamine Oxide; Parabens; Paraffin; Paraffin, White Soft; Parfum Creme 45/3; Peanut Oil; Peanut Oil, Refined; Pectin; Peg 6-32 Stearate/Glycol Stearate; Peg Vegetable Oil; Peg-100 Stearate; Peg-12 Glyceryl Laurate; Peg-120 Glyceryl Stearate; Peg-120 Methyl Glucose Dioleate; Peg-15 Cocamine; Peg-150 Distearate; Peg-2 Stearate; Peg-20 Sorbitan Isostearate; Peg-22 Methyl Ether/Dodecyl Glycol Copolymer; Peg-25 Propylene Glycol Stearate; Peg-4 Dilaurate; Peg-4 Laurate; Peg-40 Castor Oil; Peg-40 Sorbitan Diisostearate; Peg-45/Dodecyl Glycol Copolymer; Peg-5 Oleate; Peg-50 Stearate; Peg-54 Hydrogenated Castor Oil; Peg-6 Isostearate; Peg-60 Castor Oil; Peg-60 Hydrogenated Castor Oil; Peg-7 Methyl Ether; Peg-75 Lanolin; Peg-8 Laurate; Peg-8 Stearate; Pegoxol 7 Stearate; Pentadecalactone; Pentaerythritol Cocoate; Pentasodium Pentetate; Pentetate Calcium Trisodium; Pentetic Acid; Peppermint Oil; Perflutren; Perfume 25677; Perfume Bouquet; Perfume E-1991; Perfume Gd 5604; Perfume Tana 90/42 Scba; Perfume W-1952-1; Petrolatum; Petrolatum, White; Petroleum Distillates; Phenol; Phenol, Liquefied; Phenonip; Phenoxyethanol; Phenylalanine; Phenylethyl Alcohol; Phenylmercuric Acetate; Phenylmercuric Nitrate; Phosphatidyl Glycerol, Egg; Phospholipid; Phospholipid, Egg; Phospholipon 90g; Phosphoric Acid; Pine Needle Oil (Pinus Sylvestris); Piperazine Hexahydrate; Plastibase-50w; Polacrilin; Polidronium Chloride; Poloxamer 124; Poloxamer 181; Poloxamer 182; Poloxamer 188; Poloxamer 237; Poloxamer 407; Poly(Bis(P-Carboxyphenoxy)Propane Anhydride): Sebacic Acid; Poly(Dimethylsiloxane/Methylvinylsiloxane/Methylhydrogensiloxane) Dimethylvinyl Or Dimethylhydroxy Or Trimethyl Endblocked; Poly(Dl-Lactic-Co-Glycolic Acid), (50:50; Poly(Dl-Lactic-Co-Glycolic Acid), Ethyl Ester Terminated, (50:50; Polyacrylic Acid (250000 Mw); Polybutene (1400 Mw); Polycarbophil; Polyester; Polyester Polyamine Copolymer; Polyester Rayon; Polyethylene Glycol 1000; Polyethylene Glycol 1450; Polyethylene Glycol 1500; Polyethylene Glycol 1540; Polyethylene Glycol 200; Polyethylene Glycol 300; Polyethylene Glycol 300-1600; Polyethylene Glycol 3350; Polyethylene Glycol 400; Polyethylene Glycol 4000; Polyethylene Glycol 540; Polyethylene Glycol 600; Polyethylene Glycol 6000; Polyethylene Glycol 8000; Polyethylene Glycol 900; Polyethylene High Density Containing Ferric Oxide Black (<1%); Polyethylene Low Density Containing Barium Sulfate (20-24%); Polyethylene T; Polyethylene Terephthalates; Polyglactin; Polyglyceryl-3 Oleate; Polyglyceryl-4 Oleate; Polyhydroxyethyl Methacrylate; Polyisobutylene; Polyisobutylene (1100000 Mw); Polyisobutylene (35000 Mw); Polyisobutylene 178-236; Polyisobutylene 241-294; Polyisobutylene 35-39; Polyisobutylene Low Molecular Weight; Polyisobutylene Medium Molecular Weight; Polyisobutylene/Polybutene Adhesive; Polylactide; Polyols; Polyoxyethylene—Polyoxypropylene 1800; Polyoxyethylene Alcohols; Polyoxyethylene Fatty Acid Esters; Polyoxyethylene Propylene; Polyoxyl 20 Cetostearyl Ether; Polyoxyl 35 Castor Oil; Polyoxyl 40 Hydrogenated Castor Oil; Polyoxyl 40 Stearate; Polyoxyl 400 Stearate; Polyoxyl 6 And Polyoxyl 32 Palmitostearate; Polyoxyl Distearate; Polyoxyl Glyceryl Stearate; Polyoxyl Lanolin; Polyoxyl Palmitate; Polyoxyl Stearate; Polypropylene; Polypropylene Glycol; Polyquaternium-10; Polyquaternium-7 (70/30 Acrylamide/Dadmac; Polysiloxane; Polysorbate 20; Polysorbate 40; Polysorbate 60; Polysorbate 65; Polysorbate 80; Polyurethane; Polyvinyl Acetate; Polyvinyl Alcohol; Polyvinyl Chloride; Polyvinyl Chloride-Polyvinyl Acetate Copolymer; Polyvinylpyridine; Poppy Seed Oil; Potash; Potassium Acetate; Potassium Alum; Potassium Bicarbonate; Potassium Bisulfite; Potassium Chloride; Potassium Citrate; Potassium Hydroxide; Potassium Metabisulfite; Potassium Phosphate, Dibasic; Potassium Phosphate, Monobasic; Potassium Soap; Potassium Sorbate; Povidone Acrylate Copolymer; Povidone Hydrogel; Povidone K17; Povidone K25; Povidone K29/32; Povidone K30; Povidone K90; Povidone K90f; Povidone/Eicosene Copolymer; Povidones; Ppg-12/Smdi Copolymer; Ppg-15 Stearyl Ether; Ppg-20 Methyl Glucose Ether Distearate; Ppg-26 Oleate; Product Wat; Proline; Promulgen D; Promulgen G; Propane; Propellant A-46; Propyl Gallate; Propylene Carbonate; Propylene Glycol; Propylene Glycol Diacetate; Propylene Glycol Dicaprylate; Propylene Glycol Monolaurate; Propylene Glycol Monopalmitostearate; Propylene Glycol Palmitostearate; Propylene Glycol Ricinoleate; Propylene Glycol/Diazolidinyl Urea/Methylparaben/Propylparben; Propylparaben; Protamine Sulfate; Protein Hydrolysate; Pvm/Ma Copolymer; Quaternium-15; Quaternium-15 Cis-Form; Quaternium-52; Ra-2397; Ra-3011; Saccharin; Saccharin Sodium; Saccharin Sodium Anhydrous; Safflower Oil; Sd Alcohol 3a; Sd Alcohol 40; Sd Alcohol 40-2; Sd Alcohol 40b; Sepineo P 600; Serine; Sesame Oil; Shea Butter; Silastic Brand Medical Grade Tubing; Silastic Medical Adhesive,Silicone Type A; Silica, Dental; Silicon; Silicon Dioxide; Silicon Dioxide, Colloidal; Silicone; Silicone Adhesive 4102; Silicone Adhesive 4502; Silicone Adhesive Bio-Psa Q7-4201; Silicone Adhesive Bio-Psa Q7-4301; Silicone Emulsion; Silicone/Polyester Film Strip; Simethicone; Simethicone Emulsion; Sipon Ls 20np; Soda Ash; Sodium Acetate; Sodium Acetate Anhydrous; Sodium Alkyl Sulfate; Sodium Ascorbate; Sodium Benzoate; Sodium Bicarbonate; Sodium Bisulfate; Sodium Bisulfite; Sodium Borate; Sodium Borate Decahydrate; Sodium Carbonate; Sodium Carbonate Decahydrate; Sodium Carbonate Monohydrate; Sodium Cetostearyl Sulfate; Sodium Chlorate; Sodium Chloride; Sodium Chloride Injection; Sodium Chloride Injection, Bacteriostatic; Sodium Cholesteryl Sulfate; Sodium Citrate; Sodium Cocoyl Sarcosinate; Sodium Desoxycholate; Sodium Dithionite; Sodium Dodecylbenzenesulfonate; Sodium Formaldehyde Sulfoxylate; Sodium Gluconate; Sodium Hydroxide; Sodium Hypochlorite; Sodium Iodide; Sodium Lactate; Sodium Lactate, L-; Sodium Laureth-2 Sulfate; Sodium Laureth-3 Sulfate; Sodium Laureth-5 Sulfate; Sodium Lauroyl Sarcosinate; Sodium Lauryl Sulfate; Sodium Lauryl Sulfoacetate; Sodium Metabisulfite; Sodium Nitrate; Sodium Phosphate; Sodium Phosphate Dihydrate; Sodium Phosphate, Dibasic; Sodium Phosphate, Dibasic, Anhydrous; Sodium Phosphate, Dibasic, Dihydrate; Sodium Phosphate, Dibasic, Dodecahydrate; Sodium Phosphate, Dibasic, Heptahydrate; Sodium Phosphate, Monobasic; Sodium Phosphate, Monobasic, Anhydrous; Sodium Phosphate, Monobasic, Dihydrate; Sodium Phosphate, Monobasic, Monohydrate; Sodium Polyacrylate (2500000 Mw); Sodium Pyrophosphate; Sodium Pyrrolidone Carboxylate; Sodium Starch Glycolate; Sodium Succinate Hexahydrate; Sodium Sulfate; Sodium Sulfate Anhydrous; Sodium Sulfate Decahydrate; Sodium Sulfite; Sodium Sulfosuccinated Undecyclenic Monoalkylolamide; Sodium Tartrate; Sodium Thioglycolate; Sodium Thiomalate; Sodium Thiosulfate; Sodium Thiosulfate Anhydrous; Sodium Trimetaphosphate; Sodium Xylenesulfonate; Somay 44; Sorbic Acid; Sorbitan; Sorbitan Isostearate; Sorbitan Monolaurate; Sorbitan Monooleate; Sorbitan Monopalmitate; Sorbitan Monostearate; Sorbitan Sesquioleate; Sorbitan Trioleate; Sorbitan Tristearate; Sorbitol; Sorbitol Solution; Soybean Flour; Soybean Oil; Spearmint Oil; Spermaceti; Squalane; Stabilized Oxychloro Complex; Stannous 2-Ethylhexanoate; Stannous Chloride; Stannous Chloride Anhydrous; Stannous Fluoride; Stannous Tartrate; Starch; Starch 1500, Pregelatinized; Starch, Corn; Stearalkonium Chloride; Stearalkonium Hectorite/Propylene Carbonate; Stearamidoethyl Diethylamine; Steareth-10; Steareth-100; Steareth-2; Steareth-20; Steareth-21; Steareth-40; Stearic Acid; Stearic Diethanolamide; Stearoxytrimethylsilane; Steartrimonium Hydrolyzed Animal Collagen; Stearyl Alcohol; Sterile Water For Inhalation; Styrene/Isoprene/Styrene Block Copolymer; Succimer; Succinic Acid; Sucralose; Sucrose; Sucrose Distearate; Sucrose Polyesters; Sulfacetamide Sodium; Sulfobutylether .Beta.-Cyclodextrin; Sulfur Dioxide; Sulfuric Acid; Sulfurous Acid; Surfactol Qs; Tagatose, D-; Talc; Tall Oil; Tallow Glycerides; Tartaric Acid; Tartaric Acid, Dl-; Tenox; Tenox-2; Tert-Butyl Alcohol; Tert-Butyl Hydroperoxide; Tert-Butylhydroquinone; Tetrakis(2-Methoxyisobutylisocyanide)Copper(I) Tetrafluoroborate; Tetrapropyl Orthosilicate; Tetrofosmin; Theophylline; Thimerosal; Threonine; Thymol; Tin; Titanium Dioxide; Tocopherol; Tocophersolan; Total parenteral nutrition, lipid emulsion; Triacetin; Tricaprylin; Trichloromonofluoromethane; Trideceth-10; Triethanolamine Lauryl Sulfate; Trifluoroacetic Acid; Triglycerides, Medium Chain; Trihydroxystearin; Trilaneth-4 Phosphate; Trilaureth-4 Phosphate; Trisodium Citrate Dihydrate; Trisodium Hedta; Triton 720; Triton X-200; Trolamine; Tromantadine; Tromethamine (TRIS); Tryptophan; Tyloxapol; Tyrosine; Undecylenic Acid; Union 76 Amsco-Res 6038; Urea; Valine; Vegetable Oil; Vegetable Oil Glyceride, Hydrogenated; Vegetable Oil, Hydrogenated; Versetamide; Viscarin; Viscose/Cotton; Vitamin E; Wax, Emulsifying; Wecobee Fs; White Ceresin Wax; White Wax; Xanthan Gum; Zinc; Zinc Acetate; Zinc Carbonate; Zinc Chloride; and Zinc Oxide.

Pharmaceutical composition formulations disclosed herein may include cations or anions. In one embodiment, the formulations include metal cations such as, but not limited to, Zn2+, Ca2+, Cu2+, Mn2+, Mg+ and combinations thereof. As a non-limiting example, formulations may include polymers and complexes with a metal cation (See e.g., U.S. Pat. Nos. 6,265,389 and 6,555,525, each of which is herein incorporated by reference in its entirety).

Formulations may also include one or more pharmaceutically acceptable salts. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids.

Solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

### V. ADMINISTRATION AND DOSING

### Administration

The terms “administering” and “introducing” are used interchangeable herein and refer to the delivery of the pharmaceutical composition into a cell or a subject. In the case of delivery to a subject, the pharmaceutical composition is delivered by a method or route that results in at least partial localization of the introduced cells at a desired site, such as hepatocytes, such that a desired effect(s) is produced.

In one aspect of the method, the pharmaceutical composition may be administered via a route such as, but not limited to, enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis and spinal.

Modes of administration include injection, infusion, instillation, and/or ingestion. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some examples, the route is intravenous. For the delivery of cells, administration by injection or infusion can be made.

The cells can be administered systemically. The phrases “systemic administration,” “administered systemically”, “peripheral administration” and “administered peripherally” refer to the administration other than directly into a target site, tissue, or organ, such that it enters, instead, the subject's circulatory system and, thus, is subject to metabolism and other like processes.

### Dosing

The term “effective amount” refers to the amount of the active ingredient needed to prevent or alleviate at least one or more signs or symptoms of a specific disease and/or condition, and relates to a sufficient amount of a composition to provide the desired effect. The term “therapeutically effective amount” therefore refers to an amount of active ingredient or a composition comprising the active ingredient that is sufficient to promote a particular effect when administered to a typical subject. An effective amount would also include an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate “effective amount” can be determined by one of ordinary skill in the art using routine experimentation.

The pharmaceutical, diagnostic, or prophylactic compositions may be administered to a subject using any amount and any route of administration effective for preventing, treating, managing, or diagnosing diseases, disorders and/or conditions. The exact amount required will vary from subject to subject, depending on the species, age, and general condition of the subject, the severity of the disease, the particular composition, its mode of administration, its mode of activity, and the like. The subject may be a human, a mammal, or an animal. Compositions are typically formulated in unit dosage form for ease of administration and uniformity of dosage. It will be understood, however, that the total daily usage of the may be decided by the attending physician within the scope of sound medical judgment. The specific therapeutically effective, prophylactically effective, or appropriate diagnostic dose level for any particular individual will depend upon a variety of factors including the disorder being treated and the severity of the disorder; the activity of the specific payload employed; the specific composition employed; the age, body weight, general health, sex and diet of the patient; the time of administration, and route of administration; the duration of the treatment; drugs used in combination or coincidental with the active ingredient; and like factors well known in the medical arts.

In certain embodiments, pharmaceutical compositions may be administered at dosage levels sufficient to deliver from about 0.01 mg/kg to about 100 mg/kg, from about 0.01 mg/kg to about 0.05 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect.

The desired dosage of the composition may be delivered only once, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of “single unit dose” or total daily dose into two or more doses, e.g., two or more administrations of the “single unit dose”. As used herein, a “single unit dose” is a dose of any therapeutic administered in one dose/at one time/single route/single point of contact, i.e., single administration event.

Described herein are compositions and methods for perturbation of genomic signaling centers (GSCs) or entire gene signaling networks (GSNs) for the treatment of a genetic disease, such as fibronectin glomerulopathy, hereditary coproporphyria and others. The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

The present disclosure is further illustrated by the following non-limiting examples.

### VII. EXAMPLES

### Example 1. Experimental Procedures

A. Human Hepatocyte Cell Culture

Human hepatocytes were obtained from two donors from Massachusetts General Hospital, namely MGH54 and MGH63, and one donor from Lonza, namely HUM4111B. Cryopreserved hepatocytes were cultured in plating media for 16 hours, transferred to maintenance media for 4 hours. Cultured on serum-free media for 2 hours, then a compound was added. The hepatocytes were maintained on the serum-free media for 16 hours prior to gene expression analysis. Primary human hepatocytes were stored in the vapor phase of a liquid nitrogen freezer (about −130° C.).

To seed the primary human hepatocytes, vials of cells were retrieved from the LN₂ freezer, thawed in a 37° C. water bath, and swirled gently until only a sliver of ice remains. Using a 10 ml serological pipet, cells were gently pipetted out of the vial and gently pipetted down the side of 50 mL conical tube containing 20 mL cold thaw medium. The vial was rinsed with about 1 mL of thaw medium, and the rinse was added to the conical tube. Up to 2 vials may be added to one tube of 20 mL thaw medium.

The conical tube(s) were gently inverted 2-3 times and centrifuged at 100 g for 10 minutes at 4° C. with reduced braking (e.g. 4 out of 9). The thaw medium slowly was slowly aspirated to avoid the pellet. 4 mL cold plating medium was added slowly down the side (8 mL if combined 2 vials to 1 tube), and the vial was inverted gently several times to resuspend cells.

Cells were kept on ice until 100 μl of well-mixed cells were added to 400 μl diluted Trypan blue and mixed by gentle inversion. They were counted using a hemocytometer (or Cellometer), and viability and viable cells/mL were noted. Cells were diluted to a desired concentration and seeded on collagen I-coated plates. Cells were pipetted slowly and gently onto plate, only 1-2 wells at a time. The remaining cells were mixed in the tubes frequently by gentle inversion. Cells were seeded at about 8.5×10⁶ cells per plate in 6 mL cold plating medium (10cm). Alternatively, cells were seeded at about 1.5×10⁶ per well for a 6-well plate (1 mL medium/well); 7×10⁵ per well for 12-well plate (0.5 mL/well); or 3.75×10⁵ per well for a 24-well plate (0.5 mL/well)

After all cells and medium were added to the plate, the plate was transferred to an incubator (37° C., 5% CO₂, about 90% humidity) and rocked forwards and backwards, then side to side several times each to distribute cells evenly across the plate or wells. The plate(s) were rocked again every 15 minutes for the first hour post-plating. About 4 hours post-plating (or first thing the morning if cells were plated in the evening), cells were washed once with PBS and complete maintenance medium was added. The primary human hepatocytes were maintained in the maintenance medium and transferred to fresh medium daily.

B. Starvation and Compound Treatment of Human Hepatocytes

Human hepatocytes cultured as described above were plated in 24-well format, adding 375,000 cells per well in a volume of 500 ul plating medium. Four hours before treatment, cells were washed with PBS and the medium was changed to either: fresh maintenance medium (complete) or modified maintenance medium.

Compound stocks were prepared at 1000× final concentration and added in a 2-step dilution to the medium to reduce risk of a compound precipitating out of solution when added to the cells, and to ensure reasonable pipetting volumes. One at a time, each compound was first diluted 10-fold in warm (about 37° C.) modified maintenance medium (initial dilution=ID), mixed by vortexing, and the ID was diluted 100-fold into the cell culture (e.g. 5.1 ul into 1 well of a 24-well plate containing 0.5 mL medium). The plate was mixed by carefully swirling and after all wells were treated and returned to the incubator overnight. If desired, separate plates/wells were treated with vehicle-only controls and/or positive controls. If using multi-well plates, controls were included on each plate. After about 18 hours, cells were harvested for further analysis, e.g., ChIP-seq, RNA-seq, ATAC-seq, etc.

C. Mouse Hepatocyte Cell Culture and Compound Treatment

Female C57BL/6 mouse hepatocytes (F005152-cryopreserved) were purchased from BioreclamationIVT as a pool of 45 donors. Cells were plated in InvitroGRO CP Rodent Medium (Z990028) and Torpedo Rodent Antibiotic Mix (Z99027) on Collagen-coated 24-well plates for 24 hours at 200K cells/well in 0.5 mL media. Compound stocks in 10 mM DMSO, were diluted to 10 uM (with final concentration of 1% DMSO), and applied on cells in biological triplicates. Medium was removed after 20 hours and cells processed for further analysis, e.g. qRT-PCR.

D. Media Composition

The thaw medium contained 6 mL isotonic percoll and 14 mL high glucose DMEM (Invitrogen #11965 or similar). The plating medium contained 100 mL Williams E medium (Invitrogen #A1217601, without phenol red) and the supplement pack #CM3000 from ThermoFisher Plating medium containing 5 mL FBS, 10 μl dexamethasone, and 3.6 mL plating/maintenance cocktail. Stock trypan blue (0.4%, Invitrogen #15250) was diluted 1:5 in PBS. Normocin was added at 1:500 to both the thaw medium and the plating medium.

The ThermoFisher complete maintenance medium contained supplement pack #CM4000 (1 μl dexamethasone and 4 mL maintenance cocktail) and 100 mL Williams E (Invitrogen #A1217601, without phenol red).

The modified maintenance media had no stimulating factors (dexamethasone, insulin, etc.), and contained100 mL Williams E (Invitrogen #A1217601, without phenol red), 1mL L-Glutamine (Sigma #G7513) to 2 mM, 1.5 mL HEPES (VWR #J848) to 15 mM, and 0.5 mL penicillin/streptomycin (Invitrogen #15140) to a final concentration of 50 U/mL each.

E. DNA Purification

DNA purification was conducted as described in Ji et al., PNAS 112(12):3841-3846 (2015) Supporting Information, which is hereby incorporated by reference in its entirety. One milliliter of 2.5 M glycine was added to each plate of fixed cells and incubated for 5 minutes to quench the formaldehyde. The cells were washed twice with PBS. The cells were pelleted at 1,300 g for 5 minutes at 4° C. Then, 4×10⁷ cells were collected in each tube. The cells were lysed gently with 1 mL of ice-cold Nonidet P-40 lysis buffer containing protease inhibitor on ice for 5 minutes (buffer recipes are provided below). The cell lysate was layered on top of 2.5 volumes of sucrose cushion made up of 24% (wt/vol) sucrose in Nonidet P-40 lysis buffer. This sample was centrifuged at 18,000 g for 10 minutes at 4° C. to isolate the nuclei pellet (the supernatant represented the cytoplasmic fraction). The nuclei pellet was washed once with PBS/1 mM EDTA. The nuclei pellet was resuspended gently with 0.5 mL glycerol buffer followed by incubation for 2 minutes on ice with an equal volume of nuclei lysis buffer. The sample was centrifuged at 16,000 g for 2 minutes at 4° C. to isolate the chromatin pellet (the supernatant represented the nuclear soluble fraction). The chromatin pellet was washed twice with PBS/1 mM EDTA. The chromatin pellet was stored at −80° C.

The Nonidet P-40 lysis buffer contained 10 mM Tris.HCl (pH 7.5), 150 mM NaCl, and 0.05% Nonidet P-40. The glycerol buffer contained 20 mM Tris.HCl (pH 7.9), 75 mM NaCl, 0.5 mM EDTA, 0.85 mM DTT, and 50% (vol/vol) glycerol. The nuclei lysis buffer contained 10 mM Hepes (pH 7.6), 1 mM DTT, 7.5 mM MgCl₂, 0.2 mM EDTA, 0.3 M NaCl, 1 M urea, and 1% Nonidet P-40.

F. Chromatin Immunoprecipitation Sequencing (ChIP-seq)

ChIP-seq was performed using the following protocol for primary hepatocytes and HepG2 cells to determine the composition and confirm the location of signaling centers.

i. Cell Cross-Linking

2×10⁷ cells were used for each run of ChIP-seq. Two ml of fresh 11% formaldehyde (FA) solution was added to 20 ml media on 15 cm plates to reach a 1.1% final concentration. Plates were swirled briefly and incubated at room temperature (RT) for 15 minutes. At the end of incubation, the FA was quenched by adding 1 ml of 2.5M Glycine to plates and incubating for 5 minutes at RT. The media was discarded to a 1 L beaker, and cells were washed twice with 20 ml ice-cold PBS. PBS (10 ml) was added to plates, and cells were scraped off the plate. The cells were transferred to 15 ml conical tubes, and the tubes were placed on ice. Plates were washed with an additional 4 ml of PBS and combined with cells in 15 ml tubes. Tubes were centrifuged for 5 minutes at 1,500 rpm at 4° C. in a tabletop centrifuge. PBS was aspirated, and the cells were flash frozen in liquid nitrogen. Pellets were stored at −80° C. until ready to use.

ii. Pre-Block Magnetic Beads

Thirty μl Protein G beads (per reaction) were added to a 1.5 ml Protein LoBind Eppendorf tube. The beads were collected by magnet separation at RT for 30 seconds. Beads were washed 3 times with 1 ml of blocking solution by incubating beads on a rotator at 4° C. for 10 minutes and collecting the beads with the magnet. Five μg of an antibody was added to the 250 μl of beads in block solution. The mix was transferred to a clean tube, and rotated overnight at 4° C. On the next day, buffer containing antibodies was removed, and beads were washed 3 times with 1.1 ml blocking solution by incubating beads on a rotator at 4° C. for 10 minutes and collecting the beads with the magnet. Beads were resuspended in 50 μl of block solution and kept on ice until ready to use.

iii. Cell Lysis, Genomic Fragmentation, and Chromatin Immunoprecipitation

COMPLETE® protease inhibitor cocktail was added to lysis buffer 1 (LB1) before use. One tablet was dissolved in 1 ml of H₂O for a 50× solution. The cocktail was stored in aliquots at −20° C. Cells were resuspended in each tube in 8 ml of LB1 and incubated on a rotator at 4° C. for 10 minutes. Nuclei were spun down at 1,350 g for 5 minutes at 4° C. LB1 was aspirated, and cells were resuspended in each tube in 8 ml of LB2 and incubated on a rotator at 4° C. for 10 minutes.

A COVARIS® E220EVOLUTION™ ultrasonicator was programmed per the manufacturer's recommendations for high cell numbers. HepG2 cells were sonicated for 12 minutes, and primary hepatocyte samples were sonicated for 10 minutes. Lysates were transferred to clean 1.5 ml Eppendorf tubes, and the tubes were centrifuged at 20,000 g for 10 minutes at 4° C. to pellet debris. The supernatant was transferred to a 2 ml Protein LoBind Eppendorf tube containing pre-blocked Protein G beads with pre-bound antibodies. Fifty μl of the supernatant was saved as input. Input material was kept at −80° C. until ready to use. Tubes were rotated with beads overnight at 4° C.

iv. Wash, Elution, and Cross-Link Reversal

All washing steps were performed by rotating tubes for 5 minutes at 4° C. The beads were transferred to clean Protein LoBind Eppendorf tubes with every washing step. Beads were collected in 1.5 ml Eppendorf tube using a magnet. Beads were washed twice with 1.1 ml of sonication buffer. The magnetic stand was used to collect magnetic beads. Beads were washed twice with 1.1 ml of wash buffer 2, and the magnetic stand was used again to collect magnetic beads. Beads were washed twice with 1.1 ml of wash buffer 3. All residual Wash buffer 3 was removed, and beads were washed once with 1.1 ml TE+0.2% Triton X-100 buffer. Residual TE+0.2% Triton X-100 buffer was removed, and beads were washed twice with TE buffer for 30 seconds each time. Residual TE buffer was removed, and beads were resuspended in 300 μl of ChIP elution buffer. Two hundred fifty μl of ChIP elution buffer was added to 50 μl of input, and the tubes were rotated with beads 1 hour at 65° C. Input sample was incubated overnight at 65° C. oven without rotation. Tubes with beads were placed on a magnet, and the eluate was transferred to a fresh DNA LoBind Eppendorf tube. The eluate was incubated overnight at 65° C. oven without rotation

v. Chromatin Extraction and Precipitation

Input and immunoprecipitant (IP) samples were transferred to fresh tubes, and 300 μl of TE buffer was added to IP and Input samples to dilute SDS. RNase A (20 mg/ml) was added to the tubes, and the tubes were incubated at 37° C. for 30 minutes. Following incubation, 3 μl of 1M CaCl₂ and 7 μl of 20 mg/ml Proteinase K were added, and incubated 1.5 hours at 55° C. MaXtract High Density 2 ml gel tubes (Qiagen) were prepared by centrifugation at full speed for 30 seconds at RT. Six hundred μl of phenol/chloroform/isoamyl alcohol was added to each proteinase K reaction and transferred in about 1.2 ml mixtures to the MaXtract tubes. Tubes were spun at 16,000 g for 5 minutes at RT. The aqueous phase was transferred to two clean DNA LoBind tubes (300 μl in each tube), and 1.5 μl glycogen, 30 μl of 3M sodium acetate, and 900 μl ethanol were added. The mixture was precipitated overnight at −20° C. or for 1 hour at −80° C., and spun down at maximum speed for 20 minutes at 4° C. The ethanol was removed, and pellets were washed with 1 ml of 75% ethanol by spinning tubes down at maximum speed for 5 minutes at 4° C. Remnants of ethanol were removed, and pellets were dried for 5 min at RT. Twenty-five μl of H₂O was added to each immunoprecipitant (IP) and input pellet, left standing for 5 minutes, and vortexed briefly. DNA from both tubes was combined to obtain 50 μl of IP and 50 μl of input DNA for each sample. One μl of this DNA was used to measure the amount of pulled down DNA using Qubit dsDNA HS assay (ThermoFisher, #Q32854). The total amount of immunoprecipitated material ranged from several ng (for TFs) to several hundred ng (for chromatin modifications). Six μl of DNA was analyzed using qRT-PCR to determine enrichment. The DNA was diluted if necessary. If enrichment was satisfactory, the rest was used for library preparation for DNA sequencing.

vi. Library Preparation for DNA Sequencing

Libraries were prepared using NEBNext Ultra II DNA library prep kit for Illumina (NEB, #E7645) using NEBNext Multiplex Oligos for Illumina (NEB, #6609S) according to manufacturer's instructions with the following modifications. The remaining ChIP sample (about 43 μl) and lug of input samples for library preparations were brought up the volume of 50 μl before the End Repair portion of the protocol. End Repair reactions were run in a PCR machine with a heated lid in a 96-well semi-skirted PCR plate (ThermoFisher, #AB1400) sealed with adhesive plate seals (ThermoFisher, #AB0558) leaving at least one empty well in-between different samples. Undiluted adapters were used for input samples, 1:10 diluted adapters for 5-100 ng of ChIP material, and 1:25 diluted adapters for less than 5 ng of ChIP material. Ligation reactions were run in a PCR machine with the heated lid off. Adapter ligated DNA was transferred to clean DNA LoBind Eppendorf tubes, and the volume was brought to 96.5 μl using H₂O.

200-600 bp ChIP fragments were selected using SPRIselect magnetic beads (Beckman Coulter, #B23317). Thirty μl of the beads were added to 96.5 μl of ChIP sample to bind fragments that are longer than 600 bp. The shorter fragments were transferred to a fresh DNA LoBind Eppendorf tube. Fifteen μl of beads were added to bind the DNA longer than 200 bp, and beads were washed with DNA twice using freshly prepared 75% ethanol. DNA was eluted using 17 μl of 0.1× TE buffer. About 15 μl was collected.

Three μl of size-selected Input sample and all (15 μl) of the ChIP sample was used for PCR. The amount of size-selected DNA was measured using a Qubit dsDNA HS assay. PCR was run for 7 cycles of for Input and ChIP samples with about 5-10 ng of size-selected DNA, and 12 cycles with less than 5 ng of size-selected DNA. One-half of the PCR product (25 μl) was purified with 22.5 μl of AMPure XP beads (Beckman Coulter, #A63880) according to the manufacturer's instructions. PCR product was eluted with 17 μl of 0.1× TE buffer, and the amount of PCT product was measured using Qubit dsDNA HS assay. An additional 4 cycles of PCR were run for the second half of samples with less than 5 ng of PCR product, DNA was purified using 22.5 μl of AMPure XP beads. The concentration was measured to determine whether there was an increased yield. Both halves were combined, and the volume was brought up to 50 μl using H₂O.

A second round of purifications of DNA was run using 45 μl of AMPure XP beads in 17 μl of 0.1× TE, and the final yield was measured using Qubit dsDNA HS assay. This protocol produces from 20 ng to 1 mg of PCR product. The quality of the libraries was verified by diluting 1 μl of each sample with H₂O if necessary using the High Sensitivity BioAnalyzer DNA kit (Agilent, #5067-4626) based on manufacturer's recommendations.

vii. Reagents

11% Formaldehyde Solution (50 mL) contained 14.9 ml of 37% formaldehyde (final conc. 11%), 1 ml of 5M NaCl (final conc. 0.1 M), 100 μl of 0.5M EDTA (pH 8) (final conc. 1 mM), 50 μl of 0.5M EGTA (pH 8) (final conc. 0.5 mM), and 2.5 ml 1M Hepes (pH 7.5) (final conc. 50 mM).

Block Solution contained 0.5% BSA (w/v) in PBS and 500 mg BSA in 100 ml PBS. Block solution may be prepared up to about 4 days prior to use.

Lysis buffer 1 (LB1) (500 ml) contained 25 ml of 1 M Hepes-KOH, pH 7.5; 14 ml of 5M NaCl; 1 ml of 0.5M EDTA, pH 8.0; 50 ml of 100% Glycerol solution; 25 ml of 10% NP-40; and 12.5 ml of 10% Triton X-100. The pH was adjusted to 7.5. The buffer was sterile-filtered, and stored at 4° C. The pH was re-checked immediately prior to use.

Lysis buffer 2 (LB2) (1000 ml) contained 10 ml of 1 M Tris-HCL, pH 8.0; 40 ml of 5 M NaCl; 2 ml of 0.5M EDTA, pH 8.0; and 2 ml of 0.5M EGTA, pH 8.0. The pH was adjusted to 8.0. The buffer was sterile-filtered, and stored at 4° C. The pH was re-checked immediately prior to use.

Sonication buffer (500 ml) contained 25 ml of 1M Hepes-KOH, pH 7.5; 14 ml of 5M NaCl; 1 ml of 0.5M EDTA, pH 8.0; 50 ml of 10% Triton X-100; 10 ml of 5% Na-deoxycholate; and 5 ml of 10% SDS. The pH was adjusted to 7.5. The buffer was sterile-filtered, and stored at 4° C. The pH was re-checked immediately prior to use.

Proteinase inhibitors were included in the LB1, LB2, and Sonication buffer.

Wash Buffer 2 (500 ml) contained 25 ml of 1M Hepes-KOH, pH 7.5; 35 ml of 5M NaCl; 1 ml of 0.5M EDTA, pH 8.0; 50 ml of 10% Triton X-100; 10 ml of 5% Na-deoxycholate; and 5 ml of 10% SDS. The pH was adjusted to 7.5. The buffer was sterile-filtered, and stored at 4° C. The pH was re-checked immediately prior to use.

Wash Buffer 3 (500 ml) contained 10 ml of 1M Tris-HCL, pH 8.0; 1 ml of 0.5M EDTA, pH 8.0; 125 ml of 1M LiCl solution; 25 ml of 10% NP-40; and 50 ml of 5% Na-deoxycholate. The pH was adjusted to 8.0. The buffer was sterile-filtered, and stored at 4° C. The pH was re-checked immediately prior to use.

ChIP elution Buffer (500 ml) contained 25 ml of 1 M Tris-HCL, pH 8.0; 10 ml of 0.5M EDTA, pH 8.0; 50 ml of 10% SDS; and 415 ml of ddH₂O. The pH was adjusted to 7.5. The buffer was sterile-filtered, and stored at 4° C. The pH was re-checked immediately prior to use.

G. Analysis of ChIP-seq Results

All pass filter reads from each sample were trimmed of sequencing adapters using trim_galore 0.4.4 with default options. Trimmed reads were mapped against the human genome (assembly GRCh38/GCA_000001405.15 “no alt” analysis set merged with hs38d1/GCA_000786075.2) using bwa version 0.7.15 (Li (2013) arXiv:1303.3997v1) with default parameters. Aligned read duplicates were assessed using picard 2.9.0 (http://broadinstitute.hithub.io/picard) and reads with a MAPQ<20 or those matching standard SAM flags 0x1804 were discarded. Standard QC were applied (read integrity, mapping statistics, library complexity, fragment bias) to remove unsatisfactory samples. Enriched ChIP-seq peaks were identified by comparing samples against whole cell extract controls using MACS2 version 2.1.0 (Zhang et al., Genome Biol. (2008) 9(9):R137), with significant peaks selected as those with an adjusted p-value <0.01. Peaks overlapping known repetitive “blacklist” regions (ENCODE Project Consortium, Nature (2012) 489(7414:57-74) were discarded. ChIP-seq signals were also normalized by read depth and visualized using the UCSC browser.

H. RNA-seq

This protocol is a modified version of the following protocols: MagMAX mirVana Total RNA Isolation Kit User Guide (Applied Biosystems #MAN0011131 Rev B.0), NEBNext Poly(A) mRNA Magnetic Isolation Module (E7490), and NEBNext Ultra Directional RNA Library Prep Kit for Illumina (E7420) (New England Biosystems #E74901).

The MagMAX mirVana kit instructions (the section titled “Isolate RNA from cells” on pages 14-17) were used for isolation of total RNA from cells in culture. Two hundred μl of Lysis Binding Mix was used per well of the multiwell plate containing adherent cells (usually a 24-well plate).

For mRNA isolation and library prep, the NEBNext Poly(A) mRNA Magnetic Isolation Module and Directional Prep kit was used. RNA isolated from cells above was quantified, and prepared in 500 μg of each sample in 50 μl of nuclease-free water. This protocol may be run in microfuge tubes or in a 96-well plate.

The 80% ethanol was prepared fresh, and all elutions are done in 0.1× TE Buffer. For steps requiring Ampure XP beads, beads were at room temperature before use. Sample volumes were measured first and beads were pipetted. Section 1.9B (not 1.9A) was used for NEBNext Multiplex Oligos for Illumina (#E6609). Before starting the PCR enrichment, cDNA was quantified using the Qubit (DNA High Sensitivity Kit, ThermoFisher #Q32854). The PCR reaction was run for 12 cycles.

After purification of the PCR Reaction (Step 1.10), the libraries were quantified using the Qubit DNA High Sensitivity Kit. 1 μl of each sample were diluted to 1-2 ng/μl to run on the Bioanalyzer (DNA High Sensitivity Kit, Agilent #5067-4626). If Bioanalyzer peaks were not clean (one narrow peak around 300 bp), the AMPure XP bead cleanup step was repeated using a 0.9× or 1.0× beads:sample ratio. Then, the samples were quantified again with the Qubit, and run again on the Bioanalyzer (1-2 ng/μl).

Nuclear RNA from INTACT-purified nuclei or whole neocortical nuclei was converted to cDNA and amplified with the Nugen Ovation RNA-seq System V2. Libraries were sequenced using the Illumina HiSeq 2500.

I. RNA-seq Data Analysis

All pass filter reads from each sample were mapped against the human genome (assembly GRCh38/GCA_000001405.15 “no alt” analysis set merged with hs38d1/GCA_000786075.2) using two pass mapping via STAR version 2.5.3a (alignment parameters alignIntronMin=20; alignIntronMax=1000000; outFilterMismatchNmax=999; outFilterMismatchNoverLmax=0.05; outFilterType=BySJout; outFilterMultimapNmax=20; alignSJoverhangMin=8; alignSJDBoverhangMin=1; alignMatesGapMax=1000000) (Dobin et al., Bioinformatics (2012) 29(1):15-21). Genomic alignments were converted to transcriptome alignments based on reference transcript annotations from the Human GENCODE Gene Set release 24 (Harrow et al., Genome Res. (2012) 22(9): 1760-1774). Using unique and multimapped transcriptomic alignments, gene-level abundance estimates were computed using RSEM version 1.3.0 (Li and Dewey, BMC Bioinformatics (2011) 12:323) in a strand-aware manner, and including confidence interval sampling calculations, to arrive at posterior mean estimates (PME) of abundances (counts and normalized FPKM—fragments per kilobase of exon per million mapped fragments) from the underlying Bayesian model. Standard QC were applied (read integrity, mapping statistics, library complexity, fragment bias) to remove unsatisfactory samples. Differential gene expression was computed using the negative binomial model implemented by DESeq2 version 1.16.1 (Love et al., Genome Biol. (2014) 15(12):550). Log 2 fold change and significance values were computed using PME count data (with replicates explicitly modeled versus pan-experiment controls), median ratio normalized, using maximum likelihood estimation rather than maximum a posteriori, and disabling the use of Cook's distance cutoff when determining acceptable adjusted p-values. Significantly differential genes were assigned as those with an adjusted p-value <0.01, a log 2 fold change of >=1 or <=−1, and at least one replicate with PME FPKM >=1. RNA-seq signals were also normalized by read depth and visualized using the UCSC browser.

J. ATAC-seq

Hepatocytes were seeded overnight, then the serum and other factors were removed. After 2-3 hours, the cells were treated with the compound and incubated overnight. The cells were harvested and the nuclei were prepared for the transposition reaction. 50,000 bead bound nuclei were transposed using Tn5 transposase (Illumina FC-121-1030) as described in Mo et al., 2015, Neuron 86, 1369-1384, which is hereby incorporated by reference in its entirety. After 9-12 cycles of PCR amplification, libraries were sequenced on an Illumina HiSeq 2000. PCR was performed using barcoded primers with extension at 72° C. for 5 minutes, PCR, then the final PCR product was sequenced.

All obtained reads from each sample were trimmed using trim_galore 0.4.1 requiring Phred score ≥20 and read length ≥30 for data analysis. The trimmed reads were mapped against the human genome (hg19 build) using Bowtie2 (version 2.2.9) with the parameters: -t -q -N 1 -L 25 -X 2000 no-mixed no-discordant. All unmapped reads, non-uniquely mapped reads and PCR duplicates were removed. All the ATAC-seq peaks were called using MACS2 with the parameters --nolambda -nomodel -q 0.01 --SPMR. The ATAC-seq signal was visualized in the UCSC genome browser. ATAC-seq peaks that were at least 2 kb away from annotated promoters (RefSeq, Ensemble and UCSC Known Gene databases combined) were selected as distal ATAC-seq peaks.

K. qRT-PCR

qRT-PCR was performed as described in North et al., PNAS, 107(40) 17315-17320 (2010), which is hereby incorporated by reference in its entirety. Prior to qRT-PCR analysis, cell medium was removed and replaced with RLT Buffer for RNA extraction (Qiagen RNeasy 96 QIAcube HT Kit Cat #74171). Cells were processed for RNA extraction using RNeasy 96 kit (Qiagen Cat #74182). For Taqman qPCR analysis, cDNA was synthesized using High-Capacity cDNA Reverse Transcription Kit (ThermoFisher Scientific cat:4368813 or 4368814) according to manufacturer instructions. qRT-PCR was performed with cDNA using the iQ5 Multicolor rtPCR Detection system from BioRad with 60° C. annealing. Samples were amplified using Taqman probes from ThermoFisher.

Analysis of the fold changes in expression as measured by qRT-PCR were performed using the technique below. The control was DMSO, and the treatment was the selected compound (CPD). The internal control was GAPDH or B-Actin (or otherwise indicated), and the gene of interest is the target. First, the averages of the 4 conditions were calculated for normalization: DMSO:GAPDH, DMSO:Target, CPD:GAPDH, and CPD:Target. Next, the ACT of both control and treatment were calculated to normalize to internal control (GAPDH) using (DMSO:Target)−(DMSO:GAPDH)=ΔCT control and (CPD:Target)—(CPD:GAPDH)=ΔCT experimental. Then, the ΔΔCT was calculated by ΔCT experimental−ΔCT control. The Expression Fold Change (RQ) was calculated by 2-(ΔΔCT) (2-fold expression change was shown by RNA-Seq results provided herein).

In some examples, RQ Min and RQ Max values are also reported. RQ Min and RQ Max are the minimum and maximum relative levels of gene expression in the test samples, respectively. They were calculated using the confidence level set in the analysis settings and the confidence level was set to one standard deviation (SD). These values were calculated using standard deviation as follows: RQ Min=2−(ΔΔCT−SD); and RQ Max=2−(ΔΔCT+SD).

L. Chromatin Interaction Analysis by Paired-End Tag Sequencing (ChIA-PET)

ChIA-PET is performed as previously described in Chepelev et al. (2012) Cell Res. 22, 490-503; Fullwood et al. (2009) Nature 462, 58-64; Goh et al. (2012) J. Vis. Exp., http://dx.doi.org/10.3791/3770; Li et al. (2012) Cell 148, 84-98; and Dowen et al. (2014) Cell 159, 374-387, which are each hereby incorporated by reference in their entireties. Briefly, embryonic stem (ES) cells (up to 1×10⁸ cells) are treated with 1% formaldehyde at room temperature for 20 minutes and then neutralized using 0.2M glycine. The crosslinked chromatin is fragmented by sonication to size lengths of 300-700 bp. The anti-SMC1 antibody (Bethyl, A300-055A) is used to enrich SMC1-bound chromatin fragments. A portion of ChIP DNA is eluted from antibody-coated beads for concentration quantification and for enrichment analysis using quantitative PCR. For ChIA-PET library construction ChIP DNA fragments are end-repaired using T4 DNA polymerase (NEB). ChIP DNA fragments are divided into two aliquots and either linker A or linker B is ligated to the fragment ends. The two linkers differ by two nucleotides which are used as a nucleotide barcode (Linker A with CG; Linker B with AT). After linker ligation, the two samples are combined and prepared for proximity ligation by diluting in a 20 ml volume to minimize ligations between different DNA-protein complexes. The proximity ligation reaction is performed with T4 DNA ligase (Fermentas) and incubated without rocking at 22° C. for 20 hours. During the proximity ligation DNA fragments with the same linker sequence are ligated within the same chromatin complex, which generated the ligation products with homodimeric linker composition. However, chimeric ligations between DNA fragments from different chromatin complexes could also occur, thus producing ligation products with heterodimeric linker composition. These heterodimeric linker products are used to assess the frequency of nonspecific ligations and were then removed.

i. Day 1

The cells were crosslinked as described for ChIP. Frozen cell pellets were stored in the −80° C. freezer until ready to use. This protocol required at least 3×10⁸ cells frozen in six 15 ml Falcon tubes (50 million cells per tube). Six 100 μl Protein G Dynabeads (for each ChIA-PET sample) were added to six 1.5 ml Eppendorf tubes on ice. Beads were washed three times with 1.5 ml Block solution, and incubated end over end at 4° C. for 10 minutes between each washing step to allow for efficient blocking. Protein G Dynabeads were resuspended in 250 μl of Block solution in each of six tubes and 10 μg of SMC1 antibody (Bethyl A300-055A) is added to each tube. The bead-antibody mixes were incubated at 4° C. end-over-end overnight.

ii. Day 2

Beads were washed three times with 1.5 ml Block solution to remove unbound IgG and incubated end-over-end at 4° C. for 10 minutes each time. Smcl-bound beads were resuspended in 100 μl of Block solution and stored at 4° C. Final lysis buffer 1 (8 ml per sample) was prepared by adding 50× Protease inhibitor cocktail solution to Lysis buffer 1 (LB1) (1:50). Eight ml of Final lysis buffer 1 was added to each frozen cell pellet (8 ml per sample×6). The cells were thoroughly resuspended and thawed on ice by pipetting up and down. The cell suspension was incubated again end-over-end for 10 minutes at 4° C. The suspension was centrifuged at 1,350 g for 5 minutes at 4° C. Concurrently, Final lysis buffer 2 (8 ml per sample) was prepared by adding 50× Protease inhibitor cocktail solution to lysis buffer 2 (LB2) (1:50)

After centrifugation, the supernatant was discarded, and the nuclei were thoroughly resuspended in 8 ml Final lysis buffer 2 by pipetting up and down. The cell suspension was incubated end-over-end for 10 minutes at 4° C. The suspension was centrifuged at 1,350 g for 5 minutes at 4° C. During incubation and centrifugation, the Final sonication buffer (15 ml per sample) was prepared by adding 50× Protease inhibitor cocktail solution to sonication buffer (1:50). The supernatant was discarded, and the nuclei were fully resuspended in 15 ml Final sonication buffer by pipetting up and down. The nuclear extract was extracted to fifteen 1 ml Covaris Evolution E220 sonication tubes on ice. An aliquot of 10 μl was used to check the size of unsonicated chromatin on a gel.

A Covaris sonicator was programmed according to manufacturer's instructions (12 minutes per 20 million cells=12×15=3 hours). The samples were sequentially sequenced as described above. The goal was to break chromatin DNA to 200-600 bp. If sonication fragments were too big, false positives became more frequent. The sonicated nuclear extract was dispensed into 1.5 ml Eppendorf tubes. 1.5 ml samples were centrifuged at full speed at 4° C. for 10 minutes. Supernatant (SNE) was pooled into a new pre-cooled 50 ml Falcon tube, and brought to a volume of 18 ml with sonication buffer. Two tubes of 50 μl were taken as input and to check the size of fragments. 250 μl of ChIP elution buffer was added and reverse crosslinking occurs at 65° C. overnight in the oven After reversal of crosslinking, the size of sonication fragments was determined on a gel.

Three ml of sonicated extract was added to 100 μl Protein G beads with SMC1 antibodies in each of six clean 15 ml Falcon tubes. The tubes containing SNE-bead mix were incubated end-over-end at 4° C. overnight (14 to 18 hours).

iii. Day 3

Half the volume (1.5 ml) of the SNE-bead mix was added to each of six pre-chilled tubes and SNE was removed using a magnet. The tubes were sequentially washed as follows: 1) 1.5 ml of Sonication buffer was added, the beads were resuspended and rotated for 5 minutes at 4° C. for binding, then the liquid was removed (step performed twice); 2) 1.5 ml of high-salt sonication buffer was added, and the beads were resuspended and rotated for 5 minutes at 4° C. for binding, then the liquid was removed (step performed twice); 3) 1.5 ml of high-salt sonication buffer was added, and the beads were resuspended and rotated for 5 minutes at 4° C. for binding, then the liquid was removed (step performed twice); 4) 1.5 ml of LiCl buffer was added, and the cells were resuspended and incubated end-over-end for 5 minutes for binding, then the liquid was removed (step performed twice); 5) 1.5 ml of 1× TE+0.2% Triton X-100 was used to wash the cells for 5 minutes for binding, then the liquid was removed; and 1.5 ml of ice-cold TE Buffer was used to wash the cells for 30 seconds for binding, then the liquid was removed (step performed twice). Beads from all six tubes were sequentially resuspended in beads in one 1,000 ul tube of 1× ice-cold TE buffer.

ChIP-DNA was quantified using the following protocol. Ten percent of beads (by volume), or 100 μl, were transferred into a new 1.5 ml tube, using a magnet. Beads were resuspended in 300 μl of ChIP elution buffer and the tube was rotated with beads for 1 hour at 65° C. The tube with beads was placed on a magnet and the eluate was transferred to a fresh DNA LoBind Eppendorf tube. The eluate was incubated overnight at 65° C. oven without rotating Immuno-precipitated samples were transferred to fresh tubes, and 300 μl of TE buffer was added to the immuno-precipitants and Input samples to dilute. Five μl of RNase A (20 mg/ml) was added, and the tube was incubated at 37° C. for 30 minutes.

Following incubation, 3 μl of 1M CaCl₂ and 7 μl of 20 mg/ml Proteinase K was added to the tube and incubated 1.5 hours at 55° C. MaXtract High Density 2 ml gel tubes (Qiagen) were prepared by centrifuging them at full speed for 30 seconds at RT. 600 μl of phenol/chloroform/isoamyl alcohol was added to each proteinase K reaction. About 1.2 ml of the mixtures was transferred to the MaXtract tubes. Tubes were spun at 16,000 g for 5 minutes at RT. The aqueous phase was transferred to two clean DNA LoBind tubes (300 μl in each tube), and 1 μl glycogen, 30 μl of 3M sodium acetate, and 900 μl ethanol was added. The mixture was allowed to precipitate overnight at −20° C. or for 1 hour at −80° C.

The mixture was spun down at maximum speed for 20 minutes at 4° C., ethanol was removed, and the pellets were washed with 1 ml of 75% ethanol by spinning tubes down at maximum speed for 5 minutes at 4° C. All remnants of ethanol were removed, and pellets were dried for 5 minutes at RT. H₂O is added to each tube. Each tube was allowed to stand for 5 minutes, and vortexed briefly. DNA from both tubes was combined to obtain 50 μl of IP and 100 μl of Input DNA.

The amount of DNA collected was quantitated by ChIP using Qubit (Invitrogen #Q32856). One μl intercalating dye was combined with each measure 1 μl of sample. Two standards that come with the kit were used. DNA from only 10% of the beads was measured. About 400 ng of chromatin in 900 μl of bead suspension was obtained with a good enrichment at enhancers and promoters as measured by qPCR.

iv. Day 3 or 4

End-blunting of ChIP-DNA was performed on the beads using the following protocol. The remaining chromatin/beads were split by pipetting, and 450 μl of bead suspension was aliquoted into 2 tubes. Beads were collected on a magnet. Supernatant was removed, and then the beads were resuspended in the following reaction mix: 70 μl 10× NEB buffer 2.1 (NEB, M0203L), 7 μl 10 mM dNTPs, 615.8 μl dH₂0, and 7.2 μl of 3 U/μl T4 DNA Polymerase (NEB, M0203L). The beads were incubated at 37° C. with rotation for 40 minutes. Beads were collected with a magnet, then the beads were washed 3 times with 1 ml ice-cold ChIA-PET Wash Buffer (30 seconds per each wash).

On-Bead A-tailing was performed by preparing Klenow (3′ to 5′exo-) master mix as stated below: 70 μl 10× NEB buffer 2, 7 μl 10 mM dATP, 616 μl dH20, and 7 μl of 3 U/μl Klenow (3′ to 5′exo-) (NEB, M0212L). The mixture was incubated at 37° C. with rotation for 50 minutes. Beads were collected with a magnet, then beads were washed 3 times with 1 ml of ice-cold ChIA-PET Wash Buffer (30 seconds per each wash).

Linkers were thawed gently on ice. Linkers were mixed well with water gently by pipetting, then with PEG buffer, then gently vortexed. Then, 1394 μl of master mix and 6 μl of ligase was added per tube and mixed by inversion. Parafilm was put on the tube, and the tube was incubated at 16° C. with rotation overnight (at least 16 hours). The biotinylated linker was ligated to ChIP-DNA on beads by setting up the following reaction mix and adding reagents in order: 1110 μl dH₂0, 4 μl 200 ng/μl biotinylated bridge linker, 280 μl 5× T4 DNA ligase buffer with PEG (Invitrogen), and 6 μl 30 U/μl T4 DNA ligase (Fermentas).

v. Day 5

Exonuclease lambda/Exonuclease I On-Bead digestion was performed using the following protocol. Beads were collected with a magnet and washed 3 times with 1 ml of ice-cold ChIA-PET Wash Buffer (30 seconds per each wash). The Wash buffer was removed from beads, then resuspended in the following reaction mix: 70 μl 10× lambda nuclease buffer (NEB, M0262L), 618 μl nuclease-free dH20, 6 μl 5 U/μl Lambda Exonuclease (NEB, M0262L), and 6 μl Exonuclease I (NEB, M0293L). The reaction was incubated at 37° C. with rotation for 1 hour. Beads were collected with a magnet, and beads are washed 3 times with 1 ml ice-cold ChIA-PET Wash Buffer (30 seconds per each wash).

Chromatin complexes were eluted off the beads by removing all residual buffer and resuspending the beads in 300 μl of ChIP elution buffer. The tube with beads was rotated 1 hour at 65° C. The tube was placed on a magnet and the eluate was transferred to a fresh DNA LoBind Eppendorf tube. The eluate was incubated overnight at 65° C. in an oven without rotating.

vi. Day 6

The eluted sample was transferred to a fresh tube and 300 μl of TE buffer was added to dilute the SDS. Three μl of RNase A (30 mg/ml) was added to the tube, and the mixture was incubated at 37° C. for 30 minutes. Following incubation, 3 μl of 1M CaCl₂ and 7 μl of 20 mg/ml Proteinase K was added, and the tube was incubated again for 1.5 hours at 55° C. MaXtract High Density 2 ml gel tubes (Qiagen) were used and the material was precipitated and pellated by centrifuging the tubes at full speed for 30 seconds at RT. Six hundred μl of phenol/chloroform/isoamyl alcohol was added to each proteinase K reaction, and about 1.2 ml of the mixture was transferred to the MaXtract tubes. Tubes were spun at 16,000 g for 5 minutes at RT.

The aqueous phase was transferred to two clean DNA LoBind tubes (300 μl in each tube), and 1 μl glycogen, 30 μl of 3M sodium acetate, and 900 μl ethanol was added. The mixture was precipitated for 1 hour at −80° C. The tubes were spun down at maximum speed for 30 minutes at 4° C., and the ethanol was removed. The pellets were washed with 1 ml of 75% ethanol by spinning tubes down at maximum speed for 5 minutes at 4° C. Remnants of ethanol were removed, and the pellets were dried for 5 minutes at RT. Thirty μl of H₂O was added to the pellet and allowed to stand for 5 minutes. The pellet mixture was vortexed briefly, and spun down to collect the DNA.

Qubit and DNA High Sensitivity ChIP were performed to quantify and assess the quality of proximity ligated DNA products. About 120 ng of the product was obtained.

vii. Day 7

Components for Nextera tagmentation were then prepared. One hundred ng of DNA was divided into four 25 μl reactions containing 12.5 μl 2× Tagmentation buffer (Nextera), 1 μl nuclease-free dH₂0, 2.5 μl Tn5 enzyme(Nextera), and 9 μl DNA (25 ng). Fragments of each of the reactions were analyzed on a Bioanalyzer for quality control.

The reactions were incubated at 55° C. for 5 minutes, then at 10° C. for 10 minutes. Twenty-five μl of H₂O was added, and tagmented DNA was purified using Zymo columns. Three hundred fifty μl of Binding Buffer was added to the sample, and the mixture was loaded into a column and spun at 13,000 rpm for 30 seconds. The flow through was re-applied and the columns were spun again. The columns were washed twice with 200 μl of wash buffer and spun for 1 minute to dry the membrane. The column was transferred to a clean Eppendorf tube and 25 μl of Elution buffer was added. The tube was spun down for 1 minute. This step was repeated with another 25 μl of elution buffer. All tagmented DNA was combined into one tube.

ChIA-PETs were immobilized on Streptavidin beads using the following steps. 2× B&W Buffer (40 ml) was prepared as follows for coupling of nucleic acids: 400 μl 1M Tris-HCl pH 8.0 (10 mM final), 80 μl 1M EDTA (1 mM final), 16 ml 5M NaCl (2M final), and 23.52 ml dH₂O. 1× B&W Buffer (40 ml total) was prepared by adding 20 ml dH₂O to 20 ml of the 2× B&W Buffer.

MyOne Streptavidin Dynabeads M-280 were allowed to come to room temperature for 30 minutes, and 30 μl of beads were transferred to a new 1.5 ml tube. Beads were washed with 150 μl of 2× B&W Buffer twice. Beads were resuspended in 100 μl of iBlock buffer (Applied Biosystems), and mixed. The mixture was incubated at RT for 45 minutes on a rotator.

I-BLOCK Reagent was prepared to contain: 0.2% I-Block reagent (0.2 g), 1× PBS or 1× TBS (10 ml 10× PBS or 10× TBS), 0.05% Tween-20 (50 μl), and H₂O to 100 ml. 10× PBS and I-BLOCK reagent was added to H₂O, and the mixture was microwaved for 40 seconds (not allowed to boil), then stirred. Tween-20 was added after the solution is cooled. The solution remained opaque, but particles were dissolved. The solution was cooled to RT for use.

During incubation of beads, 500 ng of sheared genomic DNA was added to 50 μl of H₂O and 50 μl of 2× B&W Buffer. When the beads finished incubating with the iBLOCK buffer, they were washed twice with 200 μl of 1× B&W buffer. The wash buffer was discarded, and 100 μl of the sheared genomic DNA was added. The mixture was incubated with rotation for 30 minutes at RT. The beads were washed twice with 200 μl of 1× B&W buffer. Tagmented DNA was added to the beads with an equal volume of 2× B&W buffer and incubated for 45 minutes at RT with rotation. The beads were washed 5 times with 500 μl of 2×SSC/0.5% SDS buffer (30 seconds each time) followed by 2 washes with 500 ml of 1× B&W Buffer and incubating each after wash for 5 minutes at RT with rotation. The beads were washed once with 100 μl elution buffer (EB) from a Qiagen Kit by resuspending beads gently and putting the tube on a magnet. The supernatant was removed from the beads, and they were resuspended in 30 μl of EB.

A paired end sequencing library was constructed on beads using the following protocol. Ten μl of beads were tested by PCR with 10 cycles of amplification. The 50 μl of the PCR mixture contained: 10 μl of bead DNA, 15 μl NPM mix (from Illumina Nextera kit), 5 μl of PPC PCR primer, 5 μl of Index Primer 1 (i7), 5 μl of Index Primer 2 (i5), and 10 μl of H₂O. PCR was performed using the following cycle conditions: denaturing the DNA at 72° C. for 3 minutes, then 10-12 cycles of 98° C. for 10 seconds, 63° C. for 30 seconds, and 72° C. for 50 seconds, and a final extension of 72° C. for 5 minutes. The number of cycles was adjusted to obtain about 300 ng of DNA total with four 25 μl reactions. The PCR product may be held at 4° C. for an indefinite amount of time.

The PCR product was cleaned-up using AMPure beads. Beads were allowed to come to RT for 30 minutes before using. Fifty μl of the PCR reaction was transferred to a new Low-Bind Tube and (1.8× volume) 90 μl of AMPure beads was added. The mixture was pipetted well and incubated at RT for 5 minutes. A magnet was used for 3 minutes to collect beads and remove the supernatant. Three hundred μl of freshly prepared 80% ethanol was added to the beads on the magnet, and the ethanol was carefully discarded. The wash was repeated, and then all ethanol was removed. The beads were dried on the magnet rack for 10 minutes. Ten μl EB was added to the beads, mixed well, and incubated for 5 minutes at RT. The eluate was collected, and 1 μl of eluate was used for Qubit and Bioanalyzer.

The library was cloned to verify complexity using the following protocol. One μl of the library was diluted at 1:10. A PCR reaction was performed as described below. Primers that anneal to Illumina adapters were chosen (Tm=52.2° C.). The PCR reaction mixture (total volume: 50 μl) contained the following: 10 μl of 5× GoTaq buffer, 1 μl of 10 mM dNTP, 5 μl of 10 μM primer mix, 0.25 μl of GoTaq polymerase, 1 μl of diluted template DNA, and 32.75 μl of H₂O. PCR was performed using the following cycle conditions: denaturing the DNA at 95° C. for 2 minutes and 20 cycles at the following conditions: 95° C. for 60 seconds, 50° C. for 60 seconds, and 72° C. for 30 seconds with a final extension at 72° C. for 5 minutes. The PCR product may be held at 4° C. for an indefinite amount of time.

The PCR product was ligated with the pGEM® T-Easy vector (Promega) protocol. Five μl of 2× T4 Quick ligase buffer, 1 μl of pGEM® T-Easy vector, 1 μl of T4 ligase, 1 μl of PCR product, and 2 μl of H₂O were combined to a total volume of 10 μl. The product was incubated for 1 hour at RT and 2 μl was used to transform Stellar competent cells. Two hundred μl of 500 μl of cells were plated in SOC media. The next day, 20 colonies are selected for Sanger sequencing using a T7 promoter primer. 60% clones had a full adapter, and 15% had a partial adapter.

viii. Reagents

Protein G Dynabeads for 10 samples were from Invitrogen Dynal, Cat #10003D. Block solution (50 ml) contained 0.25 g BSA dissolved in 50 ml of ddH2O (0.5% BSA, w/v), and was stored at 4° C. for 2 days before use.

Lysis buffer 1 (LB1) (500 ml) contained 25 ml of 1M Hepes-KOH, pH 7.5; 14 ml of 5M NaCl; 1 ml of 0.5 M EDTA, pH 8.0; 50 ml of 100% Glycerol solution; 25 ml of 10% NP-40; and 12.5 ml of 10% Triton X-100. The pH was adjusted to 7.5. The buffer was sterile-filtered, and stored at 4° C. The pH was re-checked immediately prior to use. Lysis buffer 2 (LB2) (1000 ml) contained 10 ml of 1M Tris-HCL, pH 8.0; 40 ml of 5 M NaCl; 2 ml of 0.5 M EDTA, pH 8.0; and 2 ml of 0.5 M EGTA, pH 8.0. The pH was adjusted to 8.0. The buffer was sterile-filtered, and stored at 4° C. The pH was re-checked immediately prior to use.

Sonication buffer (500 ml) contained 25 ml of 1M Hepes-KOH, pH 7.5; 14 ml of 5M NaCl; 1 ml of 0.5 M EDTA, pH 8.0; 50 ml of 10% Triton X-100; 10 ml of 5% Na-deoxycholate; and 5 ml of 10% SDS. The buffer was sterile-filtered, and stored at 4° C. The pH was re-checked immediately prior to use. High-salt sonication buffer (500 ml) contained 25 ml of 1M Hepes-KOH, pH 7.5; 35 ml of 5M NaCl; 1 ml of 0.5 M EDTA, pH 8.0; 50 ml of 10% Triton X-100; 10 ml of 5% Na-deoxycholate; and 5 ml of 10% SDS. The buffer was sterile-filtered, and stored at 4° C. The pH was re-checked immediately prior to use.

LiCl wash buffer (500 ml) contained 10 ml of 1M Tris-HCL, pH 8.0; 1 ml of 0.5M EDTA, pH 8.0; 125 ml of 1M LiCl solution; 25 ml of 10% NP-40; and 50 ml of 5% Na-deoxycholate. The pH was adjusted to 8.0. The buffer was sterile-filtered, and stored at 4° C. The pH was re-checked immediately prior to use.

Elution buffer (500 ml) used to quantify the amount of ChIP DNA contained 25 ml of 1M Tris-HCL, pH 8.0; 10 ml of 0.5M EDTA, pH 8.0; 50 ml of 10% SDS; and 415 ml of ddH₂O. The pH was adjusted to 8.0. The buffer was sterile-filtered, and stored at 4° C. The pH was re-checked immediately prior to use.

ChIA-PET Wash Buffer (50 ml) contained 500 μl of 1M Tris-HCl, pH 8.0 (final 10 mM); 100 μl of 0.5M EDTA, pH 8.0 (final 1 mM); 5 ml of 5M NaCl (final 500 mM); and 44.4 ml of dH₂0.

M. HiChIP

Alternatively to ChIA-PET, HiChIP was used to analyze chromatin interactions and conformation. HiChIP requires fewer cells than ChIA-PET.

i. Cell Crosslinking

Cells were cross-linked as described in the ChIP protocol above. Crosslinked cells were either stored as pellets at −80° C. or used for HiChIP immediately after flash-freezing the cells.

ii. Lysis and Restriction

Fifteen million cross-linked cells were resuspended in 500 μL of ice-cold Hi-C Lysis Buffer and rotated at 4° C. for 30 minutes. For cell amounts greater than 15 million, the pellet was split in half for contact generation and then recombined for sonication. Cells were spun down at 2500 g for 5 minutes, and the supernatant was discarded. The pelleted nuclei were washed once with 500 μL of ice-cold Hi-C Lysis Buffer. The supernatant was removed, and the pellet was resuspended in 100 μL of 0.5% SDS. The resuspension was incubated at 62° C. for 10 minutes, and then 285 μL of H₂O and 50 μL of 10% Triton X-100 were added to quench the SDS. The resuspension was mixed well, and incubated at 37° C. for 15 minutes. Fifty μL of 10× NEB Buffer 2 and 375 U of MboI restriction enzyme (NEB, R0147) was added to the mixture to digest chromatin for 2 hours at 37° C. with rotation. For lower starting material, less restriction enzyme is used: 15 μL was used for 10-15 million cells, 8 μL for 5 million cells, and 4 μL for 1 million cells. Heat (62° C. for 20 minutes) was used to inactivate MboI.

iii. Biotin Incorporation and Proximity Ligation

To fill in the restriction fragment overhangs and mark the DNA ends with biotin, 52 μL of fill-in master mix was reacted by combining 37.5 μL of 0.4 mM biotin-dATP (Thermo 19524016); 1.5 μL of 10 mM dCTP, dGTP, and dTTP; and 10 μL of 5 U/μL DNA Polymerase I, Large (Klenow) Fragment (NEB, M0210). The mixture was incubated at 37° C. for 1 hour with rotation.

948 μL of ligation master mix was added. Ligation Master Mix contained 150 μL of 10× NEB T4 DNA ligase buffer with 10 mM ATP (NEB, B0202); 125 μL of 10% Triton X-100; 3 μL of 50 mg/mL BSA; 10 μL of 400 U/μL T4 DNA Ligase (NEB, M0202); and 660 μL of water. The mixture was incubated at room temperature for 4 hours with rotation. The nuclei were pelleted at 2500 g for 5 minutes, and the supernatant was removed.

iv. Sonication

For sonication, the pellet was brought up to 1000 μL in Nuclear Lysis Buffer. The sample was transferred to a Covaris millitube, and the DNA was sheared using a Covaris® E220Evolution™ with the manufacturer recommended parameters. Each tube (15 million cells) was sonicated for 4 minutes under the following conditions: Fill Level 5; Duty Cycle 5%; PIP 140; and Cycles/Burst 200.

v. Preclearing, Immunoprecipitation, IP Bead Capture, and Washes

The sample was clarified for 15 minutes at 16,100 g at 4° C. The sample was split into 2 tubes of about 400 μL each and 750 μL of ChIP Dilution Buffer was added. For the Smc1a antibody (Bethyl A300-055A), the sample was diluted 1:2 in ChIP Dilution Buffer to achieve an SDS concentration of 0.33%. 60 μL of Protein G beads were washed for every 10 million cells in ChIP Dilution Buffer. Amounts of beads (for preclearing and capture) and antibodies were adjusted linearly for different amounts of cell starting material. Protein G beads were resuspended in 50 μL of Dilution Buffer per tube (100 μL per HiChIP). The sample was rotated at 4° C. for 1 hour. The samples were put on a magnet, and the supernatant was transferred into new tubes. 7.5 μg of antibody was added for every 10 million cells, and the mixture was incubated at 4° C. overnight with rotation. Another 60 μL of Protein G beads for every 10 million cells in ChIP Dilution Buffer was added. Protein G beads were resuspended in 50 μL of Dilution Buffer (100 μL per HiChIP), added to the sample, and rotated at 4° C. for 2 hours. The beads were washed three times each with Low Salt Wash Buffer, High Salt Wash Buffer, and LiCl Wash Buffer. Washing was performed at room temperature on a magnet by adding 500 μL of a wash buffer, swishing the beads back and forth twice by moving the sample relative to the magnet, and then removing the supernatant

vi. ChIP DNA Elution

ChIP sample beads were resuspended in 100 μL of fresh DNA Elution Buffer. The sample beads were incubated at RT for 10 minutes with rotation, followed by 3 minutes at 37° C. with shaking. ChIP samples were placed on a magnet, and the supernatant was removed to a fresh tube. Another 100 μL of DNA Elution Buffer was added to ChIP samples and incubations were repeated. ChIP sample supernatants were removed again and transferred to a new tube. There was about 200 μL of ChIP sample. Ten μL of Proteinase K (20 mg/ml) was added to each sample and incubated at 55° C. for 45 minutes with shaking. The temperature was increased to 67° C., and the samples were incubated for at least 1.5 hours with shaking. The DNA was Zymo-purified (Zymo Research, #D4014) and eluted into 10 μL of water. Post-ChIP DNA was quantified to estimate the amount of Tn5 needed to generate libraries at the correct size distribution. This assumed that contact libraries were generated properly, samples were not over sonicated, and that material was robustly captured on streptavidin beads. SMC1 HiChIP with 10 million cells had an expected yield of post-ChIP DNA from 15 ng-50 ng. For libraries with greater than 150 ng of post-ChIP DNA, materials were set aside and a maximum of 150 ng was taken into the biotin capture step

vii. Biotin Pull-Down and Preparation for Illumina Sequencing

To prepare for biotin pull-down, 5 μL of Streptavidin C-1 beads were washed with Tween Wash Buffer. The beads were resuspended in 10 μL of 2× Biotin Binding Buffer and added to the samples. The beads were incubated at RT for 15 minutes with rotation. The beads were separated on a magnet, and the supernatant was discarded. The beads were washed twice by adding 500 μL of Tween Wash Buffer and incubated at 55° C. for 2 minutes while shaking. The beads were washed in 100 μL of 1× (diluted from 2×) TD Buffer. The beads were resuspended in 25 μL of 2× TD Buffer, 2.5 μL of Tn5 for each 50 ng of post-ChIP DNA, and water to a volume of 50 μL.

The Tn5 had a maximum amount of 4 μL. For example, for 25 ng of DNA transpose, 1.25 μL of Tn5 was added, while for 125 ng of DNA transpose, 4 μL of Tn5 was used. Using the correct amount of Tn5 resulted in proper size distribution. An over-transposed sample had shorter fragments and exhibited lower alignment rates (when the junction was close to a fragment end). An undertransposed sample has fragments that are too large to cluster properly on an Illumina sequencer. The library was amplified in 5 cycles and had enough complexity to be sequenced deeply and achieve proper size distribution regardless of the level of transposition of the library.

The beads were incubated at 55° C. with interval shaking for 10 minutes. Samples were placed on a magnet, and the supernatant was removed. Fifty mM EDTA was added to samples and incubated at 50° C. for 30 minutes. The samples were then quickly placed on a magnet, and the supernatant was removed. The samples were washed twice with 50 mM EDTA at 50° C. for 3 minutes, then were removed quickly from the magnet. Samples were washed twice in Tween Wash Buffer for 2 minutes at 55° C., then were removed quickly from the magnet. The samples were washed with 10 mM Tris-HCl, pH8.0.

viii. PCR and Post-PCR Size Selection

The beads were resuspended in 50 μL of PCR master mix (Nextera XT DNA library preparation kit from Illumina, #15028212 with dual-Index adapters #15055289). PCR was performed using the following program. The cycle number was estimated using one of two methods: (1) A first run of 5 cycles (72° C. for 5 minutes, 98° C. for 1 minute, 98° C. for 15 seconds, 63° C. for 30 seconds, 72° C. for 1 minute) was performed on a regular PCR and then the product was removed from the beads. Then, 0.25× SYBR green was added, and the sample was run on a qPCR. Samples were pulled out at the beginning of exponential amplification; or (2) Reactions are run on a PCR and the cycle number was estimated based on the amount of material from the post-ChIP Qubit (greater than 50 ng is run in 5 cycles, while approximately 50 ng is run in 6 cycles, 25 ng is run in 7 cycles, 12.5 ng is run in 8 cycles, etc.).

Libraries were placed on a magnet and eluted into new tubes. The libraries were purified using a kit form Zymo Research and eluted into 10 μL of water. A two-sided size selection was performed with AMPure XP beads. After PCR, the libraries were placed on a magnet and eluted into new tubes. Then, 25 μL of AMPure XP beads were added, and the supernatant was kept to capture fragments less than 700 bp. The supernatant was transferred to a new tube, and 15 μL of fresh beads were added to capture fragments greater than 300 bp. A final elution was performed from the Ampure XP beads into 10 μL of water. The library quality was verified using a Bioanalyzer.

ix. Buffers

Hi-C Lysis Buffer (10 mL) contained 100 μL of 1M Tris-HCl pH 8.0; 20 μL of 5M NaCl; 200 μL of 10% NP-40; 200 μL of 50× protease inhibitors; and 9.68 mL of water. Nuclear Lysis Buffer (10 mL) contained 500 μL of 1M Tris-HCl pH 7.5; 200 μL of 0.5M EDTA; 1 mL of 10% SDS; 200 μL of 50× Protease Inhibitor; and 8.3 mL of water. ChIP Dilution Buffer (10 mL) contained 10 μL of 10% SDS; 1.1 mL of 10% Triton X-100; 24 μL of 500 mM EDTA; 167 μL of 1M Tris pH 7.5; 334 μL of 5M NaCl; and 8.365 mL of water. Low Salt Wash Buffer (10 mL) contained 100 μL of 10% SDS; 1 mL of 10% Triton X-100; 40 μL of 0.5M EDTA; 200 μL of 1M Tris-HCl pH 7.5; 300 μL of 5M NaCl; and 8.36 mL of water. High Salt Wash Buffer (10 mL) contained 100 μL of 10% SDS; 1 mL of 10% Triton X-100; 40 μL of 0.5M EDTA; 200 μL of 1M Tris-HCl pH 7.5; 1 mL of 5M NaCl; and 7.66 mL of water. LiCl Wash Buffer (10 mL) contained 100 μL of 1M Tris pH 7.5; 500 μL of 5M LiCl; 1 mL of 10% NP-40; 1 mL of 10% Na-deoxycholate; 20 μL of 0.5M EDTA; and 7.38 mL of water.

DNA Elution Buffer (5 mL) contains 250 μL of fresh 1M NaHCO₃; 500 μL of 10% SDS; and 4.25 mL of water. Tween Wash Buffer (50 mL) contained 250 μL of 1M Tris-HCl pH 7.5; 50 μL of 0.5M EDTA; 10 mL of 5M NaCl; 250 μL of 10% Tween-20; and 39.45 mL of water. 2× Biotin Binding Buffer (10 mL) contained 100 μL 1M Tris-HCl pH 7.5; 20 μL of 0.5M; 4 mL of 5M NaCl; and 5.88 mL of water. 2× TD Buffer (1 mL) contained 20 μL of 1M Tris-HCl pH 7.5; 10 μL of 1M MgCl₂; 200 μL of 100% Dimethylformamide; and 770 μL of water.

N. Drug Dilutions for Administration to Hepatocytes

Prior to compound treatment of hepatocytes, 100 mM stock drugs in DMSO were diluted to 10 mM by mixing 0.1 mM of the stock drug in DMSO with 0.9 ml of DMSO to a final volume of 1.0 ml. Five μl of the diluted drug was added to each well, and 0.5 ml of media was added per well of drug. Each drug was analyzed in triplicate. Dilution to 1000× was performed by adding 5 μl of drug into 45 μl of media, and the 50 μl being added to 450 μl of media on cells.

Bioactive compounds were also administered to hepatocytes. To obtain 1000× stock of the bioactive compounds in 1 ml DMSO, 0.1 ml of 10,000× stock was combined with 0.9 ml DMSO.

O. siRNA Knockdown

Primary human hepatocytes were reverse transfected with siRNA with 6 pmol siRNA using RNAiMAX Reagent (ThermoFisher Cat #13778030) in 24 well format, 1 μl per well. The following morning, the medium was removed and replaced with modified maintenance medium for an additional 24 hours. The entire treatment lasted 48 hours, at which point the medium was removed and replaced with RLT Buffer for RNA extraction (Qiagen RNeasy 96 QIAcube HT Kit Cat #74171). Cells were processed for qRT-PCR analysis and then levels of target mRNA were measured.

siRNAs were obtained from Dharmacon and were a pool of four siRNA duplex all designed to target distinct sites within the specific gene of interest (“SMARTpool”).

P. Mice Studies

A group of 6 mice (C57BL/6J strain), 3 male and 3 female, are administered with a candidate compound once daily via oral gavage for four consecutive days. Mice were sacrificed 4 hours post-last dose on the fourth day. Organs including liver, spleen, kidney, adipose, plasma are collected. Mouse liver tissues were pulverized in liquid nitrogen and aliquoted into small microtubes. TRIzol (Invitrogen Cat #15596026) was added to the tubes to facilitate cell lysis from tissue samples. The TRIzol solution containing the disrupted tissue was then centrifuged and the supernatant phase is collected. Total RNA was extracted from the supernatant using Qiagen RNA Extraction Kit (Qiagen Cat #74182) and the target mRNA levels were analyzed using qRT-PCR.

### Example 2. RNA-seq Study for Stimulated Hepatocytes

To identify small molecules that modulate target gene expression, primary human hepatocytes were prepared as a monoculture, and at least one small molecule compound was applied to the cells.

RNA-seq was performed to determine the effects of the compounds on the expression of the target genes in hepatocytes. Fold change was calculated by dividing the level of expression in the cell system that had been perturbed by the level of expression in an unperturbed system. Changes in expression having a p-value ≤0.05 were considered significant.

Compounds used to perturb the signaling centers of hepatocytes include at least one compound listed in Table 2. In the table, compounds are listed with their ID, target, pathway, and pharmaceutical action. Most compounds chosen as perturbation signals are known in the art to modulate at least one canonical cellular pathway. Some compounds were selected from compounds that failed in Phase III clinical evaluation due to lack of efficacy.

TABLE 2
Compounds used in RNA-seq

| ID | Compound Name | Target | Pathway | Action |
|---|---|---|---|---|
| 1 | Simvastatin | HMG-CoA | Metabolic | Inhibitor |
| | | reductase | | |
| 2 | Adapin (doxepin) | H₁ histamine, α- | Histamine receptor signaling | Antagonist |
| | | adrenoreceptors | | |
| 4 | Danazol | ER, AR, | Estrogen signaling | Agonist |
| | | Progesteron | | |
| | | receptor | | |
| 5 | Nefazodone | HTR2A | Calcium signaling | Antagonist |
| 6 | Rosiglitazone maleate | PPARg | PPAR signaling | Agonist |
| 7 | Sulpiride | D₂ dopamine | cAMP signaling | Antagonist |
| 8 | Captopril | MMP2 | Estrogen signaling | Inhibitor |
| 9 | atenolol | ADRB1 | Adrenergic signaling | Antagonist |
| 10 | Ranitidine | H₂ histamine | Histamine receptor signaling | Antagonist |
| | | receptor | | |
| 11 | Metformin | AMPK | Insulin & AMPK signaling | Activator |
| 12 | imatinib | RTK, Bcr-Abl | PDGFR, ABL signaling | Inhibitor |
| 13 | Papaverine | phosphodiesterase | AMPK signaling | Inhibitor |
| 14 | Amiodarone | Adrenergic | Adrenergic signaling | Antagonist |
| | | receptor β, CYP | | |
| 15 | Nitrofurantoin | pyruvate- | antibiotic | Activator |
| | | flavodoxin | | |
| | | oxidoreductase | | |
| 16 | prednisone | GR | GR signaling | Agonist |
| 17 | Penicillamine(D−) | copper | copper chelation | Chelator |
| 18 | Disopyramide | SCN5A | Adrenergic signaling | Inhibitor |
| 19 | Rifampicin | PXR | PXR | Inhibitor |
| 20 | Benzbromarone | xanthine oxidase, | uric acid formation | Inhibitor |
| | | CYP2C9 | | |
| 21 | isoniazid | CYP2C19, | unknown | Inhibitor |
| | | CYP3A4 | | |
| 22 | Acetaminophen | COX1/2 | COX | Inhibitor |
| | (paracetamol) | | | |
| 23 | Ritonavir | CYP3A4, Pol | HIV Transcription | Inhibitor |
| | | polyprotein | | |
| 24 | SGI-1776 | PIM | JAK/STAT signaling | Inhibitor |
| 25 | Valproate | HDAC9, glucuronyl | unknown | Inhibitor |
| | | transferase, | | |
| | | epoxide hydrolase | | |
| 26 | Ibuprofen | COX, PTGS2 | COX | Inhibitor |
| 27 | Propylthiouracil | thyroperoxidase | Thyroid hormone synthesis | Inhibitor |
| 28 | rapamycin | mTOR | mTOR signaling | Inhibitor |
| 29 | BIO | GSK-3 | WNT, TGF beta signaling | Inhibitor |
| 30 | ATRA | RXRb, RXRg, | RAR signaling | Agonist |
| | | RARg | | |
| 31 | Xav939 | tankyrase | WNT & PARP pathway | Inhibitor |
| 32 | bms189453 | RARB | Nuclear Receptor transcription | Agonist |
| 33 | dorsomorphin | ALK | TGF beta signaling | Inhibitor |
| 34 | BMP2 | BMPR1A | TGF beta signaling | Agonist |
| 35 | BMS777607 | Met | Ras signaling | Inhibitor |
| 36 | bms833923 | SMO | Hedgehog signaling | Antagonist |
| 37 | dmPGE2 | EPR, PGDH | EP receptor signaling | Agonist |
| 38 | MK-0752 | y-secretase | NOTCH signaling | Inhibitor |
| 39 | N-Acetylpurinomycin | SnoN, SKI, SKIL | TGF beta signaling | Modulator |
| 40 | LY 364947 | TGF-β RI, TGFR- | TGF beta signaling | Inhibitor |
| | | I, TβR-I, ALK-5 | | |
| 41 | Enzastaurin | PKC | Epigenetics; TGF-beta/Smad | Inhibitor |
| 42 | DMXAA | Unclear | Tumor necrosis | Inhibitor |
| 43 | BSI-201 | PARP | Cell Cycle/DNA Damage; | Inhibitor |
| | | | Epigenetics | |
| 44 | Darapladib | Phospholipase | Others | Inhibitor |
| 45 | Selumetinib | MEK | MAPK/ERK Pathway | Inhibitor |
| 46 | Peramivir (trihydrate) | Influenza Virus | Anti-infection | Inhibitor |
| 47 | Palifosfamide | DNA | Cell Cycle/DNA Damage | |
| | | alkylator/crosslinker | | |
| 48 | Evacetrapib | CETP | Others | Inhibitor |
| 49 | Cediranib | VEGFR | Protein Tyrosine Kinase/RTK | Inhibitor |
| 50 | R788 (fostamatinib, | Syk | Protein Tyrosine Kinase/RTK | Inhibitor |
| | disodium hexahydrate) | | | |
| 51 | Torcetrapib | CETP | Others | Inhibitor |
| 52 | Tivozanib | VEGFR | Protein Tyrosine Kinase/RTK | Inhibitor |
| 53 | 17-AAG | HSP | Cell Cycle/DNA Damage | Inhibitor |
| | (Tanespimycin) | | Metabolic Enzyme/Protease | |
| 54 | Zibotentan | Endothelin | GPCR/G protein | Antagonist |
| | | Receptor | | |
| 55 | Semagacestat | γ-secretase | Neuronal Signaling Stem | Inhibitor |
| | | | Cells/Wnt | |
| 56 | Dalcetrapib | CETP | Others | Inhibitor |
| 57 | Latrepirdine | AMPK | Epigenetics; PI3K/Akt/mTOR | Activator |
| | (dihydrochloride) | | | |
| 58 | CMX001 | CMV | Anti-infection | NA |
| | (Brincidofovir) | | | |
| 59 | Vicriviroc (maleate) | CCR | GPCR/G protein; | Antagonist |
| | | | Immunology/Inflammation | |
| 60 | Temsirolimus | mTOR | PI3K/Akt/mTOR | Inhibitor |
| 61 | Preladenant | Adenosine | GPCR/G protein | Antagonist |
| | | Receptor | | |
| 62 | EVP-6124 | nAChR | Membrane Transporter/Ion | Activator |
| | (hydrochloride) | | Channel | |
| | (encenicline) | | | |
| 63 | Bitopertin | GlyT1 | Membrane Transporter/Ion | Inhibitor |
| | | | Channel | |
| 64 | Latrepirdine | AMPK | Epigenetics; PI3K/Akt/mTOR | Inhibitor |
| 65 | Vanoxerine | Dopamine | Neuronal Signaling | Inhibitor |
| | (dihydrochloride) | Reuptake Inhibitor | | |
| 66 | CO-1686 (Rociletinib) | EGFR | JAK/STAT Signaling Protein | Inhibitor |
| | | | Tyrosine Kinase/RTK | |
| 67 | Laropiprant | Prostaglandin | GPCR/G protein | Antagonist |
| | (tredaptive) | Receptor | | |
| 68 | Bardoxolone | Keap1-Nrf2 | NF-ϰB | Activator |
| 69 | VX-661 (tezacaptor) | CFTR | Membrane transporter/ion | Corrector |
| | | | channel | |
| 70 | INNO-206 | Topoisomerase | Cell Cycle/DNA Damage | NA |
| | (aldoxorubicin) | | | |
| 71 | LY404039 | mGluR | GPCR/G protein | Inhibitor |
| | (pomaglumetad | | | |
| | methionil (mGlu2/3)) | | | |
| 72 | Perifosine (KRX- | AKT | PI3K/AKT | Inhibitor |
| | 0401) | | | |
| 73 | Cabozantinib (XL184, | VEGFR2, MET, | MET | Inhibitor |
| | BMS-907351) | Ret, Kit, Flt-1/3/4, | | |
| | | Tie2, and AXL | | |
| 74 | Dacomitinib | EGFR, ErbB2, | AKT/ERK, HER | Inhibitor |
| | (PF299804, PF299) | ErbB4 | | |
| 75 | Pacritinib (SB1518) | FLT3, JAK2, | JAK-STAT | Inhibitor |
| | | TYK2, JAK3, | | |
| | | JAK1 | | |
| 76 | TH-302 | hypoxic regions | Unclear | NA |
| | (Evofosfamide) | | | |
| 77 | α-PHP | Unclear | Unclear | NA |
| 78 | LY 2140023 | mGlu₂ & mGlu₃ | Gαi/o protein-dependent | Activator |
| | (Pomaglumetad | | | |
| | methionil-LY404039) | | | |
| 79 | TP-434 (Eravacycline) | Antibiotic | Tetracycline-specific efflux | Inhibitor |
| | | resistance | | |
| | | mechanisms | | |
| 80 | TC-5214 (S-(+)- | Nicotinic | Base excision repair and | Antagonist |
| | MecaMylaMine | acetylcholine | homologous recombination | |
| | Hydrochloride) | receptors | repair | |
| 81 | Rolofylline (KW- | A1 adenosine | Unclear | Antagonist |
| | 3902) | receptor | | |
| 82 | Amigal | a-galactosidase | Stress signaling | Inhibitor |
| | (Deoxygalactonojirimycin | | | |
| | hydrochloride) | | | |
| 83 | NOV-002 (oxidized L- | gamma-glutamyl- | Glutathione pathway | NA |
| | Glutathione) | transpeptidase | | |
| | | (GGT) | | |
| 84 | bms-986094 (inx-189) | NS5B | Unclear | Inhibitor |
| 85 | TC-5214 (R- | Nicotinic receptors | Base excision repair and | Antagonist |
| | Mecamylamine | | homologous recombination | |
| | hydrochloride) | | repair | |
| 86 | Ganaxolone | GBAA receptors | Unclear | Modulator |
| 87 | Irinotecan | DNA Topo I | Unclear | Inhibitor |
| | Hydrochloride | | | |
| | Trihydrate | | | |
| 88 | TFP | D2R, Calmodulin | Calmodulin | Inhibitor |
| 89 | Perphenazine | D2R, Calmodulin | Calmodulin | Inhibitor |
| 90 | A3-HCl | CKI, CKII, PKC, | WNT, Hedgehog, PKC, PKA | Inhibitor |
| | | PKA | | |
| 91 | FICZ | Aryl hydrocarbon | Aryl hydrocarbon receptor | Agonist |
| | | receptor | | |
| 92 | Pifithrin-a | p53 | p53 | Inhibitor |
| 93 | Deferoxamine | HIF | Hypoxia activated | Inhibitor |
| | mesylate | | | |
| 94 | Insulin | InsR | IGF-1R/InsR | Activator |
| 95 | Phorbol 12,13- | PKC | PKC | Activator |
| | dibutyrate | | | |
| 96 | RU 28318 | MR | Mineralcorticoid receptor | Antagonist |
| 97 | Bryostatin1 | PKC | PKC | Activator |
| 98 | DY 268 | FXR | FXR | Antagonist |
| 99 | GW 7647 | PPARα | PPAR | Agonist |
| 100 | CI-4AS-1 | AR | Androgen receptor | Agonist |
| 101 | T0901317 | LXR | LXR | Agonist |
| 102 | BMP2 | BMPR1A | TGF-B | Activator |
| 103 | 22S- | LXR | LXR | Inhibitor |
| | Hydroxycholesterol | | | |
| 104 | CALP1 | Calmodulin | Calmodulin | Activator |
| 105 | CALP3 | Calmodulin | Calmodulin | Activator |
| 106 | Forskolin | Adenylyl cyclase | cAMP related | Activator |
| 107 | Dexamethasone | GR | Glucocorticoid receptor | Activator |
| 108 | IFN-y | IFNGR1/IFNGR2 | JAK/STAT | Activator |
| 109 | TGF-b | TGF-beta Receptor | TGF-B | Activator |
| 110 | TNF-α | TNF-R1/TNF-R2 | NF-kB, MAPK, Apoptosis | Activator |
| 111 | PDGF | Pan-PDGFR | PDGFR | Activator |
| 112 | IGF-1 | IGF-1R | IGF-1R/InsR | Activator |
| 113 | FGF | FGFR | FGFR | Activator |
| 114 | EGF | Pan-ErbB | EGFR | Activator |
| 115 | HGF/SF | c-Met | c-MET | Activator |
| 116 | TCS 359 | FLT3 | Protein Tyrosine Kinase/RTK | Inhibitor |
| 117 | Cobalt chloride | HIF1 | Hypoxia activated | Inducer |
| 118 | CH223191 | AhR | Aryl hydrocarbon receptor | Antagonist |
| 119 | Echinomycin | HIF | Hypoxia activated | Inhibitor |
| 120 | PAF C-16 | MEK | MAPK | Activator |
| 121 | Bexarotene | RXR | RXR | Agonist |
| 122 | CD 2665 | RAR | RAR | Antagonist |
| 123 | Pifithrin-μ | p53 | p53 | Inhibitor |
| 124 | EB1089 | VDR | Vitamin D Receptor | Agonist |
| 125 | BMP4 | TGF-beta | TGF-B | Activator |
| 126 | IWP-2 | Wnt | WNT | Inhibitor |
| 127 | RITA (NSC 652287) | p53 | p53 | Inhibitor |
| 128 | Calcitriol | VDR | Vitamin D Receptor | Agonist |
| 129 | ACEA | CB1 | Cannabinoid receptor | Agonist |
| 130 | Rimonabant | CB1 | Cannabinoid receptor | Antagonist |
| 131 | Otenabant | CB1 | Cannabinoid receptor | Antagonist |
| 132 | DLPC | LRH-1/NR5A2 | LHR-1 | Agonist |
| 133 | LRH-1 antagonist | LRH-1/NR5A3 | LHR-1 | Antagonist |
| 134 | Wnt3a | FRIZZLED | WNT | Activator |
| 135 | Activin | TGF-beta | TGF-B | Activator |
| 136 | Nodal | TGF-beta | TGF-B | Activator |
| 137 | Anti mullerian | TGF-beta | TGF-B | Activator |
| | hormone | | | |
| 138 | GDF2 (BMP9) | TGF-beta | TGF-B | Activator |
| 139 | GDF10 (BMP3b) | TGF-beta | TGF-B | Activator |
| 140 | Oxoglaucine | PI3K/Akt | PI3K/AKT | Activator |
| 141 | BMS 195614 | RAR | RAR | Antagonist |
| 142 | LDN193189 | ALK2/3 | TGF-B | Inhibitor |
| 143 | Varenicline Tartrate | AchR | Acetylcholine receptor | Agonist |
| 144 | Histamine | Histamine receptor | Histamine receptor | Activator |
| 145 | Chloroquine | ATM/ATR | ATM/ATR | Activator |
| | phosphate | | | |
| 146 | LJI308 | RSK1/2/3 | S6K | Inhibitor |
| 147 | GSK621 | AMPK | AMPK | Activator |
| 148 | STA-21 | STAT3 | JAK/STAT | Inhibitor |
| 149 | SMI-4a | Pim1 | PIM | Inhibitor |
| 150 | AMG 337 | c-Met | c-MET | Inhibitor |
| 151 | Wnt agonist 1 | Wnt | WNT | Activator |
| 152 | PRI-724 | Wnt | WNT | Inhibitor |
| 153 | ABT-263 | Pan-Bcl-2 | BCL2 | Inhibitor |
| 154 | Axitinib | Pan-VEGFR | VEGFR | Inhibitor |
| 155 | Afatinib | Pan-ErbB | EGFR | Inhibitor |
| 156 | Bosutinib | Src | Src | Inhibitor |
| 157 | Dasatinib | Bcr-Abl | ABL | Inhibitor |
| 158 | Masitinib | c-Kit | c-KIT | Inhibitor |
| 159 | Crizotinib | c-Met | c-MET | Inhibitor |
| 160 | PHA-665752 | c-Met | c-MET | Inhibitor |
| 161 | GSK1904529A | IGF-lR/InsR | IGF-lR/InsR | Inhibitor |
| 162 | GDC-0879 | Raf | MAPK | Inhibitor |
| 163 | LY294002 | Pan-PI3K | PI3K/AKT | Inhibitor |
| 164 | OSU-03012 | PDK-1 | PDK-1 | Inhibitor |
| 165 | JNJ-38877605 | c-Met | c-MET | Inhibitor |
| 166 | BMS-754807 | IGF-1R/InsR | IGF-lR/InsR | Inhibitor |
| 167 | TGX-221 | p110b | PI3K/AKT | Inhibitor |
| 168 | Regorafenib | Pan-VEGFR | VEGFR | Inhibitor |
| 169 | Thalidomide | AR | NF-kB | Antagonist |
| 170 | Amuvatinib | PDGFRA | PDGFR | Inhibitor |
| 171 | Etomidate | GABA | GABAergic receptor | Inhibitor |
| 172 | Glimepiride | Potassium channel | Potassium channel | Inhibitor |
| 173 | Omeprazole | Proton pump | Proton pump | Agonist |
| 174 | Tipifarnib | Ras | RAS | Inhibitor |
| 175 | SP600125 | Jnk | MAPK | Inhibitor |
| 176 | Quizartinib | FLT3 | FLT3 | Inhibitor |
| 177 | CP-673451 | Pan-PDGFR | PDGFR | Inhibitor |
| 178 | Pomalidomide | TNF-α | NF-kB | Inhibitor |
| 179 | KU-60019 | ATM kinase | DNA Damage | Inhibitor |
| 180 | BIRB 796 | p38 | MAPK | Inhibitor |
| 181 | RO4929097 | Gamma-secretase | NOTCH | Inhibitor |
| 182 | Hydrocortisone | GR | Glucocorticoid receptor | Agonist |
| 183 | AICAR | AMPK | AMPK | Activator |
| 184 | Amlodipine Besylate | Calcium channel | Calcium channel | Inhibitor |
| 185 | DPH | Bcr-Abl | ABL | Activator |
| 186 | Taladegib | Hedgehog/Smoothened | Hedgehog/Smoothened | Inhibitor |
| 187 | AZD1480 | JAK2 | JAK/STAT | Inhibitor |
| 188 | AST-1306 | Pan-ErbB | EGFR | Inhibitor |
| 189 | AZD8931 | Pan-ErbB | EGFR | Inhibitor |
| 190 | Momelotinib | Pan-Jak | JAK/STAT | Inhibitor |
| 191 | Cryptotanshinone | STAT3 | JAK/STAT | Inhibitor |
| 192 | Bethanechol chloride | AchR | Acetylcholine receptor | Activator |
| 193 | Clozapine | 5-HT | 5-HT | Antagonist |
| 194 | Dopamine | Dopamine | Dopamine receptor | Agonist |
| 195 | Phenformin | AMPK | AMPK | Activator |
| 196 | Mifepristone | GR | Glucocorticoid receptor | Antagonist |
| 197 | GW3965 | LXR | LXR | Agonist |
| 198 | WYE-125132 (WYE- | mTOR | mTOR | Inhibitor |
| | 132) | | | |
| 199 | Crenolanib | Pan-PDGFR | PDGFR | Inhibitor |
| 200 | PF-04691502 | Pan-Akt | PI3K/AKT | Inhibitor |
| 201 | GW4064 | FXR | FXR | Agonist |
| 202 | Sotrastaurin | PKC | PKC | Inhibitor |
| 203 | Ipatasertib | Pan-Akt | PI3K/AKT | Inhibitor |
| 204 | ARN-509 | AR | Androgen receptor | Inhibitor |
| 205 | T0070907 | PPARg | PPAR | Antagonist |
| 206 | GO6983 | PKC | PKC | Inhibitor |
| 207 | Epinephrine | Adrenergic | Adrenergic receptor | Agonist |
| 208 | Eletriptan | 5-HT | 5-HT | Agonist |
| 209 | Trifluoperazine | Dopamine | Dopamine receptor | Inhibitor |
| 210 | Fexofenadine | Histamine | Histamine receptor | Inhibitor |
| 211 | Deoxycorticosterone | MR | Mineralcorticoid receptor | Agonist |
| 212 | Tamibarotene | RAR | RAR | Agonist |
| 213 | Leucine | mTOR | mTOR | Activator |
| 214 | Glycopyrrolate | AchR | Acetylcholine receptor | Antagonist |
| 215 | Tiagabine | GABA | GABAergic receptor | Inhibitor |
| 216 | Fluoxymesterone | AR | Androgen receptor | Agonist |
| 217 | Tamsulosin | Adrenergic | Adrenergic receptor | Antagonist |
| | hydrochloride | | | |
| 218 | Ceritinib | ALK | ALK | Inhibitor |
| 219 | GSK2334470 | PDK-1 | PDK-1 | Inhibitor |
| 220 | AZD1208 | Pan-PIM | PIM | Inhibitor |
| 221 | CGK733 | ATM/ATR | DNA Damage | Inhibitor |
| 222 | LDN-212854 | Pan-TGFB | TGF-B | Inhibitor |
| 223 | GZD824 Dimesylate | Bcr-Abl | ABL | Inhibitor |
| 224 | AZD2858 | Pan-GSK-3 | GSK-3 | Inhibitor |
| 225 | FRAX597 | PAK | PAK | Inhibitor |
| 226 | SC75741 | NF-kB | NF-kB | Inhibitor |
| 227 | SH-4-54 | Pan-STAT | JAK/STAT | Inhibitor |
| 228 | HS-173 | p110a | PI3K/AKT | Inhibitor |
| 229 | K02288 | Pan-TGFB | TGF-B | Inhibitor |
| 230 | EW-7197 | Pan-TGFB | TGF-B | Inhibitor |
| 231 | Decernotinib | Pan-Jak | JAK/STAT | Inhibitor |
| 232 | MI-773 | p53 | p53 | Inhibitor |
| 233 | PND-1186 | FAK | FAK | Activator |
| 234 | Kartogenin | SMAD4/5 | TGF-B | Activator |
| 235 | Picropodophyllin | IGF-1R | IGF-1R/InsR | Inhibitor |
| 236 | AZD6738 | ATR | ATM/ATR | Inhibitor |
| 237 | Smoothened Agonist | Hedgehog/Smoothened | Hedgehog/Smoothened | Agonist |
| 238 | Erlotinib | EGFR/ErbB1 | EGFR | Inhibitor |
| 239 | MHY1485 | mTOR | mTOR | Activator |
| 240 | SC79 | Pan-Akt | PI3K/AKT | Activator |
| 241 | meBIO | AhR | Aryl hydrocarbon receptor | Agonist |
| 242 | Huperzine | AchE | Acetylcholine receptor | Inhibitor |
| 243 | BGJ398 | Pan-FGFR | FGFR | Inhibitor |
| 244 | Netarsudil | ROCK | ROCK | Inhibitor |
| 245 | Acetycholine | AchR | Acetylcholine receptor | Agonist |
| 246 | Purmorphamine | Hedgehog/Smoothened | Hedgehog/Smoothened | Agonist |
| 247 | LY2584702 | p70 S6K | S6K | Inhibitor |
| 248 | Dorsomorphin | AMPK | AMPK | Inhibitor |
| 249 | Glasdegib (PF- | Hedgehog/Smoothened | Hedgehog/Smoothened | Inhibitor |
| | 04449913) | | | |
| 250 | LDN193189 | Pan-TGFB | TGF-B | Inhibitor |
| 251 | Oligomycin A | ATPase | ATP channel | Inhibitor |
| 252 | BAY 87-2243 | HIF1 | Hypoxia activated | Inhibitor |
| 253 | SIS3 | SMAD3 | TGF-B | Inhibitor |
| 254 | BDA-366 | Bcl-2 | BCL2 | Antagonist |
| 255 | XMU-MP-1 | MST1/2 | Hippo | Inhibitor |
| 256 | Semaxanib | Pan-VEGFR | VEGFR | Inhibitor |
| 257 | BAM7 | Bcl-2 | BCL2 | Activator |
| 258 | GDC-0994 | Erk | MAPK | Inhibitor |
| 259 | SKL2001 | Wnt | WNT | Agonist |
| 260 | Merestinib | c-Met | c-MET | Inhibitor |
| 261 | APS-2-79 | MEK | MAPK | Antagonist |
| 262 | NSC228155 | Pan-ErbB | EGFR | Activator |
| 263 | 740 Y-P | Pan-PI3K | PI3K/AKT | Activator |
| 264 | b-Estradiol | ER | ER | Activator |
| 265 | Glucose | GLUTs | metabolic/glycolysis | Activator |
| 266 | Transferrin | Transferrin | Iron transport | Activator |
| | | Receptor | | |
| 267 | AM 580 | RAR | RAR | Activator |

### Example 3. Identification of Treatments for Diseases, Disorders, and Conditions

Analysis of RNA-seq data revealed a number of compounds that caused significant changes in the expression of the target genes. Significance was defined as an FPKM ≥1, a log 2(fold change) ≥0.5, and a q-value of ≤0.05 for all targets except for CPOX. RNA-seq results for compounds that significantly modulated at least one selected target gene are shown in Tables 3-12.

Table 3 provides the log 2 fold change for compounds that were observed to significantly decrease expression of FN1, encoding fibronectin, which is associated with fibronectin glomerulopathy.

TABLE 3
RNA-seq results for FN1

| Compounds | Fold Change (Log2) |
|---|---|
| Smoothened Agonist | −1.95 |
| Crizotinib | −1.45 |
| BGJ398 | −1.36 |
| AZD2858 | −1.23 |
| Amlodipine Besylate | −1.22 |
| PHA-665752 | −1.14 |
| OSU-03012 | −1.03 |
| bms-986094 (inx-189) | −1.00 |
| Afatinib | −0.98 |
| LDN193189 | −0.87 |
| Sotrastaurin | −0.81 |
| SKL2001 | −0.81 |
| Tivozanib | −0.8 |
| Cediranib | −0.79 |
| Calcitriol | −0.79 |
| Rimonabant | −0.76 |

Table 4 provides the log 2 fold change for compounds that were observed to significantly increase expression of CPOX, encoding coproporphyrinogen oxidase, which is associated with hereditary coproporphyria. Significance was defined as an FPKM ≥0.5 a log 2(fold change) ≥0.3, and a q-value of ≤0.05.

TABLE 4
RNA-seq results for CPOX

| Compounds | Fold change (Log2) |
|---|---|
| Thalidomide | 0.3 |
| Glycopyrrolate | 0.31 |
| MK-0752 | 0.34 |
| Bosutinib | 0.34 |
| Nefazodone | 0.35 |
| Corticosterone | 0.36 |
| Deferoxamine mesylate | 0.37 |
| GZD824 Dimesylate | 0.37 |
| XMU-MP-1 | 0.37 |
| prednisone | 0.44 |
| FICZ | 0.44 |
| SKL2001 | 0.45 |
| Cobalt chloride | 0.49 |
| 17-AAG (Tanespimycin) | 0.53 |

Table 5 provides the log 2 fold change for compounds that were observed to significantly increase expression of SERPINC1, encoding antithrombin, which is associated with SERPINC1 deficiency.

TABLE 5
RNA-seq results for SERPINC1

| Compounds | Fold Change (Log2) |
|---|---|
| CP-673451 | 2.53 |
| Echinomycin | 2.29 |
| Pacritinib (SB1518) | 2.15 |
| Amuvatinib | 1.33 |
| Crenolanib | 1.31 |
| INNO-206 (aldoxorubicin) | 1.00 |
| Momelotinib | 0.93 |
| Thalidomide | 0.88 |
| Pifithrin-u | 0.79 |

Table 6 provides the log 2 fold change for compounds that were observed to significantly increase expression of JAG1 and/or NOTCH2, encoding jagged 1 and Notch 2 respectively, which are associated with Alagille Syndrome. The bolded compounds represent those that significantly modulate both JAG1 and NOTCH2.

TABLE 6
RNA-seq results for JAG1 and NOTCH2

Fold Change (Log2)

| Compounds | JAG1 | NOTCH2 |
|---|---|---|
| LDN193189 | 2.07 | 0 |
| LDN-212854 | 1.47 | 0 |
| Thalidomide | 1.30 | −0.68 |
| Phenformin | 1.14 | 0 |
| Enzastaurin | 1.03 | 0 |
| GDF2 (BMP9) | 1.03 | 0 |
| BMP2 | 0.99 | 0 |
| INNO-206 (aldoxorubicin) | 0.84 | 0 |
| Merestinib | 0.76 | 0.62 |
| Amuvatinib | 0.74 | −0.79 |
| Torcetrapib | 0.68 | 0.76 |
| BMP4 | 0.63 | 0 |
| BAY 87-2243 | 0.61 | 0 |
| Zibotentan | 0 | 0.94 |
| 740 Y-P | 0 | 0.66 |

As shown above, LDN193189, LDN-212854, thalidomide, phenformin, enzastaurin, GDF2 (BMP9), BMP2, amuvatinib, BMP4, and BAY 87-2243, and INNO-206 (aldoxorubicin) were observed to significantly modulate only JAG1; and zibotentan and 740 Y-P were observed to significantly modulate only NOTCH2. Merestinib and torcetrapib were observed to significantly modulate both JAG1 and NOTCH2.

Table 7 provides the log 2 fold change for compounds that were observed to significantly increase expression of SLC37A4, encoding glucose-6-phosphate translocase (G6PT), which is associated with Glycogen Storage disease 1b.

TABLE 7
RNA-seq results for SLC37A4

| Compounds | Fold Change (Log2) |
|---|---|
| Echinomycin | 1.59 |
| prednisone | 1.18 |
| CP-673451 | 1.17 |
| Cobalt chloride | 1.09 |
| Amuvatinib | 0.97 |
| Pacritinib (SB1518) | 0.93 |
| R788 (fostamatinib disodium | 0.74 |
| hexahydrate) | |
| GZD824 Dimesylate | 0.74 |
| Corticosterone | 0.73 |
| Dexamethasone | 0.66 |
| TNF-a | 0.64 |
| Thalidomide | 0.64 |
| IGF-1 | 0.60 |

Table 8 provides the log 2 fold change for compounds that were observed to significantly increase expression of HMBS, encoding hydroxymethylbilane synthase, which is associated with acute intermittent porphyria.

TABLE 8
RNA-seq results for HMBS

| Compound | Fold Change (Log2) |
|---|---|
| Sotrastaurin | 0.78 |

Table 9 provides the log 2 fold change for compounds that were observed to significantly decrease expression of LECT2, encoding leukocyte cell derived chemotaxin 2, which is associated with LECT2 amyloidosis.

TABLE 9
RNA-seq results for LECT2

| Compounds | Fold Change (Log2) |
|---|---|
| Calcitriol | −1.82 |
| 17-AAG (Tanespimycin) | −1.00 |
| Ritonavir | −1.00 |
| TFP | −0.91 |
| b-Estradiol | −0.90 |
| Rifampicin | −0.88 |
| Torcetrapib | −0.86 |
| Zibotentan | −0.83 |
| Rimonabant | −0.81 |
| OSU-03012 | −0.77 |
| Afatinib | −0.75 |
| NSC228155 | −0.74 |
| Glucose | −0.73 |
| APS-2-79 | −0.68 |
| Phorbol 1213-dibutyrate | −0.65 |
| prednisone | −0.65 |
| 740 Y-P | −0.62 |
| Amlodipine Besylate | −0.55 |
| Darapladib | −0.55 |

Table 10 provides the log 2 fold change for compounds that were observed to significantly decrease expression of APOL1, encoding apolipoprotein L1, which is associated with APOL1-associated glomerular disease.

TABLE 10
RNA-seq results for APOL1

| Compounds | Fold Change (Log2) |
|---|---|
| Nitrofurantoin | −0.60 |
| Crizotinib | −0.56 |

Table 11 provides the log 2 fold change for compounds that were observed to significantly increase expression of UGT1A1, encoding UDP glucuronosyltransferase family 1 member A1, which is associated with Gilbert Syndrome and Criggler Najjar, type II.

TABLE 11
RNA-seq results for UGT1A1

| Compounds | Fold Change (Log2) |
|---|---|
| FICZ | 2.62 |
| Kartogenin | 2.24 |
| meBIO | 1.98 |
| CP-673451 | 1.82 |
| BAM7 | 1.66 |
| EW-7197 | 1.66 |
| Pacritinib (SB1518) | 1.58 |
| Pifithrin-a | 1.44 |
| LY294002 | 1.36 |
| BMS-754807 | 1.33 |
| Bexarotene | 1.29 |
| Crizotinib | 1.18 |
| ARN-509 | 1.16 |
| Echinomycin | 1.10 |
| JNJ-38877605 | 1.09 |
| Omeprazole | 1.09 |
| RO4929097 | 1.06 |
| Momelotinib | 1.05 |
| BIRB 796 | 1.04 |
| AZD6738 | 1.03 |
| Semagacestat | 1.01 |
| Glimepiride | 0.96 |
| AZD1480 | 0.93 |
| Cryptotanshinone | 0.93 |
| GW4064 | 0.92 |
| LRH-1 antagonist | 0.91 |
| PND-1186 | 0.90 |
| Crenolanib | 0.89 |
| EB1089 | 0.88 |
| Sotrastaurin | 0.88 |
| Corticosterone | 0.86 |
| GZD824 Dimesylate | 0.86 |
| Netarsudil | 0.85 |
| R788 (fostamatinib disodium | 0.85 |
| hexahydrate) | |
| Oxoglaucine | 0.83 |
| Evacetrapib | 0.78 |
| LY2584702 | 0.76 |
| Merestinib | 0.76 |
| CI-4AS-1 | 0.74 |
| Dasatinib | 0.67 |
| Rolofylline (KW-3902) | 0.66 |
| IWP-2 | 0.64 |
| T0901317 | 0.64 |
| Ritonavir | 0.63 |
| BIO | 0.62 |
| Amuvatinib | 0.61 |
| FRAX597 | 0.61 |
| Anti mullerian hormone | 0.59 |
| Wnt3a | 0.59 |
| Decernotinib | 0.58 |
| Dorsomorphin | 0.57 |
| Etomidate | 0.56 |
| GDC-0879 | 0.55 |

Table 12 provides the log 2 fold change for compounds that were observed to significantly increase expression of LDLR, encoding low density lipoprotein receptor, and/or decrease of expression of ANGPTL3 and/or PCSK9, encoding angiopoietin like 3 and proprotein convertase subtilisin/kexin type 9 respectively, which are associated with dyslipidemia.

TABLE 12
RNA-seq results for ANGPTL3, LDLR, and PCSK9

| | Fold Change (Log2) | | |
|---|---|---|---|
| Compounds | ANGPTL3 | LDLR | PCSK9 |
| WYE-125132 | −0.42 | 1.46 | −0.91 |
| (WYE-132) | | | |
| Pifithrin-u | −0.46 | 0.60 | −0.54 |
| LY294002 | 0 | 0.48 | −0.51 |
| SGI-1776 | −0.52 | 0.48 | 0 |
| Preladenant | −0.73 | 1.07 | 0 |
| CO-1686 | −0.67 | 0.51 | 0 |
| (Rociletinib) | | | |

Table 13 provides the log 2 fold change for additional compounds that were observed to significantly decrease expression of ANGPTL3, encoding angiopoietin like 3, which is associated with dyslipidemia.

TABLE 13
RNA-seq results for ANGPTL3

| | ANGPTL3-LINEAR |
|---|---|
| NAME | FOLD CHANGE |
| Fedratinib (SAR302503, TG101348) | 0.111878134 |
| Pentamidine isethionate | 0.132127255 |
| Romidepsin (FK228, Depsipeptide) | 0.13490353 |
| Pamidronate | 0.152830035 |
| AEE788 (NVP-AEE788) | 0.168404197 |
| JNJ-26854165 (Serdemetan) | 0.181746565 |
| GSK1059615 | 0.185565446 |
| LY2090314 | 0.189464571 |
| Carglumic Acid | 0.197510328 |
| MI-773 (SAR405838) | 0.20166044 |
| BI 2536 | 0.204475515 |
| Enzastaurin | 0.217637641 |
| AZD1080 | 0.223756268 |
| siRNA_FOS | 0.230046913 |
| Azacitidine | 0.231647015 |
| Cobimetinib (GDC-0973, RG7420) | 0.233258248 |
| Semaxinib | 0.244855074 |
| 10 uM | 0.248273124 |
| Simvastatin | 0.258816231 |
| Pilaralisib (XL147) | 0.26061644 |
| Tamoxifen | 0.26061644 |
| PH-797804 | 0.273573425 |
| MLN8054 | 0.275476279 |
| TFP | 0.283220971 |
| Amlodipine Besylate | 0.303548721 |
| Smoothened Agonist | 0.303548721 |
| Sunitinib | 0.303548721 |
| Isosorbide Dinitrate | 0.305660069 |
| Olmutinib (HM61713, | 0.305660069 |
| BI 1482694) | |
| Afatinib | 0.307786103 |
| GSK461364 | 0.307786103 |
| Belizatinib (TSR-011) | 0.312082637 |
| AZD2858 | 0.318640157 |
| FICZ | 0.323088208 |
| Fluvoxamine maleate | 0.323088208 |
| Risperidone | 0.323088208 |
| siRNA_TCF7L2_with Wnt3a | 0.334481889 |
| BGJ398 | 0.336808394 |
| Foretinib (GSK1363089) | 0.336808394 |
| Tanespimycin (17-AAG) | 0.336808394 |
| Linifanib (ABT-869) | 0.339151082 |
| Mubritinib (TAK 165) | 0.339151082 |
| BMP4 | 0.346277367 |
| YM155 (Sepantronium Bromide) | 0.348685917 |
| Vorinostat (SAHA, MK0683) | 0.351111219 |
| Proguanil | 0.353553391 |
| Lifirafenib (BGB-283) | 0.358488812 |
| SCIO-469 | 0.371130893 |
| AM 580 | 0.373712312 |
| siRNA_SREBF2_with SIMVASTATIN | 0.373712312 |
| NORCISAPRIDE HYDROCHLORIDE | 0.376311687 |
| Cinacalcet HCl | 0.378929142 |
| Sertraline HCl | 0.381564802 |
| TAK-285 | 0.384218795 |
| Succinobucol | 0.38958229 |
| ASP3026 | 0.392292049 |
| CD 2665 | 0.395020656 |
| RU 28318 | 0.400534939 |
| Piperacetazine | 0.40332088 |
| SIRNA_HNF1A | 0.411795509 |
| SIRNA_RBJ | 0.411795509 |
| Amodiaquine dihydrochloride dihydrate | 0.41754396 |
| Voxtalisib (XL765, SAR245409) | 0.41754396 |
| Tivozanib | 0.420448208 |
| LDN193189 | 0.420448208 |
| Cediranib | 0.432268616 |
| SKL2001 | 0.432268616 |
| SIRNA_NR1H3_WITH T0901317 | 0.432268616 |
| VX-702 | 0.435275282 |
| PF-3814735 | 0.438302861 |
| 1(3-Chlorophenyl)piperazine HCl | 0.441351498 |
| SIRNA_TEAD1 | 0.447512535 |
| GDC-0349 | 0.450625231 |
| Zafirlukast | 0.450625231 |
| PF-04691502 | 0.450625231 |
| Rimonabant | 0.453759578 |
| GSK2334470 | 0.453759578 |
| Ibutilide Fumarate | 0.453759578 |
| Chloroquine Phosphate | 0.453759578 |
| TAK-715 | 0.453759578 |
| Darapladib | 0.456915725 |
| Linifanib | 0.463294031 |
| MI-773 | 0.473028823 |
| Lansoprazole | 0.476318999 |
| Acitretin | 0.476318999 |
| Riluzole | 0.476318999 |
| Calcitriol | 0.482968164 |
| Everolimus (RAD001) | 0.482968164 |
| Sonidegib (Erismodegib, NVP-LDE225) | 0.489710149 |
| OSU-03012 | 0.496546248 |
| AZD8055 | 0.496546248 |
| Bumetanide | 0.5 |
| GW3965 | 2.070529848 |
| T0901317 | 2.848100391 |

Results herein provide evidence that the compounds shown in Tables 3-13 to have a significant therapeutic effect may be used to rescue the phenotype for the disease associated with the target gene. Additional genes within same pathway or controlled by the same signaling center as the target gene may also be modulated by compounds in Tables 3-13.

### Example 4: Upregulation of SERPINC1 in Hepatocytes

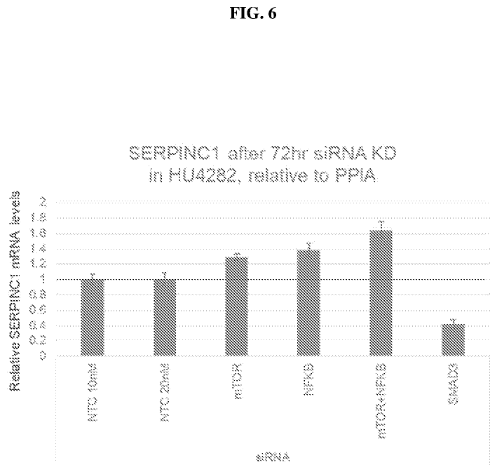

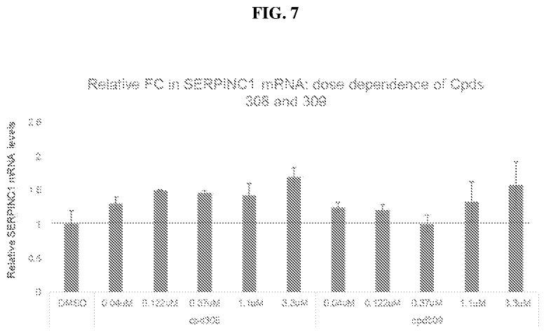

Compounds were tested in hepatocytes for upregulation of SERPINC1 mRNA for identification of potential treatments of SERPINC1/AT III deficiency. Tables 14-16 shows the quantitative PCR results of primary human hepatocytes and mouse hepatocytes treated with select compounds, and RNA seq results for MGH54 cells treated with select compounds. FIG. 6 shows an upregulation of SERPINC1 mRNA after 72 h treatment with siRNA targeted against mTOR and NFKB, relative to non-targeted control siRNA (NTC). FIG. 7 shows a dose dependent upregulation of SERPINC1 in response to treatment with compound 308 (OSI-027) and compound 309 (PF04691502) relative to DMSO control.

TABLE 14

| | | | | MGH54 |
|---|---|---|---|---|
| | | H8290 | | (RNA seq) |
| Name | MoA | 1 uM | 10 uM | 10 uM |
| OSI-027 | mTOR | 1.7 | 1.9 | NA |
| CZ415 | mTOR | 1.4 | 2.3 | NA |
| AZD-4055 | mTOR | 1.3 | 1.9 | NA |
| Temsirolimus | mTOR | 1.2 | 1.6 | 0 |
| Voxtalisib | mTOR | 1.2 | 1.5 | 0 |
| AZD8055 | mTOR | | | 0 |
| Lifirafenib | VEGFR/PDGFR | 1.1 | 2.1 | 1.43 |
| (BGB-283)/BeiGene | | | | |
| Foretinib | VEGFR/PDGFR | 1 | 2.2 | 0 |
| XL288 | VEGFR/PDGFR | 1 | 2 | NA |
| BMS-214662 | Farnesyl transferase | 0.9 | 2.4 | 2.46 |
| PF-3814735 | Aurora kinase | | | 2.43 |
| SB1317/TG02 | CDk2/JAK2/FLT3 | | | NA |
| AT9283 | JAK2/3; Aurora kinase | | | NA |

TABLE 15

| | | Lonza Hu4282 | |
|---|---|---|---|
| Name | MoA | 1 uM | 10 uM |
| OSI-027 | mTOR | | |
| CZ415 | mTOR | 1.25 | 1.42 |
| AZD-4055 | mTOR | 1.15 | 1.41 |
| Voxtalisib | mTOR | 1.1 | 1.24 |
| AZD8055 | mTOR | 1.3 | 1.61 |
| Lifirafenib (BGB-283)/BeiGene | VEGFR/PDGFR | 1.05 | 1.21 |
| Foretinib | VEGFR/PDGFR | 1.04 | 1.36 |
| XL288 | VEGFR/PDGFR | 1.19 | 2.45 |
| BMS-214662 | Farnesyl transferase | 0.98 | 1.23 |
| PF-3814735 | Aurora kinase | 0.99 | 2.6 |
| SB1317/TG02 | CDk2/JAK2/FLT3 | 1.9 | 2.28 |
| AT9283 | JAK2/3; Aurora kinase | 0.91 | 2.2 |

TABLE 16

| | | Mouse Heps | |
|---|---|---|---|
| Name | MoA | 1 uM | 10 uM |
| OSI-027 | mTOR | 1.53 | 2.36 |
| CZ415 | mTOR | 2.2 | 2.5 |
| AZD-4055 | mTOR | 2.4 | 2.7 |
| Voxtalisib | mTOR | 1.23 | 2.4 |
| AZD8055 | mTOR | 3.2 | 3.4 |
| Lifirafenib (BGB-283)/BeiGene | VEGFR/PDGFR | 0.81 | 1.1 |
| Foretinib | VEGFR/PDGFR | 1.05 | 0.93 |
| XL288 | VEGFR/PDGFR | 1.13 | 1.76 |
| BMS-214662 | Farnesyl transferase | 0.92 | 0.86 |
| PF-3814735 | Aurora kinase | 1.2 | 1.86 |
| SB1317/TG02 | CDk2/JAK2/FLT3 | 1.13 | 1.85 |
| AT9283 | JAK2/3; Aurora kinase | 1.11 | 1.81 |

### Example 5: Upregulation of MECP2 by Selected Compounds

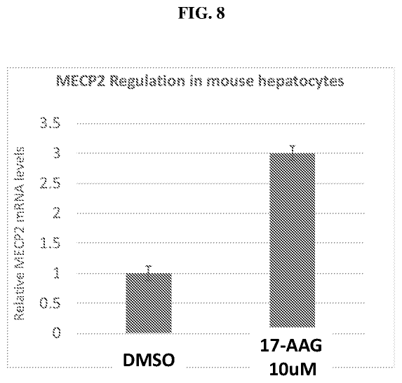

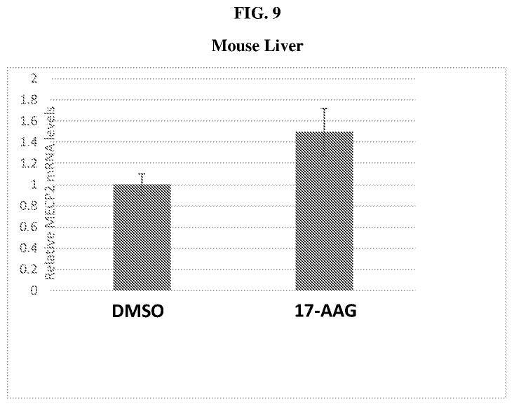

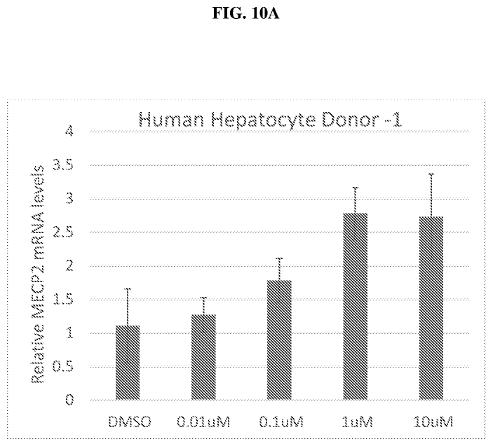

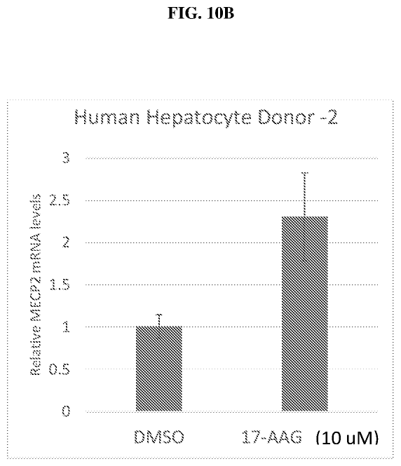

17-AAG was tested in hepatocytes for upregulation of MECP2 mRNA. Treament with 17-AAG resulted in increased MECP2 mRNA in mouse hepatocytes (FIG. 8) and mouse liver (FIG. 9) as detected by qPCR relative to DMSO control. Primary human hepatocytes from two donors exhibited a dose-dependent increase in MECP2 mRNA when treated with 17-AAG (FIGS. 10A and 10B). Additional compounds were tested for induction of MECP2 mRNA in human hepatocytes (Tables 17-19).

TABLE 17

| | | Stellate | |
|---|---|---|---|
| | | MECP2 (Avg_FC) | |
| Target | Compound name | 1 uM | 10 uM |
| mTORC1/2 | torin1 | 1.87 | 3.65 |
| Receptor tyrosine kinase, | Linifanib (ABT-869) | 2.50 | 3.17 |
| PDGFR, VEGFR | | | |
| PI3K(α/β/δ/γ)/mTOR | PF-04691502 | 1.27 | 2.78 |
| c-RET, VEGFR | Regorafenib (BAY 73- | 2.31 | 2.77 |
| | 4506) | | |
| JNK inhibitor | CC-401 hydrochloride | 1.92 | 2.69 |
| mTOR1/2 | INK-128 | 1.33 | 2.42 |
| PARP1 | Iniparib (BSI-201) | 2.39 | 2.26 |
| PI3Kα/δ/γ | Pilaralisib (XL147) | 2.28 | 2.21 |
| HSP90 | 17-AAG (Tanespimycin)/ | 2.43 | 2.21 |
| | KOS-953 | | |
| GSK-3 | LY2090314 | 2.65 | 2.20 |
| PKCβ1 and PKCβ2 | LY333531 HCl | 1.26 | 2.15 |
| Aurora Kinase A | MK-5108 (VX-689) | 2.17 | 2.01 |
| HSP90α/β | HSP-990 | 1.98 | 1.99 |
| MET | BMS 777607 | 0.97 | 1.92 |
| PPARg agonist | Pioglitazone HCl | 1.94 | 1.86 |

TABLE 18

| | | Human hep donor-1 | |
|---|---|---|---|
| | | MECP2 (Avg_FC) | |
| Target | Compound name | 1 uM | 10 uM |
| mTORC1/2 | torin1 | n/a | n/a |
| Receptor tyrosine kinase, | Linifanib (ABT-869) | 1.78 | 2.01 |
| PDGFR, VEGFR | | | |
| PI3K(α/β/δ/γ)/mTOR | PF-04691502 | n/a | n/a |
| c-RET, VEGFR | Regorafenib (BAY 73- | 2.05 | 1.92 |
| | 4506) | | |
| JNK inhibitor | CC-401 hydrochloride | 1.84 | 2.03 |
| mTOR1/2 | INK-128 | n/a | n/a |
| PARP1 | Iniparib (BSI-201) | 2.28 | 2.15 |
| PI3Kα/δ/γ | Pilaralisib (XL147) | 1.95 | 1.75 |
| HSP90 | 17-AAG (Tanespimycin)/ | 1.63 | 2.80 |
| | KOS-953 | | |
| GSK-3 | LY2090314 | 2.43 | 1.94 |
| PKCβ1 and PKCβ2 | LY333531 HCl | 1.44 | 2.14 |
| Aurora Kinase A | MK-5108 (VX-689) | 2.42 | 2.35 |
| HSP90α/β | HSP-990 | 2.54 | 2.72 |
| MET | BMS 777607 | n/a | n/a |
| PPARg agonist | Pioglitazone HCl | 2.21 | 2.27 |

TABLE 19

| | | Human hep donor-2 | |
|---|---|---|---|
| | | MECP2 (Avg_FC) | |
| Target | Compound name | 1 uM | 10 uM |
| mTORC1/2 | torin1 | 1.34 | 1.79 |
| Receptor tyrosine kinase, | Linifanib (ABT-869) | n/a | n/a |
| PDGFR, VEGFR | | | |
| PI3K(α/β/δ/γ)/mTOR | PF-04691502 | 1.13 | 1.88 |
| c-RET, VEGFR | Regorafenib (BAY 73- | n/a | n/a |
| | 4506) | | |
| JNK inhibitor | CC-401 hydrochloride | n/a | n/a |
| mTOR1/2 | INK-128 | 2.16 | 2.52 |
| PARP1 | Iniparib (BSI-201) | n/a | n/a |
| PI3Kα/δ/γ | Pilaralisib (XL147) | n/a | n/a |
| GSK-3 | LY2090314 | n/a | n/a |
| PKCβ1 and PKCβ2 | LY333531 HCl | n/a | n/a |
| Aurora Kinase A | MK-5108 (VX-689) | n/a | n/a |
| HSP90α/β | HSP-990 | 1.27 | 1.66 |
| MET | BMS 777607 | 1.35 | 3.04 |
| PPARg agonist | Pioglitazone HCl | n/a | n/a |

### Example 6: Downregulation of APOL Expression by Selected Compounds

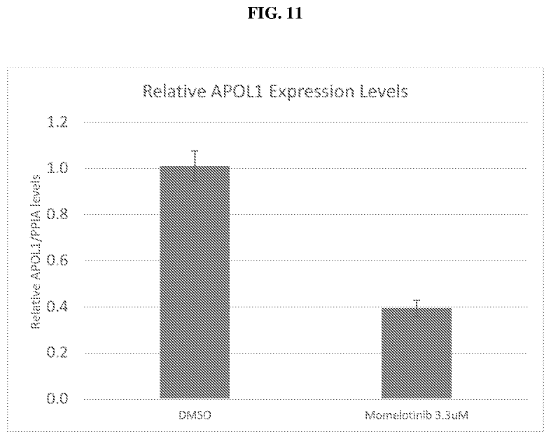

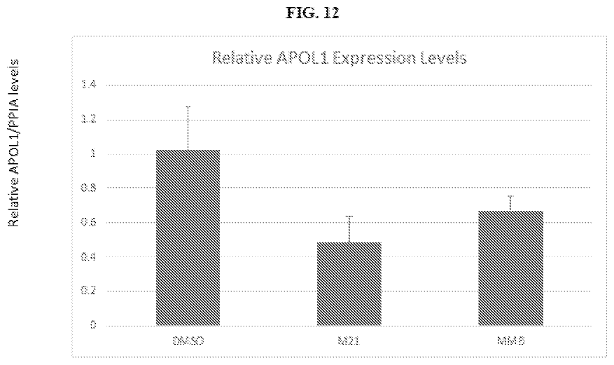

Downregulation of APOL mRNA was observed in primary human hepatocytes upon treatment with 3.3 uM Momelotenib (FIG. 11) or a Momelotenib metabolite, M21 (FIG. 12). Additional compounds were identified by RNAseq as described above that exhibited a downregulation of APOL mRNA at 10 uM dose (Table 20).

TABLE 20

| Compounds | linear fold change |
|---|---|
| CPD602_GSK1059615 | 0.51050606 |
| CPD309_PF-04691502 | 0.51405691 |
| CPD551_Sunitinib | 0.61985385 |
| CPD569_Bortezomib (PS-341) | 0.63728031 |
| CPD388_Methylergonovine | 0.64171295 |
| CPD513_Sertraline HCl | 0.64617642 |
| CPD377_Pyrvinium pamoate salt | 0.65067093 |
| CPD341_Cevimeline | 0.6551967 |
| CPD446_Mitotane | 0.6551967 |
| CPD447_Prednisolone | 0.6551967 |
| CPD15_Nitrofurantoin | 0.65975396 |

### Equivalents and Scope

Those skilled in the art will recognize, or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

1. A method of treating a subject with Fibronectin Glomerulopathy, comprising administering to the subject an effective amount of a compound from Table 3 capable of reducing the expression of a FN1 gene.

2. A method of reducing the expression of a FN1 gene in a cell, comprising introducing into the cell an effective amount of a compound from Table 3 capable of altering one or more signaling molecules associated with the regulatory sequence regions (RSRs) or portion thereof of the FN1 gene.

3. The method of claim 1 or claim 2, wherein the compound is selected from the group consisting of smoothened agonist, Crizotinib, BGJ398, AZD2858, and Amlodipine Besylate.

4. A method of treating a subject with Hereditary coproporphyria, comprising administering to the subject an effective amount of a compound from Table 4 capable of increasing the expression of a CPOX gene.

5. A method of increasing the expression of a CPOX gene in a cell, comprising introducing into the cell an effective amount of a compound from Table 4 capable of altering one or more signaling molecules associated with the regulatory sequence regions (RSRs) or portion thereof of the CPOX gene.

6. The method of claim 4 or claim 5, wherein the compound is selected from the group consisting of 17-AAG, Cobalt chloride, SKL2001, FICZ, and prednisone.

7. A method of treating a subject with SERPINC1 Deficiency, comprising administering to the subject an effective amount of a compound from Table 5 capable of increasing the expression of a SERPINC1 gene.

8. A method of increasing the expression of a SERPINC1 gene in a cell, comprising introducing into the cell an effective amount of a compound from Table 5, Table 14, Table 15 or Table 16 capable of altering one or more signaling molecules associated with the regulatory sequence regions (RSRs) or portion thereof of the SERPINC1 gene.

9. The method of claim 7 or claim 8, wherein the compound is selected from the group consisting of OSI-027, PF04691502, CP-673451, Echinomycin, and Pacritinib (SB1518).

10. A method of treating a subject with Alagille Syndrome, comprising administering to the subject an effective amount of a compound from Table 6 capable of increasing the expression of a JAG1 gene and/or a NOTCH2 gene.

11. A method of increasing the expression of a JAG1 gene and/or a NOTCH2 gene in a cell, comprising introducing into the cell an effective amount of a compound from Table 6 capable of altering one or more signaling molecules associated with the regulatory sequence regions (RSRs) or portion thereof of the JAG1 gene and/or the NOTCH2 gene.

12. The method of claim 10 or claim 11, wherein the compound is selected from the group consisting of Merestinib and Torcetrapib.

13. A method of treating a subject with Glycogen Storage disease 1b, comprising administering to the subject an effective amount of a compound from Table 7 capable of increasing the expression of a SLC37A4 gene.

14. A method of increasing the expression of a SLC37A4 gene in a cell, comprising introducing into the cell an effective amount of a compound from Table 7 capable of altering one or more signaling molecules associated with the regulatory sequence regions (RSRs) or portion thereof of the SLC37A4 gene.

15. The method of claim 13 or claim 14, wherein the compound is selected from the group consisting of Echinomycin, prednisone, CP-673451, and cobalt chloride.

16. A method of treating a subject with Acute Intermittent porphyria, comprising administering to the subject an effective amount of a compound from Table 8 capable of increasing the expression of a HMBS gene.

17. A method of increasing the expression of a HMBS gene in a cell, comprising introducing into the cell an effective amount of a compound from Table 8 capable of altering one or more signaling molecules associated with the regulatory sequence regions (RSRs) or portion thereof of the HMBS gene.

18. The method of claim 16 or claim 17, wherein the compound is sotrastaurin.

19. A method of treating a subject with LECT2 amyloidosis, comprising administering to the subject an effective amount of a compound from Table 9 capable of reducing the expression of a LECT2 gene.

20. A method of reducing the expression of a LECT2 gene in a cell, comprising introducing into the cell an effective amount of a compound from Table 9 capable of altering one or more signaling molecules associated with the regulatory sequence regions (RSRs) or portion thereof of the LECT2 gene.

21. The method of claim 19 or claim 20, wherein the compound is selected from the group consisting of calcitrol, 17-AAG and Ritaonavir.

22. A method of treating a subject with APOL1-associated glomerular disease, comprising administering to the subject an effective amount of a compound from Table 10 or Table 16 capable of reducing the expression of a APOL1 gene.

23. A method of reducing the expression of a APOL1 gene in a cell, comprising introducing into the cell an effective amount of a compound from Table 10 or Table 16 capable of altering one or more signaling molecules associated with the regulatory sequence regions (RSRs) or portion thereof of the APOL1 gene.

24. The method of claim 22 or claim 23, wherein the compound is selected from the group consisting of Nitrofurantoin, Crizotinib, Momelotenib, and Momelotenib metabolite M21.

25. A method of treating a subject with Gilbert Syndrome or Criggler Najjar, type II, comprising administering to the subject an effective amount of a compound from Table 11 capable of increasing the expression of a UGT1A1 gene.

26. A method of increasing the expression of a UGT1A1 gene in a cell, comprising introducing into the cell an effective amount of a compound from Table 11 capable of altering one or more signaling molecules associated with the regulatory sequence regions (RSRs) or portion thereof of the UGT1A1 gene.

27. The method of claim 25 or claim 26, wherein the compound is selected from the group consisting of FICZ, Kartogenin, meBIO, CP-673451, BAM7, and EW-7197.

28. A method of treating a subject with dyslipidemia, comprising administering to the subject an effective amount of a compound from Table 12 capable of increasing the expression of a LDLR gene, and/or reducing the expression of a ANGPTL3 gene and/or PCSK9 gene.

29. A method of modulating the expression of at least one gene selected from the group consisting of ANGPTL3, LDLR, and PCSK9 genes in a cell, comprising introducing into the cell an effective amount of a compound from Table 12 or Table 13 capable of altering one or more signaling molecules associated with the regulatory sequence regions (RSRs) or portion thereof of the ANGPTL3, LDLR, or PCSK9 genes.

30. The method of claim 28 or claim 29, wherein the compound is selected from the group consisting of WYE-125132, Pifithrin-u, LY294002, SGI-1776, Preladenant, and CO-1686.

31. A method of treating a subject with Rett Syndrome, comprising administering to the subject an effective amount of a compound from Table 17, Table 18 or Table 19 capable of increasing the expression of a MECP2 gene.

32. A method of treating a subject with Rett Syndrome, comprising administering to the subject an effective amount of a compound from Table 17, Table 18 or Table 19 capable of increasing the expression of a MECP2 gene.

33. The method of claim 31 or 32, wherein the compound is 17-AAG.

34. The method of any one of the above claims, wherein the subject is human.
